(19) United States
(12) Patent Application Publication
(10) Pub. No.: US 2007/0010532 A1
(43) Pub. Date: Jan. 11, 2007

(54) 3-Pyridylcarboxamide derivatives as pesticidal agents

(75) Inventors: Koichi Araki, Ibaraki (JP); Tetsuya Murata, Tochigi (JP); Masahito Ito, Ibaraki (JP); Norihiko Nakakura, Kawachi-gun (JP); Eiichi Shimojo, Tochigi (JP); Christian Arnold, Langenfeld (DE); Daniela Jans, Homburg (DE); Waltraud Hempel, Liederbach (DE); Olga Malsam, Rosrath (DE)

Correspondence Address: STERNE, KESSLER, GOLDSTEIN & FOX PLLC, 1100 NEW YORK AVENUE, N.W., WASHINGTON, DC, 20005, US

(73) Assignee: BAYER CROPSCIENCE GMBH, Frankfurt (DE)

(21) Appl. No.: 10/562,919

(22) PCT Filed: Jun. 18, 2004
(86) PCT No.: PCT/EP04/06610

§ 371 Date: Jun. 12, 2006

(30) Foreign Application Priority Data
Jul. 1, 2003 (EP) 03014913.2

(51) Int. Cl. A01N 43/54; A01N 43/40; C07D 417/02; C07D 403/02

(52) U.S. Cl. 514/256; 514/340; 544/333; 546/269.7; 546/272.7

(57) ABSTRACT

The invention relates to a 3- pyridylcarboxamide derivative of formula (I): wherein the various symbols are as defined in the description, to compositions thereof, to their use for the control of pests, and to processes for their preparation.

The invention relates to 3-pyridylcarboxamide derivatives and their use for the control of pests, in particular arthropods such as insects and acarids, and helminths (including nematodes); to compositions containing them, and to processes and intermediates for their preparation.

The control of insects with 3-pyridylcarboxamide compounds has been described in many patent applications such as EP 580374, JP 10101648, JP 10182625, WO 200109104, WO 200114340, JP 6321903, JP 10195072 and JP 11180957.

However, the level of action and/or duration of action of these prior-art compounds is not entirely satisfactory in all fields of application, in particular against certain organisms or when low concentrations are applied.

Since modern pesticides must meet a wide range of demands, for example regarding level, duration and spectrum of action, use spectrum, toxicity, combination with other active substances, combination with formulation auxiliaries or synthesis, and since the occurrence of resistances is possible, the development of such substances can never be regarded as concluded, and there is constantly a high demand for novel compounds which are advantageous over the known compounds, at least as far as some aspects are concerned.

The present invention provides a compound which is a 3-pyridylcarboxamide derivative of formula (I): 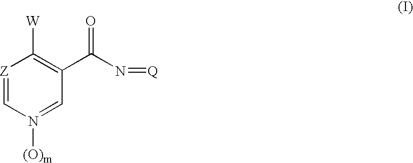
wherein:
W is (C₁-C₄)haloalkyl;
Z is CH or N;
=Q is a group of formula (A) or (B): 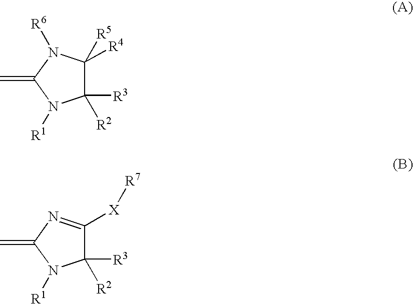
R¹ and R⁶ are each independently H, (C₁-C₈)alkyl, (C₃-C₆)alkenyl, (C₃-C₆)alkynyl, (C₁-C₆)alkoxy, (C₃-C₆)alkenyloxy, (C₃-C₆)alkynyloxy, (C₁-C₆)alkylamino, di-(C₁-C₆)alkylamino, NHCO(C₁-C₆)alkyl, NHSO₂(C₁-C₆)alkyl, CO(C₁-C₆)alkyl or SO₂(C₁-C₆)alkyl which last twelve mentioned groups are unsubstituted or substituted by one or more R⁸ groups; or are (C₃-C₈)cycloalkyl or (C₃-C₈)cycloalkyl-(C₁-C₆)alkyl- which cycloalkyl radicals are unsubstituted or substituted by one or more (C₁-C₆)alkyl, (C₁-C₆)haloalkyl or R⁸ groups; or are —(CR⁹R¹⁰)ₚR¹¹, —(CR⁹R¹⁰)ₚheterocyclyl, OH, SO₂R¹¹, NH₂, NHCOR¹¹, NH(C₃-C₈)cycloalkyl, NH(CR⁹R¹⁰)ₛR¹¹, O(CR⁹R¹⁰)ᵣR¹¹, —(CR⁹R¹⁰)CO₂CH₂R¹¹, O(CH₂)ᵣheterocyclyl, N═C[(C₁-C₆)alkyl]₂, COR^{11a} or CO-heterocyclyl; or are (C₃-C₆)alkenyl substituted by R^{11a};
R², R³, R⁴ and R⁵ are each independently H, (C₁-C₈)alkyl, (C₂-C₆)alkenyl or (C₂-C₆)alkynyl, which last three mentioned groups are unsubstituted or substituted by one or more R⁸ groups; or are (C₃-C₈)cycloalkyl or (C₃-C₈)cycloalkyl-(C₁-C₆)alkyl- which cycloalkyl radicals are unsubstituted or substituted by one or more (C₁-C₆)alkyl, (C₁-C₆)haloalkyl or R⁸ groups; or are (C₁-C₆)alkyl-SH, —(CR⁹R¹⁰)ₚR¹¹, —(CR⁹R¹⁰)ₚheterocyclyl or O(CH₂)ᵣR¹¹;
or R² and R³, or R⁴ and R⁵ together with the respective attached carbon atom form a carbonyl or thiocarbonyl group or a (C₃-C₈)cycloalkyl ring; or an imino group which is unsubstituted or substituted by (C₁-C₆)alkyl, CO(C₁-C₆)alkyl or R^{11a};
R⁷ is (C₃-C₆)alkenyl, (C₃-C₆)alkynyl, —(CR⁹R¹⁰)ₚR¹¹, —(CR⁹R¹⁰)ₚheterocyclyl, CO(C₁-C₆)alkyl or a (C₃-C₈)cycloalkyl ring; or (C₁-C₈)alkyl unsubstituted or substituted by one or more radicals selected from halogen and —OC(═O)—(C₁-C₄)alkyl;
R⁸ is halogen, (C₁-C₆)alkoxy, (C₁-C₆)haloalkoxy, S(O)ₙR¹², CN, CO₂(C₁-C₆)alkyl, CO₂H, NO₂, OH, amino, (C₁-C₆)alkylamino, di-(C₁-C₆)alkylamino, carbamoyl, (C₁-C₆)-alkylcarbamoyl, di-(C₁-C₆)-alkylcarbamoyl, CH[O(C₁-C₆)alkyl]₂, (C₃-C₆)alkenyloxy, (C₃-C₆)alkynyloxy or O(CH₂)ᵣR¹¹;
R⁹ and R¹⁰ are each independently H, (C₁-C₆)alkyl or (C₁-C₆)haloalkyl;
R¹¹ is aryl unsubstituted or substituted by one or more radicals selected from the group consisting of (C₁-C₆)alkyl, (C₁-C₆)haloalkyl, (C₂-C₆)alkenyl, (C₂-C₆)alkynyl, (C₃-C₈)cycloalkyl, —(CH₂)ᵤR^{11a}, heterocyclyl, halogen, (C₁-C₆)alkoxy, (C₁-C₆)haloalkoxy, S(O)ₙR¹², CN, CO₂(C₁-C₆)alkyl, NO₂, amino, (C₁-C₆)alkylamino, di-(C₁-C₆)alkylamino and CO(C₁-C₆)alkyl;
R^{11a} is aryl unsubstituted or substituted by one or more radicals selected from the group consisting of (C₁-C₆)alkyl, (C₁-C₆)haloalkyl, halogen, (C₁-C₆)alkoxy, (C₁-C₆)haloalkoxy, S(O)ₙR¹², CN, CO₂(C₁-C₆)alkyl, CO₂H, NO₂, OH, amino, (C₁-C₆)alkylamino and di-(C₁-C₆)alkylamino;
R¹² is (C₁-C₆)alkyl or (C₁-C₆)haloalkyl;
X is O, S, NR¹³ or NOR¹³;
R¹³ is H, (C₁-C₈)alkyl, (C₃-C₆)alkenyl, (C₃-C₆)alkynyl or (C₃-C₈)cycloalkyl which last four mentioned groups are unsubstituted or substituted by one or more R⁸ groups; or is (C₃-C₈)cycloalkyl-(C₁-C₆)alkyl- which cycloalkyl is unsubstituted or substituted by one or more (C₁-C₆)alkyl, (C₁-C₆)haloalkyl or R⁸ groups; or is —(CR⁹R¹⁰)ₚR¹¹ or —(CR⁹R¹⁰)ₚheterocyclyl;
m, s and u are each independently 0 or 1;
n is 0, 1 or 2;
p is 0, 1, 2 or 3;
r is 0 or an integer from 1 to 6; and each heterocyclyl in the above mentioned radicals is independently a heterocyclic radical having 3 to 7 ring atoms and 1 to 4 hetero atoms selected from N, O and S, and is unsubstituted or substituted by one or more radicals selected from the group consisting of (C₁-C₆)alkyl, (C₁-C₆)haloalkyl, (C₂-C₆)alkenyl, (C₂-C₆)alkynyl, —(CH₂)ᵤR^{11a}, halogen, (C₁-C₆)alkoxy, (C₁-C₆)haloalkoxy, S(O)ₙR¹², CN, CO₂(C₁-C₆)alkyl, NO₂, OH, amino, (C₁-C₆)alkylamino and di-(C₁-C₆)alkylamino; or a pesticidally acceptable salt thereof.

These compounds possess valuable pesticidal properties.

The invention also encompasses any stereoisomer, enantiomer or geometric isomer, and mixtures thereof.

By the term “pesticidally acceptable salts” is meant salts the cations or anions of which are known and accepted in the art for the formation of salts for pesticidal or horticultural use. Suitable salts with bases, e.g. formed by compounds of formula (I) containing a carboxy, NH or OH group, include alkali metal (e.g. sodium and potassium), alkaline earth metal (e.g. calcium and magnesium), ammonium and amine (e.g. diethanolamine, triethanolamine, octylamine, morpholine and dioctylmethylamine) salts. Suitable acid addition salts, e.g. formed by compounds of formula (I) containing an amino group, include salts with inorganic acids, for example hydrochlorides, sulphates, phosphates and nitrates and salts with organic acids for example acetic acid.

The term pests means arthropod pests (including insects and acarids), and helminths (including nematodes).

In the present patent application, including the accompanying claims, the aforementioned substituents have the following meanings:
halogen atom means fluorine, chlorine, bromine or iodine;
alkyl groups and portions thereof (unless otherwise defined) may be straight- or branched-chain;
cycloalkyl groups preferably have from three to six carbon atoms in the ring and are optionally substituted by halogen or alkyl.

The haloalkyl and haloalkoxy groups bear one or more halogen atoms; preferred groups of this type include —CF₃ and —OCF₃.

The term “halo” before the name of a radical means that this radical is partially or completely halogenated, that is to say, substituted by F, Cl, Br, or I, in any combination, preferably by F or Cl.

The expression, “(C₁-C₆)-alkyl” is to be understood as meaning an unbranched or branched hydrocarbon radical having 1, 2, 3, 4, 5 or 6 carbon atoms, such as, for example a methyl, ethyl, propyl, isopropyl, 1-butyl, 2-butyl, 2-methylpropyl or tert-butyl radical.

“(C₁-C₆)-Haloalkyl” is to be understood as meaning an alkyl group mentioned under the expression “(C₁-C₆)-alkyl” in which one or more hydrogen atoms are replaced by the same number of identical or different halogen atoms, preferably by chlorine or fluorine, such as the trifluoromethyl, the 1-fluoroethyl, the 2,2,2-trifluoroethyl, the chloromethyl, fluoromethyl, the difluoromethyl or the 1,1,2,2-tetrafluoroethyl group.

“(C₁-C₆)-Alkoxy” is to be understood as meaning an alkoxy group whose hydrocarbon radical has the meaning given under the expression “(C₁-C₆)-alkyl”.

The terms “alkenyl” and “alkynyl” with a range of carbon atoms stated as prefix denote a straight-chain or branched hydrocarbon radical having a number of carbon atoms which corresponds to this stated range and which contains at least one multiple bond which can be located in any position of the respective unsaturated radical. “(C₂-C₆)-Alkenyl” accordingly denotes, for example, the vinyl, alkyl, 2-methyl-2-propenyl, 2-butenyl, pentenyl, 2-methylpentenyl or the hexenyl group. “(C₂-C₆)-Alkynyl” denotes, for example, the ethynyl, propargyl, 2-methyl-2-propynyl; 2-butynyl; 2-pentynyl or the 2-hexynyl group.

“(C₃-C₈)-Cycloalkyl” denotes monocyclic alkyl radicals, such as the cyclopropyl, cyclobutyl, cyclopentyl, cyclohexyl, cycloheptyl or cyclooctyl radical, and denotes bicyclic alkyl radicals, such as the norbornyl radical.

The expression “(C₃-C₈)-cycloalkyl-(C₁-C₆)-alkyl” is to be understood as meaning, for example the cyclopropylmethyl, cyclopentylmethyl, cyclohexylmethyl, cyclohexylethyl, cyclohexylbutyl, 1-methylcyclopropyl, 1-methylcyclopentyl, 1-methylcyclohexyl, 3-hexylcyclobutyl or the 4-tert-butylcyclohexyl radical.

“(C₁-C₆)-Alkylamino” denotes a nitrogen atom which is substituted by an alkyl radical of the above definition. “Di-(C₁-C₆)-alkylamino” denotes a nitrogen atom which is substituted by two alkyl radical of the above definition.

The expression “(C₁-C₆)-alkylcarbamoyl” denotes a carbamoyl group having one hydrocarbon radical which has the meaning given under the expression “(C₁-C₆)-alkyl”; and “di-(C₁-C₆)-alkylcarbamoyl” denotes a carbamoyl group having two hydrocarbon radicals which can be identical or different.

The expression “aryl” is to be understood as meaning a carbocyclic, i.e. constructed of carbon atoms, aromatic radical having preferably 6 to 14, in particular 6 to 12, carbon atoms, such as, for example, phenyl, naphthyl or biphenylyl, preferably phenyl.

The expression “heterocyclyl” denotes a saturated, partially saturated or aromatic ring system having 3 to 7 ring atoms and 1 to 4 heteroatoms selected from the group consisting of O, S and N, where at least one carbon atom has to be present in the ring.

Preferably “heterocyclyl” denotes a thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, 1,3-benzodioxole, 1,3-benzodioxane, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, morpholine, piperidine, piperazine, pyrroline, pyrrolidine, oxazoline, tetrahydrofuran, tetrahydropyran, isoxazolidine, oxazolidine, thiazoline, thiazolidine, oxirane or oxetane radical.

More preferably, “heterocyclyl” denotes a pyridine, pyrimidine, 1,2,4-oxadiazole, 1,3,4-oxadiazole, pyrrole, furan, thiophene, oxazole, thiazole, imidazole, pyrazole, isoxazole, 1,2,4-triazole, 1,2,3,4-tetrazole, pyrazine, pyridazine, oxazoline, thiazoline, tetrahydrofuran, tetrahydropyran, morpholine, piperidine, piperazine, pyrroline, pyrrolidine, oxazolidine, thiazolidine, oxirane, oxetane, 1,3-benzodioxole or 1,3-benzodioxane radical.

The “heterocyclyl” radical is unsubstituted or substituted by one or more radicals selected from the group consisting of (C₁-C₆)alkyl, (C₁-C₆)haloalkyl, (C₂-C₆)alkenyl, (C₂-C₆)alkynyl, —(CH₂)ᵤR^{11a}, halogen, (C₁-C₆)alkoxy, (C₁-C₆)haloalkoxy, S(O)ₙR¹², CN, CO₂(C₁-C₆)alkyl, NO₂, OH, amino, (C₁-C₆)alkylamino and di-(C₁-C₆)alkylamino.

Preferably the “heterocyclyl” radical is unsubstituted or substituted by one or more radicals selected from the group consisting of halogen, CN, NO₂, (C₁-C₄)-alkyl, (C₁-C₄)-haloalkyl, (C₁-C₄)-alkoxy, (C₁-C₄)-haloalkoxy, (C₁-C₄)-alkylthio and (C₁-C₄)-haloalkylthio.

It is to be generally understood, unless otherwise stated, that the term “unsubstituted or substituted by one or more groups” or “unsubstituted or substituted by one or more groups selected from” means that such groups (or preferred groups) may be the same or different.

Preferably W is CF₃.

Preferably Z is CH.

Preferably R¹ and R⁶ are each independently H, (C₁-C₈)alkyl, (C₃-C₆)alkenyl, CO(C₁-C₆)alkyl or SO₂(C₁-C₆)alkyl; or are —(CR⁹R¹⁰)ₚR¹¹ (more preferably R¹ is (C₁-C₈)alkyl, (C₃-C₆)alkenyl or —(CR⁹R¹⁰)ₚR¹¹ and R⁶ is H or (C₁-C₈)alkyl).

Preferably R², R³, R⁴ and R⁵ are each independently H, (C₁-C₈)alkyl, (C₃-C₆)alkenyl, (C₃-C₆)alkynyl, —(CR⁹R¹⁰)ₚR¹¹, —(CR⁹R¹⁰)ₚheterocyclyl or O(CH₂)ᵣR¹¹; or R² and R³ together with the attached carbon atom form a carbonyl or thiocarbonyl group, or an imino group which is unsubstituted or substituted by (C₁-C₆)alkyl, CO(C₁-C₆)alkyl or R^{11a}; or R² and R³, or R⁴ and R⁵ together with the respective attached carbon atom form a (C₃-C₈)cycloalkyl ring.

Preferably R⁷ is (C₁-C₈)alkyl, (C₃-C₆)alkenyl, (C₃-C₆)alkynyl, —(CR⁹R¹⁰)ₚR¹¹ or —(CR⁹R¹⁰)ₚheterocyclyl.

Preferably R⁸ is halogen, (C₁-C₄)alkoxy or OH (more preferably R⁸ is halogen).

Preferably R⁹ and R¹⁰ are each independently H, (C₁-C₄)alkyl or (C₁-C₄)haloalkyl.

Preferably R¹¹ is phenyl unsubstituted or substituted by one or more radicals selected from the group consisting of (C₁-C₄)alkyl, (C₁-C₄)haloalkyl, (C₂-C₄)alkenyl, (C₂-C₄)alkynyl, (C₃-C₆)cycloalkyl, —(CH₂)ᵤR^{11a}, heterocyclyl, halogen, (C₁-C₄)alkoxy, (C₁-C₄)haloalkoxy, S(O)ₙR¹², CN, CO₂(C₁-C₄)alkyl, NO₂, amino, (C₁-C₄)alkylamino and di-(C₁-C₄)alkylamino; (more preferably R¹¹ is phenyl unsubstituted or substituted by one or more radicals selected from the group consisting of (C₁-C₄)alkyl, halogen, (C₁-C₄)alkoxy, NO₂ and amino).

Preferably R^{11a} is phenyl unsubstituted or substituted by one or more radicals selected from the group consisting of (C₁-C₄)alkyl, (C₁-C₄)haloalkyl, halogen, (C₁-C₄)alkoxy, (C₁-C₄)haloalkoxy, S(O)ₙR¹², CN, CO₂(C₁-C₄)alkyl, CO₂H, NO₂, OH, amino, (C₁-C₄)alkylamino and di-(C₁-C₄)alkylamino.

Preferably R¹² is (C₁-C₄)alkyl or (C₁-C₄)haloalkyl.

Preferably X is O or S.

Preferably m is 0.

Preferably p, r, s and u are each independently 0 or 1.

Preferably each heterocyclyl in the above mentioned radicals is independently a heterocyclic radical having 3 to 7 ring atoms and 1 to 4 hetero atoms selected from N, O and S.

A preferred class of compounds are those wherein:
W is CF₃;
Z is CH;
R¹ and R⁶ are each independently H, (C₁-C₈)alkyl, (C₃-C₆)alkenyl, CO(C₁-C₆)alkyl or SO₂(C₁-C₆)alkyl; or are —(CR⁹R¹⁰)ₚR¹¹ (more preferably R¹ is (C₁-C₈)alkyl, (C₃-C₆)alkenyl or —(CR⁹R¹⁰)ₚR¹¹ and R⁶ is H or (C₁-C₈)alkyl);
R², R³, R⁴ and R⁵ are each independently H, (C₁-C₈)alkyl, (C₃-C₆)alkenyl, (C₃-C₆)alkynyl, —(CR⁹R¹⁰)ₚR¹¹, —(CR⁹R¹⁰)ₚheterocyclyl or O(CH₂)ᵣR¹¹; or R² and R³ together with the attached carbon atom form a carbonyl or thiocarbonyl group, or an imino group which is unsubstituted or substituted by (C₁-C₆)alkyl, CO(C₁-C₆)alkyl or R^{11a}; or R² and R³, or R⁴ and R⁵ together with the respective attached carbon atom form a (C₃-C₈)cycloalkyl ring;
R⁷ is (C₁-C₈)alkyl, (C₃-C₆)alkenyl, (C₃-C₆)alkynyl, —(CR⁹R¹⁰)ₚR¹¹ or —(CR⁹R¹⁰)ₚheterocyclyl;
R⁸ is (C₁-C₄)alkoxy or OH;
R⁹ and R¹⁰ are each independently H, (C₁-C₄)alkyl or (C₁-C₄)haloalkyl;
R¹¹ is phenyl unsubstituted or substituted by one or more radicals selected from the group consisting of (C₁-C₄)alkyl, (C₁-C₄)haloalkyl, (C₂-C₄)alkenyl, (C₂-C₄)alkynyl, (C₃-C₆)cycloalkyl, —(CH₂)ᵤR^{11a}, heterocyclyl, halogen, (C₁-C₄)alkoxy, (C₁-C₄)haloalkoxy, S(O)ₙR¹², CN, CO₂(C₁-C₄)alkyl, NO₂, amino, (C₁-C₄)alkylamino and di-(C₁-C₄)alkylamino; (more preferably R¹¹ is phenyl unsubstituted or substituted by one or more radicals selected from the group consisting of (C₁-C₄)alkyl, halogen, (C₁-C₄)alkoxy, NO₂ and amino);
R^{11a} is phenyl unsubstituted or substituted by one or more radicals selected from the group consisting of (C₁-C₄)alkyl, (C₁-C₄)haloalkyl, halogen, (C₁-C₄)alkoxy, (C₁-C₄)haloalkoxy, S(O)ₙR¹², CN, CO₂(C₁-C₄)alkyl, CO₂H, NO₂, OH, amino, (C₁-C₄)alkylamino and di-(C₁-C₄)alkylamino;
R¹² is (C₁-C₄)alkyl or (C₁-C₄)haloalkyl;
X is O or S;
m is 0;
p, r, s and u are each independently 0 or 1; and
each heterocyclyl in the above mentioned radicals is independently a heterocyclic radical having 3 to 7 ring atoms and 1 to 4 hetero atoms selected from N, O and S.

A particularly preferred embodiment of the invention comprises compounds of formula (I) wherein:
W is CF₃;
Z is CH;
=Q is a group of formula (A1): 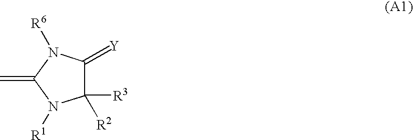
R¹ and R⁶ are each independently H, (C₁-C₈)alkyl, (C₃-C₆)alkenyl, CO(C₁-C₆)alkyl or SO₂(C₁-C₆)alkyl; or are —(CR⁹R¹⁰)ₚR¹¹ (more preferably R¹ is (C₁-C₈)alkyl, (C₃-C₆)alkenyl or —(CR⁹R¹⁰)ₚR¹¹, and R⁶ is H or (C₁-C₈)alkyl);
R² and R³ are each independently H, (C₁-C₈)alkyl, (C₃-C₆)alkenyl, (C₃-C₆)alkynyl, —(CR⁹R¹⁰)ₚR¹¹, —(CR⁹R¹⁰)ₚheterocyclyl or O(CH₂)ᵣR¹¹;
Y is O or S; and
heterocyclyl is a heterocyclic radical having 3 to 7 ring atoms and 1 to 4 hetero atoms selected from N, O and S.

A further preferred class of compounds are those wherein:
W is CF₃;
Z is CH;
=Q is a group of formula (A);
R¹ is H, (C₁-C₄)alkyl, (C₃-C₄)alkenyl or —(CH₂)ₚphenyl;
R² is H or —(CH₂)ₚphenyl; or (C₁-C₄)alkyl unsubstituted or substituted by OH, carbamoyl, S(O)ₙR¹² or SH;
R³ is H or (C₁-C₄)alkyl;
or R² and R³ together with the attached carbon atom form a (C₃-C₆)cycloalkyl ring;
R⁴ and R⁵ together with the attached carbon atom form a thiocarbonyl group;
R⁶ is H;
R¹² is (C₁-C₄)alkyl;
p is 0 or 1; and
m is 0.

A further preferred class of compounds are those wherein:
W is CF₃;
Z is CH;
=Q is a group of formula (A);
R¹ is H, (C₁-C₄)alkyl, (C₃-C₄)alkenyl or —(CH₂)ₚphenyl;
R² is H or —(CH₂)ₚphenyl; or (C₁-C₄)alkyl unsubstituted or substituted by OH, carbamoyl, S(O)ₙR¹² or SH;
R³ is H or (C₁-C₄)alkyl;
or R² and R³ together with the attached carbon atom form a (C₃-C₆)cycloalkyl ring;
R⁴ and R⁵ together with the attached carbon atom form a carbonyl group;
R⁶ is H or (C₃-C₄)alkenyl; or (C₁-C₄)alkyl unsubstituted or substituted by CO₂(C₁-C₄)alkyl or CO₂CH₂phenyl; or —CH₂R¹¹ in which R¹¹ is phenyl unsubstituted or substituted by one or more halogen groups; or phenyl unsubstituted or substituted by one or more radicals selected from (C₁-C₄)alkyl and (C₁-C₄)haloalkyl;
R¹² is (C₁-C₄)alkyl;
p is 0 or 1; and
m is 0.

A further preferred class of compounds are those wherein:
W is CF₃;
Z is CH;
=Q is a group of formula (A);
R¹ is H, (C₁-C₄)alkyl, (C₃-C₄)alkenyl or —(CH₂)ₚphenyl;
R² is H or —(CH₂)ₚphenyl; or (C₁-C₄)alkyl unsubstituted or substituted by OH, carbamoyl, S(O)ₙR¹² or SH;
R³ is H or (C₁-C₄)alkyl;
or R² and R³ together with the attached carbon atom form a (C₃-C₆)cycloalkyl ring;
R⁴ and R⁵ together with the attached carbon atom form an imino group;
R⁶ is H or (C₃-C₄)alkenyl; or (C₁-C₄)alkyl unsubstituted or substituted by CO₂(C₁-C₄)alkyl or CO₂CH₂phenyl; or —CH₂R¹¹ in which R¹¹ is phenyl unsubstituted or substituted by one or more halogen atoms; or phenyl unsubstituted or substituted by one or more radicals selected from (C₁-C₄)alkyl and (C₁-C₄)haloalkyl;
R¹² is (C₁-C₄)alkyl;
p is 0 or 1; and
m is 0.

A further preferred class of compounds are those wherein:
W is CF₃;
Z is CH;
=Q is a group of formula (A);
R¹ is H, (C₁-C₄)alkyl, (C₃-C₄)alkenyl or —CH₂phenyl; or phenyl unsubstituted or substituted by one or more radicals selected from (C₁-C₄)alkyl and (C₁-C₄)haloalkyl; or —CH₂heterocyclyl wherein heterocyclyl is a heterocyclic radical having 5 or 6 ring atoms and 1 or 2 hetero atoms selected from N and S, and is unsubstituted or substituted by one or more radicals selected from the group consisting of (C₁-C₆)alkyl, (C₁-C₆)haloalkyl and halogen;
R², R³, R⁴ and R⁵ are each H;
R⁶ is H, (C₁-C₄)alkyl, (C₃-C₄)alkenyl or —(CH₂)ₚphenyl;
p is 0 or 1; and
m is 0.

A further preferred class of compounds are those wherein:
W is CF₃;
Z is CH;
=Q is a group of formula (B);
R¹ is H, (C₁-C₄)alkyl, (C₃-C₄)alkenyl, —(CH₂)ₚphenyl, CO(C₁-C₄)alkyl or COphenyl;
R² is H or —(CH₂)ₚphenyl; or (C₁-C₄)alkyl unsubstituted or substituted by OH, carbamoyl, S(O)ₙR¹² or SH;
R³ is H or (C₁-C₄)alkyl;
or R² and R³ together with the attached carbon atom form a (C₃-C₆)cycloalkyl ring;
X is S;
R⁷ is (C₃-C₄)alkenyl or —CH₂phenyl; or (C₁-C₄)alkyl unsubstituted or substituted by —OC(═O)—(C₁-C₄)alkyl;
R¹² is (C₁-C₄)alkyl;
p is 0 or 1; and
m is 0.

A further preferred class of compounds are those wherein:
W is CF₃;
Z is CH;
=Q is a group of formula (B);
R¹ is H, (C₁-C₄)alkyl, (C₃-C₄)alkenyl, —(CH₂)ₚphenyl, CO(C₁-C₄)alkyl or COphenyl;
R² is H or —(CH₂)ₚphenyl; or (C₁-C₄)alkyl unsubstituted or substituted by OH, carbamoyl, S(O)ₙR¹² or SH;
R³ is H or (C₁-C₄)alkyl;
or R² and R³ together with the attached carbon atom form a (C₃-C₆)cycloalkyl ring;
X is O;
R⁷ is (C₃-C₄)alkenyl or —CH₂phenyl; or (C₁-C₄)alkyl unsubstituted or substituted by —OC(═O)—(C₁-C₄)alkyl;
R¹² is (C₁-C₄)alkyl;
p is 0 or 1; and
m is 0.

The compounds of general formula (I) can be prepared by the application or adaptation of known methods (i.e. methods heretofore used or described in the chemical literature).

In the following description of processes when symbols appearing in formulae are not specifically defined, it is understood that they are “as defined above” in accordance with the first definition of each symbol in the specification.

According to a feature of the invention compounds of formula (I) wherein W and Z are as defined above, =Q is a group of formula (A), R² and R³ are as defined above excluding where together with the attached carbon atom they form a carbonyl, thiocarbonyl or imino group, R⁴ and R⁵ together with the attached carbon atom form a thiocarbonyl group, R¹ and R⁶ are each a hydrogen atom and m is zero, may be prepared by the cyclisation-rearrangement reaction of a compound of formula (II): 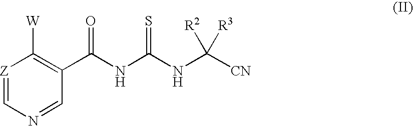
wherein W and Z are as defined above, R² and R³ are as defined above excluding where together with the attached carbon atom they form a carbonyl, thiocarbonyl or imino group. The reaction is generally performed in a solvent such as dioxan, tetrahydrofuran or N,N-dimethylformamide, at a temperature of from 0° to 100° C. (preferably 0° to 50° C.), and preferably in the presence of an organic base such as a tertiary amine for example triethylamine, or pyridine, or an inorganic base such as an alkali metal carbonate, for example potassium carbonate, or an alkali metal alkoxide such as sodium ethoxide, or sodium hydride. When a base is not present the temperature range is preferably 50° C. to 100° C. The reaction proceeds via cyclisation to an intermediate of formula (III), which is generally not isolated: 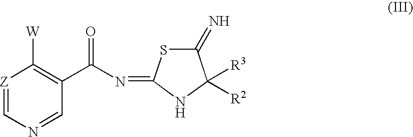
wherein W, Z, R² and R³ are as defined above, which rearranges to the compound of formula (I).

According to a further feature of the invention compounds of formula (I) wherein W and Z are as defined above, =Q is a group of formula (A), R² and R³ are as defined above excluding where they form a carbonyl, thiocarbonyl or imino group, R⁴ and R⁵ together with the attached carbon atom form a thiocarbonyl group, R¹ and R⁶ are each a hydrogen atom and m is zero, may also be prepared by the reaction of a compound of formula (IV): 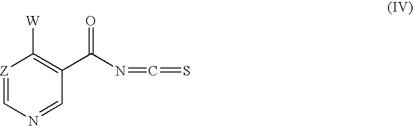
wherein W and Z are as defined above, with a compound of formula (V):
H₂NCR²(R³)CN (V)
wherein R² and R³ are as defined above excluding where together with the attached carbon atom they form a carbonyl, thiocarbonyl or imino group, to give the corresponding compound of formula (II), which then undergoes the above described cyclisation-rearrangement reaction.

The reaction is generally performed in the presence of an organic base such as a tertiary amine for example triethylamine, or pyridine, or an inorganic base such as an alkali metal carbonate, for example potassium carbonate, or an alkali metal alkoxide such as sodium ethoxide, or sodium hydride, in a solvent such as dioxan, tetrahydrofuran or N,N-dimethylformamide, at a temperature of from 0° to 100° C. (preferably 0° to 50° C.).

According to a further feature of the invention compounds of formula (I) wherein =Q is a group of formula (A), R¹ is a hydrogen atom, R² and R³ are as defined above excluding where together with the attached carbon atom they form a carbonyl, thiocarbonyl or imino group, R⁴ and R⁵ together with the attached carbon atom form a carbonyl group, W, Z and R⁶ are as defined above and m is zero, may be prepared by the reaction of a compound of formula (VI): 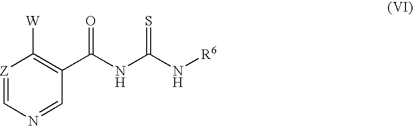
wherein W, Z and R⁶ are as defined above, with a compound of formula (VII):
H₂NCR²(R³)CO₂R⁷ (VIII)
wherein R² and R³ are as defined above excluding where together with the attached carbon atom they form a carbonyl, thiocarbonyl or imino group, and R⁷ is a leaving group, generally alkyl such as methyl or ethyl, or benzyl.

The reaction is generally performed in the presence of a coupling agent such as a carbodiimide for example N,N-dicyclohexylcarbodiimide, or 1-ethyl-3-(3-dimethylaminopropyl)carbodiimide or an acid salt thereof such as the hydrochloride salt, in a solvent such as dioxan, tetrahydrofuran or N,N-dimethylformamide, at a temperature of from 200 to 120° C., and optionally in the presence of a catalyst such as 4-dimethylaminopyridine.

The reaction proceeds via an intermediate compound of formula (VIII) which may be isolated if required: 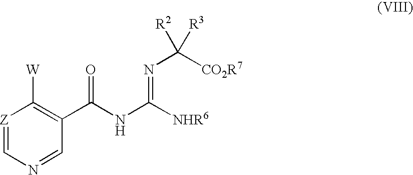
wherein the various symbols are as defined above, followed by cyclisation.

According to a further feature of the invention compounds of formula (I) wherein =Q is a group of formula (A) or (B), m is zero and the other symbols are as defined above, may be prepared by the acylation of the corresponding compound of formula (A¹) or (B¹): 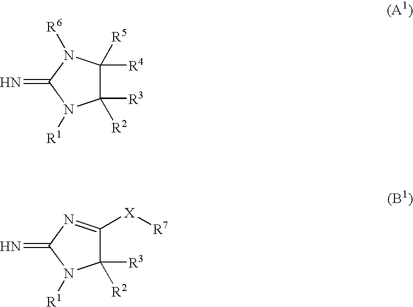
wherein the various symbols are as defined above, with a compound of formula (IX): 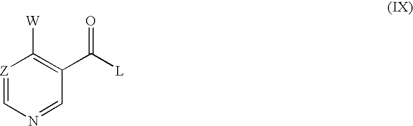
wherein W and Z are as defined above, L is a leaving group, generally halogen and preferably chlorine. The reaction is generally performed in a solvent such as dichloromethane, at a temperature of from 0° to 100° C. (preferably 0° to 50° C.).

According to a further feature of the invention compounds of formula (I) wherein =Q is a group of formula (B), W, Z, R¹ and R⁷ are as defined above, X is S, m is zero, and R² and R³ are as defined above excluding where together with the attached carbon atom they form a carbonyl or thiocarbonyl group, or an imino group which is unsubstituted or substituted by (C₁-C₆)alkyl, CO(C₁-C₆)alkyl or R^{11a}, may be prepared by the reaction of a compound of formula (I) which is of formula (X): 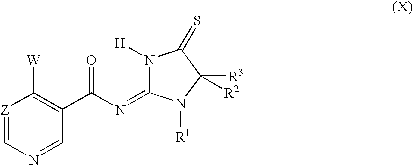
wherein W, Z, R¹, R² and R³ are as defined above, with a compound of formula (XI):
R⁷L (XI)
wherein R⁷ is as defined above and L is a leaving group generally halogen and preferably chlorine or bromine. The reaction is generally performed in the presence of an organic base such as a tertiary amine for example triethylamine, or pyridine, or an inorganic base such as an alkali metal carbonate, for example potassium carbonate, or an alkali metal alkoxide such as sodium ethoxide, or sodium hydride, in a solvent such as dioxan, tetrahydrofuran or N,N-dimethylformamide, at a temperature of from 0° to 100° C. (preferably 0° to 50° C.).

According to a further feature of the invention compounds of formula (I) wherein =Q is a group of formula (A), W, Z, R¹, R², R³, R⁴ and R⁵ are as defined above, R⁶ is hydrogen and m is zero, may be prepared by cyclising a compound of formula (XII): 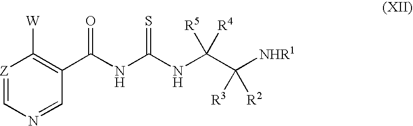
wherein W, Z, R¹, R², R³, R⁴ and R⁵ are as defined above.

The reaction is generally performed in the presence of an organic base such as a tertiary amine for example triethylamine, or pyridine, or an inorganic base such as an alkali metal carbonate, for example potassium carbonate, or an alkali metal alkoxide such as sodium ethoxide, or sodium hydride, and a carbodiimide for example N,N-dicyclohexylcarbodiimide or 1-ethyl-3-(3-dimethylaminopropyl)carbodiimide, in a solvent such as dioxan, tetrahydrofuran or N,N-dimethylformamide, at a temperature of from 0° to 100° C. (preferably 0° to 50° C.).

According to a further feature of the invention compounds of formula (I) wherein =Q is a group of formula (A), W, Z, R¹, R² and R³ are as defined above, R⁴ and R⁵ together with the attached carbon atom form a carbonyl group, R⁶ is hydrogen, and m is zero, may be prepared by the oxidation and hydrolysis reaction of a compound of formula (I) wherein Q is a group of formula (B), X is S, and W, Z, R¹, R², R³ and R⁷ are as defined above, and m is zero. The reaction is generally performed in the presence of an oxidising agent such as hydrogen peroxide in a solvent such as acetic acid, or a peracid such as 3-chloroperbenzoic acid in a solvent such as dichloromethane or 1,2-dichloroethane, at a temperature of from 0° C. to the reflux temperature of the solvent.

According to a further feature of the invention compounds of formula (I) wherein =Q is a group of formula (B), W, Z, R², R³ and R⁷ are as defined above, R¹ is CO(C₁-C₆)alkyl which is unsubstituted or substituted by one or more R^{a} groups, or is COR^{11a} or CO-heterocyclyl, and m is zero, may be prepared by the acylation of the corresponding compound of formula (I) wherein R¹ is hydrogen. The reaction is generally performed using an acylating agent of formula (XIII):
R¹COL (XIII)
wherein L is a leaving group generally halogen, preferably chlorine, in a solvent such as dichloromethane or 1,2-dichloroethane, at a temperature of from 0° C. to the reflux temperature of the solvent.

According to a further feature of the invention compounds of formula (I) wherein =Q is a group of formula (A), W, Z, R², R³, R⁴, R⁵ and R⁶ are as defined above, R¹ is CO(C₁-C₆)alkyl which is unsubstituted or substituted by one or more R⁸ groups, or is COR^{11a} or CO-heterocyclyl, and m is zero, may be prepared by the acylation of the corresponding compound of formula (I) wherein R¹ is hydrogen. The reaction is generally performed using an acylating agent of formula (XIII) as defined above, in a solvent such as dichloromethane or 1,2-dichloroethane, at a temperature of from 0° C. to the reflux temperature of the solvent.

According to a further feature of the invention compounds of formula (I) wherein Q is as defined above, and m is 1 may be prepared by oxidising a corresponding compound in which m is 0. The oxidation is generally performed using hydrogen peroxide in a solvent such as acetic acid, or a peracid such as 3-chloroperbenzoic acid in a solvent such as dichloromethane or 1,2-dichloroethane, at a temperature of from 0° C. to the reflux temperature of the solvent.

Intermediates of formula (II), (VI) or (XII) may be prepared by the reaction of a compound of formula (IV) with a corresponding compound of formula (V), (XIV) or (XV) respectively: 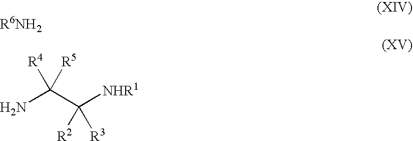
wherein R¹, R², R³, R⁴, R⁵ and R⁶ are as defined above. The reaction is generally performed in an inert solvent such as dichloroethane or tetrahydrofuran at a temperature of from 0° to 60° C.

Intermediates of formula (IX) wherein L is chlorine, may be prepared according to known procedures, for example by the reaction of the corresponding carboxylic acid wherein L is replaced by OH, with a suitable halogenating agent, preferably oxalyl chloride, in a solvent such as dichloroethane, optionally in the presence of N,N-dimethylformamide, at a temperature of from 0° to 60° C.

Intermediates of formula (IV) may be prepared according to known procedures, for example by the reaction of a compound of formula (IX) as defined above, with an alkali metal thiocyanate or ammonium thiocyanate or tetraalkylammonium thiocyanate for example tetrabutylammonium thiocyanate, in the presence of a base such as an alkali metal carbonate for example potassium carbonate, at a temperature of from 0° to 60° C.

Collections of compounds of the formula (I) which can be synthesized by the above mentioned process may also be prepared in a parallel manner, and this may be effected manually or in a semiautomated or fully automated manner. In this case, it is possible, for example, to automate the procedure of the reaction, work-up or purification of the products or of the intermediates. In total, this is to be understood as meaning a procedure as is described, for example, by S. H. DeWitt in “Annual Reports in Combinatorial Chemistry and Molecular Diversity: Automated Synthesis”, Volume 1, Verlag Escom 1997, pages 69 to 77.

A series of commercially available apparatuses as are offered by, for example, Stem Corporation, Woodrolfe Road, Tollesbury, Essex, CM9 8SE, England or H+P Labortechnik GmbH, Bruckmannring 28, 85764 Oberschleiβheim, Germany or Radleys, Shirehill, Saffron Walden, Essex, England, may be used for the parallel procedure of the reaction and work-up. For the parallel purification of compounds of the formula (I), or of intermediates obtained during the preparation, use may be made, inter alia, of chromatography apparatuses, for example those by ISCO, Inc., 4700 Superior Street, Lincoln, Nebr. 68504, USA.

The apparatuses mentioned lead to a modular procedure in which the individual process steps are automated, but manual operations must be performed between the process steps. This can be prevented by employing semi-integrated or fully integrated automation systems where the automation modules in question are operated by, for example, robots. Such automation systems can be obtained, for example, from Zymark Corporation, Zymark Center, Hopkinton, Mass. 01748, USA.

In addition to what has been described here, compounds of the formula (I) may be prepared in part or fully by solid-phase-supported methods. For this purpose, individual intermediate steps or all intermediate steps of the synthesis or of a synthesis adapted to suit the procedure in question are bound to a synthetic resin. Solid-phase-supported synthesis methods are described exdensively in the specialist literature, for example Barry A. Bunin in “The Combinatorial Index”, Academic Press, 1998.

The use of solid-phase-supported synthesis methods permits a series of protocols which are known from the literature and which, in turn, can be performed manually or in an automated manner. For example, the “tea-bag method” (Houghten, U.S. Pat. No. 4,631,211; Houghten et al., Proc. Natl. Acad. Sci, 1985, 82, 5131-5135), in which products by IRORI, 11149 North Torrey Pines Road, La Jolla, Calif. 92037, USA, are employed, may be semiautomated. The automation of solid-phase-supported parallel syntheses is performed successfully, for example, by apparatuses by Argonaut Technologies, Inc., 887 Industrial Road, San Carlos, Calif. 94070, USA or MultiSynTech GmbH, Wullener Feld 4, 58454 Witten, Germany.

The preparation of the processes described herein yields compounds of the formula (I) in the form of substance collections which are termed libraries. The present invention also provides libraries which comprise at least two compounds of the formula (I).

Compounds of formula (V), (VII), (XI), (XIII), (XIV), (XV), (A¹) and (B¹) are known or may be prepared by known methods.

Certain compounds of formula (XII) are novel and as such form a further feature of the invention.

The following non-limiting Examples illustrate the preparation of the compounds of formula (I).

### CHEMICAL EXAMPLES

NMR spectra were run in deuterochloroform unless stated otherwise.

In the Examples which follow, quantities (also percentages) are weight-based, unless stated otherwise.

### Example 1

1-(1-Cyano-1-methyl)ethyl-3-(4-trifluoromethyl-3-pyridylcarbonyl)thiourea (0.05 g) in methanol was heated at reflux for 1 hour. Ethyl acetate and water were added and the organic phase dried (magnesium sulfate) and evaporated to give N-[(2Z)-4,4-dimethyl-5-thioxo-2-imidazolinylidene]-4-(trifluoromethyl)nicotinamide (0.05 g, Compound A-86).

### Example 2

Oxalyl chloride (6.4 ml, 2M) was added to a suspension of 4-trifluoromethylnicotinic acid (2 g) and a catalytic amount of N,N-dimethylformamide in dichloromethane, and stirred at 20° C. for 1 hour. After evaporation, the residue was dissolved in acetone and potassium thiocyanate (2 g) added with ice bath cooling to give 4-trifluoromethyl-3-pyridylcarbonyl isothiocyanate, then 2-amino-2-methylpropanenitrile (1.35 g) and potassium carbonate (1 g) were added and the mixture heated at reflux for 1 hour.

Ethyl acetate was added and the mixture washed with water, dried (magnesium sulfate), evaporated and the residue purified by silica-gel chromatography, eluting with n-hexane/ethyl acetate (3/1), to give N-[(2Z)-4,4-dimethyl-5-thioxo-2-imidazolinylidene]-4-(trifluoromethyl)nicotinamide (1.4 g, Compound A-86).

### Example 3

Sodium hydride (0.09 g, 60% dispersion in mineral oil) was added to a solution of N-[(2Z)-4,4-dimethyl-5-thioxo-2-imidazolinylidene]-4-(trifluoromethyl)nicotinamide (0.50 g) in tetrahydrofuran at 20° C., and stirred for 0.5 hour. Iodomethane (0.20 ml) was added to the mixture and heated at reflux for 1 hour. Ethyl acetate and water were added, the organic phase dried (magnesium sulfate) and evaporated to give N-[(2Z)-5,5-dimethyl-4-methylthio-1,5-dihydro-2-H-imidazol-2-ylidene]-4-(trifluoromethyl)nicotinamide (0.50 g, Compound E-129).

### Example 4

Acetyl chloride was added to a mixture of N-[(2Z)-5,5-dimethyl-4-methylthio-1,5-dihydro-2-H-imidazol-2-ylidene]-4-(trifluoromethyl)nicotinamide (0.20 g) and triethylamine (0.13 ml) in dichloromethane, and stirred for 1 hour. Ethyl acetate and water were added, the organic phase dried (magnesium sulfate) and evaporated to give N-[(2Z)-1-acetyl-5,5-dimethyl-4-methylthio-1,5-dihydro-2H-imidazol-2-ylidene]-4-(trifluoromethyl)nicotinamide (0.19 g, Compound E-135).

### Example 5

3-Chloroperoxybenzoic acid (0.1 g) was added to a solution of N-[(2Z)-5-ethyl-5-methyl-4-methylthio-1,5-dihydro-2H-imidazol-2-ylidene]-4-(trifluoro methyl)nicotinamide (0.1 g) in dichloromethane, and stirred at 20° C. for 1 hour.

The mixture was washed in turn with water, hydrochloric acid 1(M), saturated sodium bicarbonate and brine, dried (magnesium sulfate), evaporated and the residue purified by silica-gel chromatography, eluting with n-hexane/ethyl acetate (3/1), to give N-[(2Z)-4-ethyl-4-methyl-5-oxo-2-imidazolidinylidene]-4-(trifluoromethyl)nicotinamide (0.03 g, Compound B-91).

### Example 6

A mixture of 1-(4-chlorobenzyl)-3-(4-trifluoromethyl-3-pyridylcarbonyl)thiourea (0.40 g), 1-ethyl-3-(3-dimethylaminopropyl)carbodiimide hydrochloride (0.25 g), benzyl 2-methylalaninate (0.25 g) and a catalytic amount of 4-dimethylaminopyridine in tetrahydrofuran was heated at reflux for 1 hour. Ethyl acetate and water were added, the organic phase dried (magnesium sulfate), evaporated and the residue purified by silica-gel chromatography, eluting with n-hexane/ethyl acetate (5/1) to give N-[(2Z)-1-(4-chlorobenzyl)-4,4-dimethyl-5-oxo-2-imidazolidinylidene]-4-(trifluoromethyl)nicotinamide (0.36 g, Compound B-529).

Preparation of Intermediate Used in the Above Examples

Oxalyl chloride (8 ml, 2M) was added to a suspension 4-trifluoromethylnicotinic acid (5 g) and a catalytic amount of N,N-dimethylformamide in dichloromethane, and stirred at 20° C. for 1 hour. After evaporation, the residue was dissolved in acetone and potassium thiocyanate (5 g) added with ice bath cooling to give 4-trifluoromethyl-3-pyridylcarbonyl isothiocyanate. A solution of methylamine in Methaol (40%, 5 ml) was then added and the mixture heated at reflux for 1 hour. Ethyl acetate was added and the mixture washed with water, dried (magnesium sulfate), evaporated and the residue recrystallized (ethanol) to give 1-methyl-3-(4-trifluoromethyl-3-pyridylcarbonyl)thiourea (3.2 g); NMR 3.29(3H, d), 7.67(1H, d), 8.89(1H, s), 8.97 (1H, d), 10.30(1H, brs).

By proceeding in a similar manner the following intermediate was also prepared: 1-(1-cyano-1-methyl)ethyl-3-(4-trifluoromethyl-3-pyridylcarbonyl)thiourea, NMR 1.95(6H, s), 7.80(1H, d), 8.93(1H, s), 9.00 (1H, d), 10.46(1H, brs).

The following preferred compounds shown in Tables 1 to 6 also form part of the present invention, and were or may be prepared in accordance with, or analogously to, the above-mentioned Examples 1 to 6 or the above-described general methods. In the Tables Ph means phenyl and Me means methyl.

Compound numbers are given for reference purposes only.

TABLE 1
Compounds of formula (Ia):

| | |
|---|---|
| | (Ia) |
| 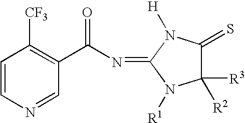 | |

| Compound | R¹ | R² | R³ |
|---|---|---|---|
| A-1 | H | H | H |
| A-2 | CH₃ | H | H |
| A-3 | CH₂CH═CH₂ | H | H |
| A-4 | CH₂Ph | H | H |
| A-5 | Ph | H | H |
| A-6 | H | CH₃ | H |
| A-7 | CH₃ | CH₃ | H |
| A-8 | CH₂CH═CH₂ | CH₃ | H |
| A-9 | CH₂Ph | CH₃ | H |
| A-10 | Ph | CH₃ | H |
| A-11 | H | CH₃CH₂ | H |
| A-12 | CH₃ | CH₃CH₂ | H |
| A-13 | CH₂CH═CH₂ | CH₃CH₂ | H |
| A-14 | CH₂Ph | CH₃CH₂ | H |
| A-15 | Ph | CH₃CH₂ | H |
| A-16 | H | n-C₃H₇ | H |
| A-17 | CH₃ | n-C₃H₇ | H |
| A-18 | CH₂CH═CH₂ | n-C₃H₇ | H |
| A-19 | CH₂Ph | n-C₃H₇ | H |
| A-20 | Ph | n-C₃H₇ | H |
| A-21 | H | i-C₃H₇ | H |
| A-22 | CH₃ | i-C₃H₇ | H |
| A-23 | CH₂CH═CH₂ | i-C₃H₇ | H |
| A-24 | CH₂Ph | i-C₃H₇ | H |
| A-25 | Ph | i-C₃H₇ | H |
| A-26 | H | n-C₄H₉ | H |
| A-27 | CH₃ | n-C₄H₉ | H |
| A-28 | CH₂CH═CH₂ | n-C₄H₉ | H |
| A-29 | CH₂Ph | n-C₄H₉ | H |
| A-30 | Ph | n-C₄H₉ | H |
| A-31 | H | i-C₄H₉ | H |
| A-32 | CH₃ | i-C₄H₉ | H |
| A-33 | CH₂CH═CH₂ | i-C₄H₉ | H |
| A-34 | CH₂Ph | i-C₄H₉ | H |
| A-35 | Ph | i-C₄H₉ | H |
| A-36 | H | s-C₄H₉ | H |
| A-37 | CH₃ | s-C₄H₉ | H |
| A-38 | CH₂CH═CH₂ | s-C₄H₉ | H |
| A-39 | CH₂Ph | s-C₄H₉ | H |
| A-40 | Ph | s-C₄H₉ | H |
| A-41 | H | t-C₄H₉ | H |
| A-42 | CH₃ | t-C₄H₉ | H |
| A-43 | CH₂CH═CH₂ | t-C₄H₉ | H |
| A-44 | CH₂Ph | t-C₄H₉ | H |
| A-45 | Ph | t-C₄H₉ | H |
| A-46 | H | CH₂Ph | H |
| A-47 | CH₃ | CH₂Ph | H |
| A-48 | CH₂CH═CH₂ | CH₂Ph | H |
| A-49 | CH₂Ph | CH₂Ph | H |
| A-50 | Ph | CH₂Ph | H |
| A-51 | H | Ph | H |
| A-52 | CH₃ | Ph | H |
| A-53 | CH₂CH═CH₂ | Ph | H |
| A-54 | CH₂Ph | Ph | H |
| A-55 | Ph | Ph | H |
| A-56 | H | CH₂OH | H |
| A-57 | CH₃ | CH₂OH | H |
| A-58 | CH₂CH═CH₂ | CH₂OH | H |
| A-59 | CH₂Ph | CH₂OH | H |
| A-60 | Ph | CH₂OH | H |
| A-61 | H | CH(OH)CH₃ | H |
| A-62 | CH₃ | CH(OH)CH₃ | H |
| A-63 | CH₂CH═CH₂ | CH(OH)CH₃ | H |
| A-64 | CH₂Ph | CH(OH)CH₃ | H |
| A-65 | Ph | CH(OH)CH₃ | H |
| A-66 | H | CH₂SH | H |
| A-67 | CH₃ | CH₂SH | H |
| A-68 | CH₂CH═CH₂ | CH₂SH | H |
| A-69 | CH₂Ph | CH₂SH | H |
| A-70 | Ph | CH₂SH | H |
| A-71 | H | CH₂SCH₂CH₃ | H |
| A-72 | CH₃ | CH₂SCH₂CH₃ | H |
| A-73 | CH₂CH═CH₂ | CH₂SCH₂CH₃ | H |
| A-74 | CH₂Ph | CH₂SCH₂CH₃ | H |
| A-75 | Ph | CH₂SCH₂CH₃ | H |
| A-76 | H | CH₂CONH₂ | H |
| A-77 | CH₃ | CH₂CONH₂ | H |
| A-78 | CH₂CH═CH₂ | CH₂CONH₂ | H |
| A-79 | CH₂Ph | CH₂CONH₂ | H |
| A-80 | Ph | CH₂CONH₂ | H |
| A-81 | H | CH₂CH₂CONH₂ | H |
| A-82 | CH₃ | CH₂CH₂CONH₂ | H |
| A-83 | CH₂CH═CH₂ | CH₂CH₂CONH₂ | H |
| A-84 | CH₂Ph | CH₂CH₂CONH₂ | H |
| A-85 | Ph | CH₂CH₂CONH₂ | H |
| A-86 | H | CH₃ | CH₃ |
| A-87 | CH₃ | CH₃ | CH₃ |
| A-88 | CH₂CH═CH₂ | CH₃ | CH₃ |
| A-89 | CH₂Ph | CH₃ | CH₃ |
| A-90 | Ph | CH₃ | CH₃ |
| A-91 | H | CH₃CH₂ | CH₃ |
| A-92 | CH₃ | CH₃CH₂ | CH₃ |
| A-93 | CH₂CH═CH₂ | CH₃CH₂ | CH₃ |
| A-94 | CH₂Ph | CH₃CH₂ | CH₃ |
| A-95 | Ph | CH₃CH₂ | CH₃ |
| A-96 | H | n-C₃H₇ | CH₃ |
| A-97 | CH₃ | n-C₃H₇ | CH₃ |
| A-98 | CH₂CH═CH₂ | n-C₃H₇ | CH₃ |
| A-99 | CH₂Ph | n-C₃H₇ | CH₃ |
| A-100 | Ph | n-C₃H₇ | CH₃ |
| A-101 | H | i-C₃H₇ | CH₃ |
| A-102 | CH₃ | i-C₃H₇ | CH₃ |
| A-103 | CH₂CH═CH₂ | i-C₃H₇ | CH₃ |
| A-104 | CH₂Ph | i-C₃H₇ | CH₃ |
| A-105 | Ph | i-C₃H₇ | CH₃ |
| A-106 | H | n-C₄H₉ | CH₃ |
| A-107 | CH₃ | n-C₄H₉ | CH₃ |
| A-108 | CH₂CH═CH₂ | n-C₄H₉ | CH₃ |
| A-109 | CH₂Ph | n-C₄H₉ | CH₃ |
| A-110 | Ph | n-C₄H₉ | CH₃ |
| A-111 | H | i-C₄H₉ | CH₃ |
| A-112 | CH₃ | i-C₄H₉ | CH₃ |
| A-113 | CH₂CH═CH₂ | i-C₄H₉ | CH₃ |
| A-114 | CH₂Ph | i-C₄H₉ | CH₃ |
| A-115 | Ph | i-C₄H₉ | CH₃ |
| A-116 | H | s-C₄H₉ | CH₃ |
| A-117 | CH₃ | s-C₄H₉ | CH₃ |
| A-118 | CH₂CH═CH₂ | s-C₄H₉ | CH₃ |
| A-119 | CH₂Ph | s-C₄H₉ | CH₃ |
| A-120 | Ph | s-C₄H₉ | CH₃ |
| A-121 | H | t-C₄H₉ | CH₃ |
| A-122 | CH₃ | t-C₄H₉ | CH₃ |
| A-123 | CH₂CH═CH₂ | t-C₄H₉ | CH₃ |
| A-124 | CH₂Ph | t-C₄H₉ | CH₃ |
| A-125 | Ph | t-C₄H₉ | CH₃ |
| A-126 | H | CH₂Ph | CH₃ |
| A-127 | CH₃ | CH₂Ph | CH₃ |
| A-128 | CH₂CH═CH₂ | CH₂Ph | CH₃ |
| A-129 | CH₂Ph | CH₂Ph | CH₃ |
| A-130 | Ph | CH₂Ph | CH₃ |
| A-131 | H | Ph | CH₃ |
| A-132 | CH₃ | Ph | CH₃ |
| A-133 | CH₂CH═CH₂ | Ph | CH₃ |
| A-134 | CH₂Ph | Ph | CH₃ |
| A-135 | Ph | Ph | CH₃ |
| A-136 | H | C₂H₅ | C₂H₅ |
| A-137 | CH₃ | C₂H₅ | C₂H₅ |
| A-138 | CH₂CH═CH₂ | C₂H₅ | C₂H₅ |
| A-139 | CH₂Ph | C₂H₅ | C₂H₅ |
| A-140 | Ph | C₂H₅ | C₂H₅ |
| A-141 | H | n-C₃H₇ | C₂H₅ |
| A-142 | CH₃ | n-C₃H₇ | C₂H₅ |
| A-143 | CH₂CH═CH₂ | n-C₃H₇ | C₂H₅ |
| A-144 | CH₂Ph | n-C₃H₇ | C₂H₅ |
| A-145 | Ph | n-C₃H₇ | C₂H₅ |
| A-146 | H | i-C₃H₇ | C₂H₅ |
| A-147 | CH₃ | i-C₃H₇ | C₂H₅ |
| A-148 | CH₂CH═CH₂ | i-C₃H₇ | C₂H₅ |
| A-149 | CH₂Ph | i-C₃H₇ | C₂H₅ |
| A-150 | Ph | i-C₃H₇ | C₂H₅ |
| A-151 | H | n-C₄H₉ | C₂H₅ |
| A-152 | CH₃ | n-C₄H₉ | C₂H₅ |
| A-153 | CH₂CH═CH₂ | n-C₄H₉ | C₂H₅ |
| A-154 | CH₂Ph | n-C₄H₉ | C₂H₅ |
| A-155 | Ph | n-C₄H₉ | C₂H₅ |
| A-156 | H | i-C₄H₉ | C₂H₅ |
| A-157 | CH₃ | i-C₄H₉ | C₂H₅ |
| A-158 | CH₂CH═CH₂ | i-C₄H₉ | C₂H₅ |
| A-159 | CH₂Ph | i-C₄H₉ | C₂H₅ |
| A-160 | Ph | i-C₄H₉ | C₂H₅ |
| A-161 | H | s-C₄H₉ | C₂H₅ |
| A-162 | CH₃ | s-C₄H₉ | C₂H₅ |
| A-163 | CH₂CH═CH₂ | s-C₄H₉ | C₂H₅ |
| A-164 | CH₂Ph | s-C₄H₉ | C₂H₅ |
| A-165 | Ph | s-C₄H₉ | C₂H₅ |
| A-166 | H | t-C₄H₉ | C₂H₅ |
| A-167 | CH₃ | t-C₄H₉ | C₂H₅ |
| A-168 | CH₂CH═CH₂ | t-C₄H₉ | C₂H₅ |
| A-169 | CH₂Ph | t-C₄H₉ | C₂H₅ |
| A-170 | Ph | t-C₄H₉ | C₂H₅ |
| A-171 | H | CH₂Ph | C₂H₅ |
| A-172 | CH₃ | CH₂Ph | C₂H₅ |
| A-173 | CH₂CH═CH₂ | CH₂Ph | C₂H₅ |
| A-174 | CH₂Ph | CH₂Ph | C₂H₅ |
| A-175 | Ph | CH₂Ph | C₂H₅ |
| A-176 | H | Ph | C₂H₅ |
| A-177 | CH₃ | Ph | C₂H₅ |
| A-178 | CH₂CH═CH₂ | Ph | C₂H₅ |
| A-179 | CH₂Ph | Ph | C₂H₅ |
| A-180 | Ph | Ph | C₂H₅ |

| | | |
|---|---|---|
| A-181 | H | CH₂CH₂ |
| A-182 | CH₃ | CH₂CH₂ |
| A-183 | CH₂CH═CH₂ | CH₂CH₂ |
| A-184 | CH₂Ph | CH₂CH₂ |
| A-185 | Ph | CH₂CH₂ |
| A-186 | H | CH₂H₂CH₂ |
| A-187 | CH₃ | CH₂H₂CH₂ |
| A-188 | CH₂CH═CH₂ | CH₂H₂CH₂ |
| A-189 | CH₂Ph | CH₂H₂CH₂ |
| A-190 | Ph | CH₂H₂CH₂ |
| A-191 | H | CH₂CH₂CH₂CH₂ |
| A-192 | CH₃ | CH₂CH₂CH₂CH₂ |
| A-193 | CH₂CH═CH₂ | CH₂CH₂CH₂CH₂ |
| A-194 | CH₂Ph | CH₂CH₂CH₂CH₂ |
| A-195 | Ph | CH₂CH₂CH₂CH₂ |
| A-196 | H | CH₂CH₂CH₂CH₂CH₂ |
| A-197 | CH₃ | CH₂CH₂CH₂CH₂CH₂ |
| A-198 | CH₂CH═CH₂ | CH₂CH₂CH₂CH₂CH₂ |
| A-199 | CH₂Ph | CH₂CH₂CH₂CH₂CH₂ |
| A-200 | Ph | CH₂CH₂CH₂CH₂CH₂ |

TABLE 2
Compounds of formula (Ib):

| | |
|---|---|
| | (Ib) |
| 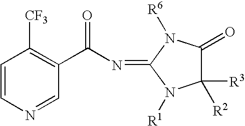 | |

| Com- | | | | |
|---|---|---|---|---|
| pound | R¹ | R² | R³ | R⁶ |
| B-1 | H | H | H | H |
| B-2 | CH₃ | H | H | H |
| B-3 | CH₂CH═CH₂ | H | H | H |
| B-4 | CH₂Ph | H | H | H |
| B-5 | Ph | H | H | H |
| B-6 | H | CH₃ | H | H |
| B-7 | CH₃ | CH₃ | H | H |
| B-8 | CH₂CH═CH₂ | CH₃ | H | H |
| B-9 | CH₂Ph | CH₃ | H | H |
| B-10 | Ph | CH₃ | H | H |
| B-11 | H | CH₃CH₂ | H | H |
| B-12 | CH₃ | CH₃CH₂ | H | H |
| B-13 | CH₂CH═CH₂ | CH₃CH₂ | H | H |
| B-14 | CH₂Ph | CH₃CH₂ | H | H |
| B-15 | Ph | CH₃CH₂ | H | H |
| B-16 | H | n-C₃H₇ | H | H |
| B-17 | CH₃ | n-C₃H₇ | H | H |
| B-18 | CH₂CH═CH₂ | n-C₃H₇ | H | H |
| B-19 | CH₂Ph | n-C₃H₇ | H | H |
| B-20 | Ph | n-C₃H₇ | H | H |
| B-21 | H | i-C₃H₇ | H | H |
| B-22 | CH₃ | i-C₃H₇ | H | H |
| B-23 | CH₂CH═CH₂ | i-C₃H₇ | H | H |
| B-24 | CH₂Ph | i-C₃H₇ | H | H |
| B-25 | Ph | i-C₃H₇ | H | H |
| B-26 | H | n-C₄H₉ | H | H |
| B-27 | CH₃ | n-C₄H₉ | H | H |
| B-28 | CH₂CH═CH₂ | n-C₄H₉ | H | H |
| B-29 | CH₂Ph | n-C₄H₉ | H | H |
| B-30 | Ph | n-C₄H₉ | H | H |
| B-31 | H | i-C₄H₉ | H | H |
| B-32 | CH₃ | i-C₄H₉ | H | H |
| B-33 | CH₂CH═CH₂ | i-C₄H₉ | H | H |
| B-34 | CH₂Ph | i-C₄H₉ | H | H |
| B-35 | Ph | i-C₄H₉ | H | H |
| B-36 | H | s-C₄H₉ | H | H |
| B-37 | CH₃ | s-C₄H₉ | H | H |
| B-38 | CH₂CH═CH₂ | s-C₄H₉ | H | H |
| B-39 | CH₂Ph | s-C₄H₉ | H | H |
| B-40 | Ph | s-C₄H₉ | H | H |
| B-41 | H | t-C₄H₉ | H | H |
| B-42 | CH₃ | t-C₄H₉ | H | H |
| B-43 | CH₂CH═CH₂ | t-C₄H₉ | H | H |
| B-44 | CH₂Ph | t-C₄H₉ | H | H |
| B-45 | Ph | t-C₄H₉ | H | H |
| B-46 | H | CH₂Ph | H | H |
| B-47 | CH₃ | CH₂Ph | H | H |
| B-48 | CH₂CH═CH₂ | CH₂Ph | H | H |
| B-49 | CH₂Ph | CH₂Ph | H | H |
| B-50 | Ph | CH₂Ph | H | H |
| B-51 | H | Ph | H | H |
| B-52 | CH₃ | Ph | H | H |
| B-53 | CH₂CH═CH₂ | Ph | H | H |
| B-54 | CH₂Ph | Ph | H | H |
| B-55 | Ph | Ph | H | H |
| B-56 | H | CH₂OH | H | H |
| B-57 | CH₃ | CH₂OH | H | H |
| B-58 | CH₂CH═CH₂ | CH₂OH | H | H |
| B-59 | CH₂Ph | CH₂OH | H | H |
| B-60 | Ph | CH₂OH | H | H |
| B-61 | H | CH(OH)CH₃ | H | H |
| B-62 | CH₃ | CH(OH)CH₃ | H | H |
| B-63 | CH₂CH═CH₂ | CH(OH)CH₃ | H | H |
| B-64 | CH₂Ph | CH(OH)CH₃ | H | H |
| B-65 | Ph | CH(OH)CH₃ | H | H |
| B-66 | H | CH₂SH | H | H |
| B-67 | CH₃ | CH₂SH | H | H |
| B-68 | CH₂CH═CH₂ | CH₂SH | H | H |
| B-69 | CH₂Ph | CH₂SH | H | H |
| B-70 | Ph | CH₂SH | H | H |
| B-71 | H | CH₂SCH₂CH₃ | H | H |
| B-72 | CH₃ | CH₂SCH₂CH₃ | H | H |
| B-73 | CH₂CH═CH₂ | CH₂SCH₂CH₃ | H | H |
| B-74 | CH₂Ph | CH₂SCH₂CH₃ | H | H |
| B-75 | Ph | CH₂SCH₂CH₃ | H | H |
| B-76 | H | CH₂CONH₂ | H | H |
| B-77 | CH₃ | CH₂CONH₂ | H | H |
| B-78 | CH₂CH═CH₂ | CH₂CONH₂ | H | H |
| B-79 | CH₂Ph | CH₂CONH₂ | H | H |
| B-80 | Ph | CH₂CONH₂ | H | H |
| B-81 | H | CH₂CH₂CONH₂ | H | H |
| B-82 | CH₃ | CH₂CH₂CONH₂ | H | H |
| B-83 | CH₂CH═CH₂ | CH₂CH₂CONH₂ | H | H |
| B-84 | CH₂Ph | CH₂CH₂CONH₂ | H | H |
| B-85 | Ph | CH₂CH₂CONH₂ | H | H |
| B-86 | H | CH₃ | CH₃ | H |
| B-87 | CH₃ | CH₃ | CH₃ | H |
| B-88 | CH₂CH═CH₂ | CH₃ | CH₃ | H |
| B-89 | CH₂Ph | CH₃ | CH₃ | H |
| B-90 | Ph | CH₃ | CH₃ | H |
| B-91 | H | CH₃CH₂ | CH₃ | H |
| B-92 | CH₃ | CH₃CH₂ | CH₃ | H |
| B-93 | CH₂CH═CH₂ | CH₃CH₂ | CH₃ | H |
| B-94 | CH₂Ph | CH₃CH₂ | CH₃ | H |
| B-95 | Ph | CH₃CH₂ | CH₃ | H |
| B-96 | H | n-C₃H₇ | CH₃ | H |
| B-97 | CH₃ | n-C₃H₇ | CH₃ | H |
| B-98 | CH₂CH═CH₂ | n-C₃H₇ | CH₃ | H |
| B-99 | CH₂Ph | n-C₃H₇ | CH₃ | H |
| B-100 | Ph | n-C₃H₇ | CH₃ | H |
| B-101 | H | i-C₃H₇ | CH₃ | H |
| B-102 | CH₃ | i-C₃H₇ | CH₃ | H |
| B-103 | CH₂CH═CH₂ | i-C₃H₇ | CH₃ | H |
| B-104 | CH₂Ph | i-C₃H₇ | CH₃ | H |
| B-105 | Ph | i-C₃H₇ | CH₃ | H |
| B-106 | H | n-C₄H₉ | CH₃ | H |
| B-107 | CH₃ | n-C₄H₉ | CH₃ | H |
| B-108 | CH₂CH═CH₂ | n-C₄H₉ | CH₃ | H |
| B-109 | CH₂Ph | n-C₄H₉ | CH₃ | H |
| B-110 | Ph | n-C₄H₉ | CH₃ | H |
| B-111 | H | i-C₄H₉ | CH₃ | H |
| B-112 | CH₃ | i-C₄H₉ | CH₃ | H |
| B-113 | CH₂CH═CH₂ | i-C₄H₉ | CH₃ | H |
| B-114 | CH₂Ph | i-C₄H₉ | CH₃ | H |
| B-115 | Ph | i-C₄H₉ | CH₃ | H |
| B-116 | H | s-C₄H₉ | CH₃ | H |
| B-117 | CH₃ | s-C₄H₉ | CH₃ | H |
| B-118 | CH₂CH═CH₂ | s-C₄H₉ | CH₃ | H |
| B-119 | CH₂Ph | s-C₄H₉ | CH₃ | H |
| B-120 | Ph | s-C₄H₉ | CH₃ | H |
| B-121 | H | t-C₄H₉ | CH₃ | H |
| B-122 | CH₃ | t-C₄H₉ | CH₃ | H |
| B-123 | CH₂CH═CH₂ | t-C₄H₉ | CH₃ | H |
| B-124 | CH₂Ph | t-C₄H₉ | CH₃ | H |
| B-125 | Ph | t-C₄H₉ | CH₃ | H |
| B-126 | H | CH₂Ph | CH₃ | H |
| B-127 | CH₃ | CH₂Ph | CH₃ | H |
| B-128 | CH₂CH═CH₂ | CH₂Ph | CH₃ | H |
| B-129 | CH₂Ph | CH₂Ph | CH₃ | H |
| B-130 | Ph | CH₂Ph | CH₃ | H |
| B-131 | H | Ph | CH₃ | H |
| B-132 | CH₃ | Ph | CH₃ | H |
| B-133 | CH₂CH═CH₂ | Ph | CH₃ | H |
| B-134 | CH₂Ph | Ph | CH₃ | H |
| B-135 | Ph | Ph | CH₃ | H |
| B-136 | H | C₂H₅ | C₂H₅ | H |
| B-137 | CH₃ | C₂H₅ | C₂H₅ | H |
| B-138 | CH₂CH═CH₂ | C₂H₅ | C₂H₅ | H |
| B-139 | CH₂Ph | C₂H₅ | C₂H₅ | H |
| B-140 | Ph | C₂H₅ | C₂H₅ | H |
| B-141 | H | n-C₃H₇ | C₂H₅ | H |
| B-142 | CH₃ | n-C₃H₇ | C₂H₅ | H |
| B-143 | CH₂CH═CH₂ | n-C₃H₇ | C₂H₅ | H |
| B-144 | CH₂Ph | n-C₃H₇ | C₂H₅ | H |
| B-145 | Ph | n-C₃H₇ | C₂H₅ | H |
| B-146 | H | i-C₃H₇ | C₂H₅ | H |
| B-147 | CH₃ | i-C₃H₇ | C₂H₅ | H |
| B-148 | CH₂CH═CH₂ | i-C₃H₇ | C₂H₅ | H |
| B-149 | CH₂Ph | i-C₃H₇ | C₂H₅ | H |
| B-150 | Ph | i-C₃H₇ | C₂H₅ | H |
| B-151 | H | n-C₄H₉ | C₂H₅ | H |
| B-152 | CH₃ | n-C₄H₉ | C₂H₅ | H |
| B-153 | CH₂CH═CH₂ | n-C₄H₉ | C₂H₅ | H |
| B-154 | CH₂Ph | n-C₄H₉ | C₂H₅ | H |
| B-155 | Ph | n-C₄H₉ | C₂H₅ | H |
| B-156 | H | i-C₄H₉ | C₂H₅ | H |
| B-157 | CH₃ | i-C₄H₉ | C₂H₅ | H |
| B-158 | CH₂CH═CH₂ | i-C₄H₉ | C₂H₅ | H |
| B-159 | CH₂Ph | i-C₄H₉ | C₂H₅ | H |
| B-160 | Ph | i-C₄H₉ | C₂H₅ | H |
| B-161 | H | s-C₄H₉ | C₂H₅ | H |
| B-162 | CH₃ | s-C₄H₉ | C₂H₅ | H |
| B-163 | CH₂CH═CH₂ | s-C₄H₉ | C₂H₅ | H |
| B-164 | CH₂Ph | s-C₄H₉ | C₂H₅ | H |
| B-165 | Ph | s-C₄H₉ | C₂H₅ | H |
| B-166 | H | t-C₄H₉ | C₂H₅ | H |
| B-167 | CH₃ | t-C₄H₉ | C₂H₅ | H |
| B-168 | CH₂CH═CH₂ | t-C₄H₉ | C₂H₅ | H |
| B-169 | CH₂Ph | t-C₄H₉ | C₂H₅ | H |
| B-170 | Ph | t-C₄H₉ | C₂H₅ | H |
| B-171 | H | CH₂Ph | C₂H₅ | H |
| B-172 | CH₃ | CH₂Ph | C₂H₅ | H |
| B-173 | CH₂CH═CH₂ | CH₂Ph | C₂H₅ | H |
| B-174 | CH₂Ph | CH₂Ph | C₂H₅ | H |
| B-175 | Ph | CH₂Ph | C₂H₅ | H |
| B-176 | H | Ph | C₂H₅ | H |
| B-177 | CH₃ | Ph | C₂H₅ | H |
| B-178 | CH₂CH═CH₂ | Ph | C₂H₅ | H |
| B-179 | CH₂Ph | Ph | C₂H₅ | H |
| B-180 | Ph | Ph | C₂H₅ | H |
| B-181 | H | CH₂CH₂ | | H |
| B-182 | CH₃ | CH₂CH₂ | | H |
| B-183 | CH₂CH═CH₂ | CH₂CH₂ | | H |
| B-184 | CH₂Ph | CH₂CH₂ | | H |
| B-185 | Ph | CH₂CH₂ | | H |
| B-186 | H | CH₂H₂CH₂ | | H |
| B-187 | CH₃ | CH₂H₂CH₂ | | H |
| B-188 | CH₂CH═CH₂ | CH₂H₂CH₂ | | H |
| B-189 | CH₂Ph | CH₂H₂CH₂ | | H |
| B-190 | Ph | CH₂H₂CH₂ | | H |
| B-191 | H | CH₂CH₂CH₂CH₂ | | H |
| B-192 | CH₃ | CH₂CH₂CH₂CH₂ | | H |
| B-193 | CH₂CH═CH₂ | CH₂CH₂CH₂CH₂ | | H |
| B-194 | CH₂Ph | CH₂CH₂CH₂CH₂ | | H |
| B-195 | Ph | CH₂CH₂CH₂CH₂ | | H |
| B-196 | H | CH₂CH₂CH₂CH₂CH₂ | | H |
| B-197 | CH₃ | CH₂CH₂CH₂CH₂CH₂ | | H |
| B-198 | CH₂CH═CH₂ | CH₂CH₂CH₂CH₂CH₂ | | H |
| B-199 | CH₂Ph | CH₂CH₂CH₂CH₂CH₂ | | H |
| B-200 | Ph | CH₂CH₂CH₂CH₂CH₂ | | H |
| B-201 | H | H | H | CH₃ |
| B-202 | CH₃ | H | H | CH₃ |
| B-203 | CH₂CH═CH₂ | H | H | CH₃ |
| B-204 | CH₂Ph | H | H | CH₃ |
| B-205 | Ph | H | H | CH₃ |
| B-206 | H | CH₃ | H | CH₃ |
| B-207 | CH₃ | CH₃ | H | CH₃ |
| B-208 | CH₂CH═CH₂ | CH₃ | H | CH₃ |
| B-209 | CH₂Ph | CH₃ | H | CH₃ |
| B-210 | Ph | CH₃ | H | CH₃ |
| B-211 | H | CH₃CH₂ | H | CH₃ |
| B-212 | CH₃ | CH₃CH₂ | H | CH₃ |
| B-213 | CH₂CH═CH₂ | CH₃CH₂ | H | CH₃ |
| B-214 | CH₂Ph | CH₃CH₂ | H | CH₃ |
| B-215 | Ph | CH₃CH₂ | H | CH₃ |
| B-216 | H | n-C₃H₇ | H | CH₃ |
| B-217 | CH₃ | n-C₃H₇ | H | CH₃ |
| B-218 | CH₂CH═CH₂ | n-C₃H₇ | H | CH₃ |
| B-219 | CH₂Ph | n-C₃H₇ | H | CH₃ |
| B-220 | Ph | n-C₃H₇ | H | CH₃ |
| B-221 | H | i-C₃H₇ | H | CH₃ |
| B-222 | CH₃ | i-C₃H₇ | H | CH₃ |
| B-223 | CH₂CH═CH₂ | i-C₃H₇ | H | CH₃ |
| B-224 | CH₂Ph | i-C₃H₇ | H | CH₃ |
| B-225 | Ph | i-C₃H₇ | H | CH₃ |
| B-226 | H | n-C₄H₉ | H | CH₃ |
| B-227 | CH₃ | n-C₄H₉ | H | CH₃ |
| B-228 | CH₂CH═CH₂ | n-C₄H₉ | H | CH₃ |
| B-229 | CH₂Ph | n-C₄H₉ | H | CH₃ |
| B-230 | Ph | n-C₄H₉ | H | CH₃ |
| B-231 | H | i-C₄H₉ | H | CH₃ |
| B-232 | CH₃ | i-C₄H₉ | H | CH₃ |
| B-233 | CH₂CH═CH₂ | i-C₄H₉ | H | CH₃ |
| B-234 | CH₂Ph | i-C₄H₉ | H | CH₃ |
| B-235 | Ph | i-C₄H₉ | H | CH₃ |
| B-236 | H | s-C₄H₉ | H | CH₃ |
| B-237 | CH₃ | s-C₄H₉ | H | CH₃ |
| B-238 | CH₂CH═CH₂ | s-C₄H₉ | H | CH₃ |
| B-239 | CH₂Ph | s-C₄H₉ | H | CH₃ |
| B-240 | Ph | s-C₄H₉ | H | CH₃ |
| B-241 | H | t-C₄H₉ | H | CH₃ |
| B-242 | CH₃ | t-C₄H₉ | H | CH₃ |
| B-243 | CH₂CH═CH₂ | t-C₄H₉ | H | CH₃ |
| B-244 | CH₂Ph | t-C₄H₉ | H | CH₃ |
| B-245 | Ph | t-C₄H₉ | H | CH₃ |
| B-246 | H | CH₂Ph | H | CH₃ |
| B-247 | CH₃ | CH₂Ph | H | CH₃ |
| B-248 | CH₂CH═CH₂ | CH₂Ph | H | CH₃ |
| B-249 | CH₂Ph | CH₂Ph | H | CH₃ |
| B-250 | Ph | CH₂Ph | H | CH₃ |
| B-251 | H | Ph | H | CH₃ |
| B-252 | CH₃ | Ph | H | CH₃ |
| B-253 | CH₂CH═CH₂ | Ph | H | CH₃ |
| B-254 | CH₂Ph | Ph | H | CH₃ |
| B-255 | Ph | Ph | H | CH₃ |
| B-256 | H | CH₂OH | H | CH₃ |
| B-257 | CH₃ | CH₂OH | H | CH₃ |
| B-258 | CH₂CH═CH₂ | CH₂OH | H | CH₃ |
| B-259 | CH₂Ph | CH₂OH | H | CH₃ |
| B-260 | Ph | CH₂OH | H | CH₃ |
| B-261 | H | CH(OH)CH₃ | H | CH₃ |
| B-262 | CH₃ | CH(OH)CH₃ | H | CH₃ |
| B-263 | CH₂CH═CH₂ | CH(OH)CH₃ | H | CH₃ |
| B-264 | CH₂Ph | CH(OH)CH₃ | H | CH₃ |
| B-265 | Ph | CH(OH)CH₃ | H | CH₃ |
| B-266 | H | CH₂SH | H | CH₃ |
| B-267 | CH₃ | CH₂SH | H | CH₃ |
| B-268 | CH₂CH═CH₂ | CH₂SH | H | CH₃ |
| B-269 | CH₂Ph | CH₂SH | H | CH₃ |
| B-270 | Ph | CH₂SH | H | CH₃ |
| B-271 | H | CH₂SCH₂CH₃ | H | CH₃ |
| B-272 | CH₃ | CH₂SCH₂CH₃ | H | CH₃ |
| B-273 | CH₂CH═CH₂ | CH₂SCH₂CH₃ | H | CH₃ |
| B-274 | CH₂Ph | CH₂SCH₂CH₃ | H | CH₃ |
| B-275 | Ph | CH₂SCH₂CH₃ | H | CH₃ |
| B-276 | H | CH₂CONH₂ | H | CH₃ |
| B-277 | CH₃ | CH₂CONH₂ | H | CH₃ |
| B-278 | CH₂CH═CH₂ | CH₂CONH₂ | H | CH₃ |
| B-279 | CH₂Ph | CH₂CONH₂ | H | CH₃ |
| B-280 | Ph | CH₂CONH₂ | H | CH₃ |
| B-281 | H | CH₂CH₂CONH₂ | H | CH₃ |
| B-282 | CH₃ | CH₂CH₂CONH₂ | H | CH₃ |
| B-283 | CH₂CH═CH₂ | CH₂CH₂CONH₂ | H | CH₃ |
| B-284 | CH₂Ph | CH₂CH₂CONH₂ | H | CH₃ |
| B-285 | Ph | CH₂CH₂CONH₂ | H | CH₃ |
| B-286 | H | CH₃ | CH₃ | CH₃ |
| B-287 | CH₃ | CH₃ | CH₃ | CH₃ |
| B-288 | CH₂CH═CH₂ | CH₃ | CH₃ | CH₃ |
| B-289 | CH₂Ph | CH₃ | CH₃ | CH₃ |
| B-290 | Ph | CH₃ | CH₃ | CH₃ |
| B-291 | H | CH₃CH₂ | CH₃ | CH₃ |
| B-292 | CH₃ | CH₃CH₂ | CH₃ | CH₃ |
| B-293 | CH₂CH═CH₂ | CH₃CH₂ | CH₃ | CH₃ |
| B-294 | CH₂Ph | CH₃CH₂ | CH₃ | CH₃ |
| B-295 | Ph | CH₃CH₂ | CH₃ | CH₃ |
| B-296 | H | n-C₃H₇ | CH₃ | CH₃ |
| B-297 | CH₃ | n-C₃H₇ | CH₃ | CH₃ |
| B-298 | CH₂CH═CH₂ | n-C₃H₇ | CH₃ | CH₃ |
| B-299 | CH₂Ph | n-C₃H₇ | CH₃ | CH₃ |
| B-300 | Ph | n-C₃H₇ | CH₃ | CH₃ |
| B-301 | H | i-C₃H₇ | CH₃ | CH₃ |
| B-302 | CH₃ | i-C₃H₇ | CH₃ | CH₃ |
| B-303 | CH₂CH═CH₂ | i-C₃H₇ | CH₃ | CH₃ |
| B-304 | CH₂Ph | i-C₃H₇ | CH₃ | CH₃ |
| B-305 | Ph | i-C₃H₇ | CH₃ | CH₃ |
| B-306 | H | n-C₄H₉ | CH₃ | CH₃ |
| B-307 | CH₃ | n-C₄H₉ | CH₃ | CH₃ |
| B-308 | CH₂CH═CH₂ | n-C₄H₉ | CH₃ | CH₃ |
| B-309 | CH₂Ph | n-C₄H₉ | CH₃ | CH₃ |
| B-310 | Ph | n-C₄H₉ | CH₃ | CH₃ |
| B-311 | H | i-C₄H₉ | CH₃ | CH₃ |
| B-312 | CH₃ | i-C₄H₉ | CH₃ | CH₃ |
| B-313 | CH₂CH═CH₂ | i-C₄H₉ | CH₃ | CH₃ |
| B-314 | CH₂Ph | i-C₄H₉ | CH₃ | CH₃ |
| B-315 | Ph | i-C₄H₉ | CH₃ | CH₃ |
| B-316 | H | s-C₄H₉ | CH₃ | CH₃ |
| B-317 | CH₃ | s-C₄H₉ | CH₃ | CH₃ |
| B-318 | CH₂CH═CH₂ | s-C₄H₉ | CH₃ | CH₃ |
| B-319 | CH₂Ph | s-C₄H₉ | CH₃ | CH₃ |
| B-320 | Ph | s-C₄H₉ | CH₃ | CH₃ |
| B-321 | H | t-C₄H₉ | CH₃ | CH₃ |
| B-322 | CH₃ | t-C₄H₉ | CH₃ | CH₃ |
| B-323 | CH₂CH═CH₂ | t-C₄H₉ | CH₃ | CH₃ |
| B-324 | CH₂Ph | t-C₄H₉ | CH₃ | CH₃ |
| B-325 | Ph | t-C₄H₉ | CH₃ | CH₃ |
| B-326 | H | CH₂Ph | CH₃ | CH₃ |
| B-327 | CH₃ | CH₂Ph | CH₃ | CH₃ |
| B-328 | CH₂CH═CH₂ | CH₂Ph | CH₃ | CH₃ |
| B-329 | CH₂Ph | CH₂Ph | CH₃ | CH₃ |
| B-330 | Ph | CH₂Ph | CH₃ | CH₃ |
| B-331 | H | Ph | CH₃ | CH₃ |
| B-332 | CH₃ | Ph | CH₃ | CH₃ |
| B-333 | CH₂CH═CH₂ | Ph | CH₃ | CH₃ |
| B-334 | CH₂Ph | Ph | CH₃ | CH₃ |
| B-335 | Ph | Ph | CH₃ | CH₃ |
| B-336 | H | CH₂CH₂ | | CH₃ |
| B-337 | CH₃ | CH₂CH₂ | | CH₃ |
| B-338 | CH₂CH═CH₂ | CH₂CH₂ | | CH₃ |
| B-339 | CH₂Ph | CH₂CH₂ | | CH₃ |
| B-340 | Ph | CH₂CH₂ | | CH₃ |
| B-341 | H | CH₂H₂CH₂ | | CH₃ |
| B-342 | CH₃ | CH₂H₂CH₂ | | CH₃ |
| B-343 | CH₂CH═CH₂ | CH₂H₂CH₂ | | CH₃ |
| B-344 | CH₂Ph | CH₂H₂CH₂ | | CH₃ |
| B-345 | Ph | CH₂H₂CH₂ | | CH₃ |
| B-346 | H | CH₂CH₂CH₂CH₂ | | CH₃ |
| B-347 | CH₃ | CH₂CH₂CH₂CH₂ | | CH₃ |
| B-348 | CH₂CH═CH₂ | CH₂CH₂CH₂CH₂ | | CH₃ |
| B-349 | CH₂Ph | CH₂CH₂CH₂CH₂ | | CH₃ |
| B-350 | Ph | CH₂CH₂CH₂CH₂ | | CH₃ |
| B-351 | H | CH₂CH₂CH₂CH₂CH₂ | | CH₃ |
| B-352 | CH₃ | CH₂CH₂CH₂CH₂CH₂ | | CH₃ |
| B-353 | CH₂CH═CH₂ | CH₂CH₂CH₂CH₂CH₂ | | CH₃ |
| B-354 | CH₂Ph | CH₂CH₂CH₂CH₂CH₂ | | CH₃ |
| B-355 | Ph | CH₂CH₂CH₂CH₂CH₂ | | CH₃ |
| B-356 | H | H | H | CH₃CH₂ |
| B-357 | H | CH₃ | H | CH₃CH₂ |
| B-358 | H | CH₃CH₂ | H | CH₃CH₂ |
| B-359 | H | n-C₃H₇ | H | CH₃CH₂ |
| B-360 | H | i-C₃H₇ | H | CH₃CH₂ |
| B-361 | H | n-C₄H₉ | H | CH₃CH₂ |
| B-362 | H | i-C₄H₉ | H | CH₃CH₂ |
| B-363 | H | s-C₄H₉ | H | CH₃CH₂ |
| B-364 | H | t-C₄H₉ | H | CH₃CH₂ |
| B-365 | H | CH₂Ph | H | CH₃CH₂ |
| B-366 | H | Ph | H | CH₃CH₂ |
| B-367 | H | CH₂OH | H | CH₃CH₂ |
| B-368 | H | CH(OH)CH₃ | H | CH₃CH₂ |
| B-369 | H | CH₂SH | H | CH₃CH₂ |
| B-370 | H | CH₂SCH₂CH₃ | H | CH₃CH₂ |
| B-371 | H | CH₂CONH₂ | H | CH₃CH₂ |
| B-372 | H | CH₂CH₂CONH₂ | H | CH₃CH₂ |
| B-373 | H | CH₃ | CH₃ | CH₃CH₂ |
| B-374 | H | CH₃CH₂ | CH₃ | CH₃CH₂ |
| B-375 | H | n-C₃H₇ | CH₃ | CH₃CH₂ |
| B-376 | H | i-C₃H₇ | CH₃ | CH₃CH₂ |
| B-377 | H | n-C₄H₉ | CH₃ | CH₃CH₂ |
| B-378 | H | i-C₄H₉ | CH₃ | CH₃CH₂ |
| B-379 | H | s-C₄H₉ | CH₃ | CH₃CH₂ |
| B-380 | H | t-C₄H₉ | CH₃ | CH₃CH₂ |
| B-381 | H | CH₂Ph | CH₃ | CH₃CH₂ |
| B-382 | H | Ph | CH₃ | CH₃CH₂ |
| B-383 | H | CH₂OH | CH₃ | CH₃CH₂ |
| B-384 | H | CH(OH)CH₃ | CH₃ | CH₃CH₂ |
| B-385 | H | CH₂SH | CH₃ | CH₃CH₂ |
| B-386 | H | CH₂SCH₂CH₃ | CH₃ | CH₃CH₂ |
| B-387 | H | CH₂CONH₂ | CH₃ | CH₃CH₂ |
| B-388 | H | CH₂CH₂CONH₂ | CH₃ | CH₃CH₂ |
| B-389 | H | H | H | i-C₃H₇ |
| B-390 | H | CH₃ | H | i-C₃H₇ |
| B-391 | H | CH₃CH₂ | H | i-C₃H₇ |
| B-392 | H | n-C₃H₇ | H | i-C₃H₇ |
| B-393 | H | i-C₃H₇ | H | i-C₃H₇ |
| B-394 | H | n-C₄H₉ | H | i-C₃H₇ |
| B-395 | H | i-C₄H₉ | H | i-C₃H₇ |
| B-396 | H | s-C₄H₉ | H | i-C₃H₇ |
| B-397 | H | t-C₄H₉ | H | i-C₃H₇ |
| B-398 | H | CH₂Ph | H | i-C₃H₇ |
| B-399 | H | Ph | H | i-C₃H₇ |
| B-400 | H | CH₂OH | H | i-C₃H₇ |
| B-401 | H | CH(OH)CH₃ | H | i-C₃H₇ |
| B-402 | H | CH₂SH | H | i-C₃H₇ |
| B-403 | H | CH₂SCH₂CH₃ | H | i-C₃H₇ |
| B-404 | H | CH₂CONH₂ | H | i-C₃H₇ |
| B-405 | H | CH₂CH₂CONH₂ | H | i-C₃H₇ |
| B-406 | H | CH₃ | H | i-C₃H₇ |
| B-407 | H | CH₃CH₂ | CH₃ | i-C₃H₇ |
| B-408 | H | n-C₃H₇ | CH₃ | i-C₃H₇ |
| B-409 | H | i-C₃H₇ | CH₃ | i-C₃H₇ |
| B-410 | H | n-C₄H₉ | CH₃ | i-C₃H₇ |
| B-411 | H | i-C₄H₉ | CH₃ | i-C₃H₇ |
| B-412 | H | s-C₄H₉ | CH₃ | i-C₃H₇ |
| B-413 | H | t-C₄H₉ | CH₃ | i-C₃H₇ |
| B-414 | H | CH₂Ph | CH₃ | i-C₃H₇ |
| B-415 | H | Ph | CH₃ | i-C₃H₇ |
| B-416 | H | CH₂OH | CH₃ | i-C₃H₇ |
| B-417 | H | CH(OH)CH₃ | CH₃ | i-C₃H₇ |
| B-418 | H | CH₂SH | CH₃ | i-C₃H₇ |
| B-419 | H | CH₂SCH₂CH₃ | CH₃ | i-C₃H₇ |
| B-420 | H | CH₂CONH₂ | CH₃ | i-C₃H₇ |
| B-421 | H | CH₂CH₂CONH₂ | CH₃ | i-C₃H₇ |
| B-422 | H | H | H | t-C₄H₉ |
| B-423 | H | CH₃ | H | t-C₄H₉ |
| B-424 | H | CH₃CH₂ | H | t-C₄H₉ |
| B-425 | H | n-C₃H₇ | H | t-C₄H₉ |
| B-426 | H | i-C₃H₇ | H | t-C₄H₉ |
| B-427 | H | n-C₄H₉ | H | t-C₄H₉ |
| B-428 | H | i-C₄H₉ | H | t-C₄H₉ |
| B-429 | H | s-C₄H₉ | H | t-C₄H₉ |
| B-430 | H | t-C₄H₉ | H | t-C₄H₉ |
| B-431 | H | CH₂Ph | H | t-C₄H₉ |
| B-432 | H | Ph | H | t-C₄H₉ |
| B-433 | H | CH₂OH | H | t-C₄H₉ |
| B-434 | H | CH(OH)CH₃ | H | t-C₄H₉ |
| B-435 | H | CH₂SH | H | t-C₄H₉ |
| B-436 | H | CH₂SCH₂CH₃ | H | t-C₄H₉ |
| B-437 | H | CH₂CONH₂ | H | t-C₄H₉ |
| B-438 | H | CH₂CH₂CONH₂ | H | t-C₄H₉ |
| B-439 | H | CH₃ | CH₃ | t-C₄H₉ |
| B-440 | H | CH₃CH₂ | CH₃ | t-C₄H₉ |
| B-441 | H | n-C₃H₇ | CH₃ | t-C₄H₉ |
| B-442 | H | i-C₃H₇ | CH₃ | t-C₄H₉ |
| B-443 | H | n-C₄H₉ | CH₃ | t-C₄H₉ |
| B-444 | H | i-C₄H₉ | CH₃ | t-C₄H₉ |
| B-445 | H | s-C₄H₉ | CH₃ | t-C₄H₉ |
| B-446 | H | t-C₄H₉ | CH₃ | t-C₄H₉ |
| B-447 | H | CH₂Ph | CH₃ | t-C₄H₉ |
| B-448 | H | Ph | CH₃ | t-C₄H₉ |
| B-449 | H | CH₂OH | CH₃ | t-C₄H₉ |
| B-450 | H | CH(OH)CH₃ | CH₃ | t-C₄H₉ |
| B-451 | H | CH₂SH | CH₃ | t-C₄H₉ |
| B-452 | H | CH₂SCH₂CH₃ | CH₃ | t-C₄H₉ |
| B-453 | H | CH₂CONH₂ | CH₃ | t-C₄H₉ |
| B-454 | H | CH₂CH₂CONH₂ | CH₃ | t-C₄H₉ |
| B-455 | H | H | H | CH₂CH═CH₂ |
| B-456 | H | CH₃ | H | CH₂CH═CH₂ |
| B-457 | H | CH₃CH₂ | H | CH₂CH═CH₂ |
| B-458 | H | n-C₃H₇ | H | CH₂CH═CH₂ |
| B-459 | H | i-C₃H₇ | H | CH₂CH═CH₂ |
| B-460 | H | n-C₄H₉ | H | CH₂CH═CH₂ |
| B-461 | H | i-C₄H₉ | H | CH₂CH═CH₂ |
| B-462 | H | s-C₄H₉ | H | CH₂CH═CH₂ |
| B-463 | H | t-C₄H₉ | H | CH₂CH═CH₂ |
| B-464 | H | CH₂Ph | H | CH₂CH═CH₂ |
| B-465 | H | Ph | H | CH₂CH═CH₂ |
| B-466 | H | CH₂OH | H | CH₂CH═CH₂ |
| B-467 | H | CH(OH)CH₃ | H | CH₂CH═CH₂ |
| B-468 | H | CH₂SH | H | CH₂CH═CH₂ |
| B-469 | H | CH₂SCH₂CH₃ | H | CH₂CH═CH₂ |
| B-470 | H | CH₂CONH₂ | H | CH₂CH═CH₂ |
| B-471 | H | CH₂CH₂CONH₂ | H | CH₂CH═CH₂ |
| B-472 | H | CH₃ | CH₃ | CH₂CH═CH₂ |
| B-473 | H | CH₃CH₂ | CH₃ | CH₂CH═CH₂ |
| B-474 | H | n-C₃H₇ | CH₃ | CH₂CH═CH₂ |
| B-475 | H | i-C₃H₇ | CH₃ | CH₂CH═CH₂ |
| B-476 | H | n-C₄H₉ | CH₃ | CH₂CH═CH₂ |
| B-477 | H | i-C₄H₉ | CH₃ | CH₂CH═CH₂ |
| B-478 | H | s-C₄H₉ | CH₃ | CH₂CH═CH₂ |
| B-479 | H | t-C₄H₉ | CH₃ | CH₂CH═CH₂ |
| B-480 | H | CH₂Ph | CH₃ | CH₂CH═CH₂ |
| B-481 | H | Ph | CH₃ | CH₂CH═CH₂ |
| B-482 | H | CH₂OH | CH₃ | CH₂CH═CH₂ |
| B-483 | H | CH(OH)CH₃ | CH₃ | CH₂CH═CH₂ |
| B-484 | H | CH₂SH | CH₃ | CH₂CH═CH₂ |
| B-485 | H | CH₂SCH₂CH₃ | CH₃ | CH₂CH═CH₂ |
| B-486 | H | CH₂CONH₂ | CH₃ | CH₂CH═CH₂ |
| B-487 | H | CH₂CH₂CONH₂ | CH₃ | CH₂CH═CH₂ |
| B-488 | H | H | H | CH₂Ph |
| B-489 | H | CH₃ | H | CH₂Ph |
| B-490 | H | CH₃CH₂ | H | CH₂Ph |
| B-491 | H | n-C₃H₇ | H | CH₂Ph |
| B-492 | H | i-C₃H₇ | H | CH₂Ph |
| B-493 | H | n-C₄H₉ | H | CH₂Ph |
| B-494 | H | i-C₄H₉ | H | CH₂Ph |
| B-495 | H | s-C₄H₉ | H | CH₂Ph |
| B-496 | H | t-C₄H₉ | H | CH₂Ph |
| B-497 | H | CH₂Ph | H | CH₂Ph |
| B-498 | H | Ph | H | CH₂Ph |
| B-499 | H | CH₂OH | H | CH₂Ph |
| B-500 | H | CH(OH)CH₃ | H | CH₂Ph |
| B-501 | H | CH₂SH | H | CH₂Ph |
| B-502 | H | CH₂SCH₂CH₃ | H | CH₂Ph |
| B-503 | H | CH₂CONH₂ | H | CH₂Ph |
| B-504 | H | CH₂CH₂CONH₂ | H | CH₂Ph |
| B-505 | H | CH₃ | CH₃ | CH₂Ph |
| B-506 | H | CH₃CH₂ | CH₃ | CH₂Ph |
| B-507 | H | n-C₃H₇ | CH₃ | CH₂Ph |
| B-508 | H | i-C₃H₇ | CH₃ | CH₂Ph |
| B-509 | H | n-C₄H₉ | CH₃ | CH₂Ph |
| B-510 | H | i-C₄H₉ | CH₃ | CH₂Ph |
| B-511 | H | s-C₄H₉ | CH₃ | CH₂Ph |
| B-512 | H | t-C₄H₉ | CH₃ | CH₂Ph |
| B-513 | H | CH₂Ph | CH₃ | CH₂Ph |
| B-514 | H | Ph | CH₃ | CH₂Ph |
| B-515 | H | CH₂OH | CH₃ | CH₂Ph |
| B-516 | H | CH(OH)CH₃ | CH₃ | CH₂Ph |
| B-517 | H | CH₂SH | CH₃ | CH₂Ph |
| B-518 | H | CH₂SCH₂CH₃ | CH₃ | CH₂Ph |
| B-519 | H | CH₂CONH₂ | CH₃ | CH₂Ph |
| B-520 | H | CH₂CH₂CONH₂ | CH₃ | CH₂Ph |
| B-521 | H | H | H | CH₂(Ph-2-Cl) |
| B-522 | H | H | H | CH₂(Ph-3-Cl) |
| B-523 | H | H | H | CH₂(Ph-4-Cl) |
| B-524 | H | CH₃ | H | CH₂(Ph-2-Cl) |
| B-525 | H | CH₃ | H | CH₂(Ph-3-Cl) |
| B-526 | H | CH₃ | H | CH₂(Ph-4-Cl) |
| B-527 | H | CH₃ | CH₃ | CH₂(Ph-2-Cl) |
| B-528 | H | CH₃ | CH₃ | CH₂(Ph-3-Cl) |
| B-529 | H | CH₃ | CH₃ | CH₂(Ph-4-Cl) |
| B-530 | H | H | H | Ph |
| B-531 | H | CH₃ | H | Ph |
| B-532 | H | CH₃ | CH₃ | Ph |
| B-533 | H | H | H | 2-CH₃-4-C₃F₇—Ph |
| B-534 | H | CH₃ | H | 2-CH₃-4-C₃F₇—Ph |
| B-535 | H | CH₃ | CH₃ | 2-CH₃-4-C₃F₇—Ph |
| B-536 | H | H | H | CH₂CO₂CH₃ |
| B-537 | H | CH₃ | H | CH₂CO₂CH₃ |
| B-538 | H | CH₃ | CH₃ | CH₂CO₂CH₃ |
| B-539 | H | H | H | C(CH₃)₂CO₂CH₂Ph |
| B-540 | H | CH₃ | H | C(CH₃)₂CO₂CH₂Ph |
| B-541 | H | CH₃ | CH₃ | C(CH₃)₂CO₂CH₂Ph |

TABLE 3
Compounds of formula (Ic):

| | |
|---|---|
| | (Ic) |
| 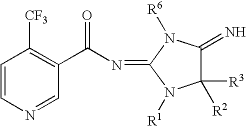 | |

| Com- | | | | |
|---|---|---|---|---|
| pound | R¹ | R² | R³ | R⁶ |
| C-1 | H | H | H | H |
| C-2 | CH₃ | H | H | H |
| C-3 | CH₂CH═CH₂ | H | H | H |
| C-4 | CH₂Ph | H | H | H |
| C-5 | Ph | H | H | H |
| C-6 | H | CH₃ | H | H |
| C-7 | CH₃ | CH₃ | H | H |
| C-8 | CH₂CH═CH₂ | CH₃ | H | H |
| C-9 | CH₂Ph | CH₃ | H | H |
| C-10 | Ph | CH₃ | H | H |
| C-11 | H | CH₃CH₂ | H | H |
| C-12 | CH₃ | CH₃CH₂ | H | H |
| C-13 | CH₂CH═CH₂ | CH₃CH₂ | H | H |
| C-14 | CH₂Ph | CH₃CH₂ | H | H |
| C-15 | Ph | CH₃CH₂ | H | H |
| C-16 | H | n-C₃H₇ | H | H |
| C-17 | CH₃ | n-C₃H₇ | H | H |
| C-18 | CH₂CH═CH₂ | n-C₃H₇ | H | H |
| C-19 | CH₂Ph | n-C₃H₇ | H | H |
| C-20 | Ph | n-C₃H₇ | H | H |
| C-21 | H | i-C₃H₇ | H | H |
| C-22 | CH₃ | i-C₃H₇ | H | H |
| C-23 | CH₂CH═CH₂ | i-C₃H₇ | H | H |
| C-24 | CH₂Ph | i-C₃H₇ | H | H |
| C-25 | Ph | i-C₃H₇ | H | H |
| C-26 | H | n-C₄H₉ | H | H |
| C-27 | CH₃ | n-C₄H₉ | H | H |
| C-28 | CH₂CH═CH₂ | n-C₄H₉ | H | H |
| C-29 | CH₂Ph | n-C₄H₉ | H | H |
| C-30 | Ph | n-C₄H₉ | H | H |
| C-31 | H | i-C₄H₉ | H | H |
| C-32 | CH₃ | i-C₄H₉ | H | H |
| C-33 | CH₂CH═CH₂ | i-C₄H₉ | H | H |
| C-34 | CH₂Ph | i-C₄H₉ | H | H |
| C-35 | Ph | i-C₄H₉ | H | H |
| C-36 | H | s-C₄H₉ | H | H |
| C-37 | CH₃ | s-C₄H₉ | H | H |
| C-38 | CH₂CH═CH₂ | s-C₄H₉ | H | H |
| C-39 | CH₂Ph | s-C₄H₉ | H | H |
| C-40 | Ph | s-C₄H₉ | H | H |
| C-41 | H | t-C₄H₉ | H | H |
| C-42 | CH₃ | t-C₄H₉ | H | H |
| C-43 | CH₂CH═CH₂ | t-C₄H₉ | H | H |
| C-44 | CH₂Ph | t-C₄H₉ | H | H |
| C-45 | Ph | t-C₄H₉ | H | H |
| C-46 | H | CH₂Ph | H | H |
| C-47 | CH₃ | CH₂Ph | H | H |
| C-48 | CH₂CH═CH₂ | CH₂Ph | H | H |
| C-49 | CH₂Ph | CH₂Ph | H | H |
| C-50 | Ph | CH₂Ph | H | H |
| C-51 | H | Ph | H | H |
| C-52 | CH₃ | Ph | H | H |
| C-53 | CH₂CH═CH₂ | Ph | H | H |
| C-54 | CH₂Ph | Ph | H | H |
| C-55 | Ph | Ph | H | H |
| C-56 | H | CH₂OH | H | H |
| C-57 | CH₃ | CH₂OH | H | H |
| C-58 | CH₂CH═CH₂ | CH₂OH | H | H |
| C-59 | CH₂Ph | CH₂OH | H | H |
| C-60 | Ph | CH₂OH | H | H |
| C-61 | H | CH(OH)CH₃ | H | H |
| C-62 | CH₃ | CH(OH)CH₃ | H | H |
| C-63 | CH₂CH═CH₂ | CH(OH)CH₃ | H | H |
| C-64 | CH₂Ph | CH(OH)CH₃ | H | H |
| C-65 | Ph | CH(OH)CH₃ | H | H |
| C-66 | H | CH₂SH | H | H |
| C-67 | CH₃ | CH₂SH | H | H |
| C-68 | CH₂CH═CH₂ | CH₂SH | H | H |
| C-69 | CH₂Ph | CH₂SH | H | H |
| C-70 | Ph | CH₂SH | H | H |
| C-71 | H | CH₂SCH₂CH₃ | H | H |
| C-72 | CH₃ | CH₂SCH₂CH₃ | H | H |
| C-73 | CH₂CH═CH₂ | CH₂SCH₂CH₃ | H | H |
| C-74 | CH₂Ph | CH₂SCH₂CH₃ | H | H |
| C-75 | Ph | CH₂SCH₂CH₃ | H | H |
| C-76 | H | CH₂CONH₂ | H | H |
| C-77 | CH₃ | CH₂CONH₂ | H | H |
| C-78 | CH₂CH═CH₂ | CH₂CONH₂ | H | H |
| C-79 | CH₂Ph | CH₂CONH₂ | H | H |
| C-80 | Ph | CH₂CONH₂ | H | H |
| C-81 | H | CH₂CH₂CONH₂ | H | H |
| C-82 | CH₃ | CH₂CH₂CONH₂ | H | H |
| C-83 | CH₂CH═CH₂ | CH₂CH₂CONH₂ | H | H |
| C-84 | CH₂Ph | CH₂CH₂CONH₂ | H | H |
| C-85 | Ph | CH₂CH₂CONH₂ | H | H |
| C-86 | H | CH₃ | CH₃ | H |
| C-87 | CH₃ | CH₃ | CH₃ | H |
| C-88 | CH₂CH═CH₂ | CH₃ | CH₃ | H |
| C-89 | CH₂Ph | CH₃ | CH₃ | H |
| C-90 | Ph | CH₃ | CH₃ | H |
| C-91 | H | CH₃CH₂ | CH₃ | H |
| C-92 | CH₃ | CH₃CH₂ | CH₃ | H |
| C-93 | CH₂CH═CH₂ | CH₃CH₂ | CH₃ | H |
| C-94 | CH₂Ph | CH₃CH₂ | CH₃ | H |
| C-95 | Ph | CH₃CH₂ | CH₃ | H |
| C-96 | H | n-C₃H₇ | CH₃ | H |
| C-97 | CH₃ | n-C₃H₇ | CH₃ | H |
| C-98 | CH₂CH═CH₂ | n-C₃H₇ | CH₃ | H |
| C-99 | CH₂Ph | n-C₃H₇ | CH₃ | H |
| C-100 | Ph | n-C₃H₇ | CH₃ | H |
| C-101 | H | i-C₃H₇ | CH₃ | H |
| C-102 | CH₃ | i-C₃H₇ | CH₃ | H |
| C-103 | CH₂CH═CH₂ | i-C₃H₇ | CH₃ | H |
| C-104 | CH₂Ph | i-C₃H₇ | CH₃ | H |
| C-105 | Ph | i-C₃H₇ | CH₃ | H |
| C-106 | H | n-C₄H₉ | CH₃ | H |
| C-107 | CH₃ | n-C₄H₉ | CH₃ | H |
| C-108 | CH₂CH═CH₂ | n-C₄H₉ | CH₃ | H |
| C-109 | CH₂Ph | n-C₄H₉ | CH₃ | H |
| C-110 | Ph | n-C₄H₉ | CH₃ | H |
| C-111 | H | i-C₄H₉ | CH₃ | H |
| C-112 | CH₃ | i-C₄H₉ | CH₃ | H |
| C-113 | CH₂CH═CH₂ | i-C₄H₉ | CH₃ | H |
| C-114 | CH₂Ph | i-C₄H₉ | CH₃ | H |
| C-115 | Ph | i-C₄H₉ | CH₃ | H |
| C-116 | H | s-C₄H₉ | CH₃ | H |
| C-117 | CH₃ | s-C₄H₉ | CH₃ | H |
| C-118 | CH₂CH═CH₂ | s-C₄H₉ | CH₃ | H |
| C-119 | CH₂Ph | s-C₄H₉ | CH₃ | H |
| C-120 | Ph | s-C₄H₉ | CH₃ | H |
| C-121 | H | t-C₄H₉ | CH₃ | H |
| C-122 | CH₃ | t-C₄H₉ | CH₃ | H |
| C-123 | CH₂CH═CH₂ | t-C₄H₉ | CH₃ | H |
| C-124 | CH₂Ph | t-C₄H₉ | CH₃ | H |
| C-125 | Ph | t-C₄H₉ | CH₃ | H |
| C-126 | H | CH₂Ph | CH₃ | H |
| C-127 | CH₃ | CH₂Ph | CH₃ | H |
| C-128 | CH₂CH═CH₂ | CH₂Ph | CH₃ | H |
| C-129 | CH₂Ph | CH₂Ph | CH₃ | H |
| C-130 | Ph | CH₂Ph | CH₃ | H |
| C-131 | H | Ph | CH₃ | H |
| C-132 | CH₃ | Ph | CH₃ | H |
| C-133 | CH₂CH═CH₂ | Ph | CH₃ | H |
| C-134 | CH₂Ph | Ph | CH₃ | H |
| C-135 | Ph | Ph | CH₃ | H |
| C-136 | H | C₂H₅ | C₂H₅ | H |
| C-137 | CH₃ | C₂H₅ | C₂H₅ | H |
| C-138 | CH₂CH═CH₂ | C₂H₅ | C₂H₅ | H |
| C-139 | CH₂Ph | C₂H₅ | C₂H₅ | H |
| C-140 | Ph | C₂H₅ | C₂H₅ | H |
| C-141 | H | n-C₃H₇ | C₂H₅ | H |
| C-142 | CH₃ | n-C₃H₇ | C₂H₅ | H |
| C-143 | CH₂CH═CH₂ | n-C₃H₇ | C₂H₅ | H |
| C-144 | CH₂Ph | n-C₃H₇ | C₂H₅ | H |
| C-145 | Ph | n-C₃H₇ | C₂H₅ | H |
| C-146 | H | i-C₃H₇ | C₂H₅ | H |
| C-147 | CH₃ | i-C₃H₇ | C₂H₅ | H |
| C-148 | CH₂CH═CH₂ | i-C₃H₇ | C₂H₅ | H |
| C-149 | CH₂Ph | i-C₃H₇ | C₂H₅ | H |
| C-150 | Ph | i-C₃H₇ | C₂H₅ | H |
| C-151 | H | n-C₄H₉ | C₂H₅ | H |
| C-152 | CH₃ | n-C₄H₉ | C₂H₅ | H |
| C-153 | CH₂CH═CH₂ | n-C₄H₉ | C₂H₅ | H |
| C-154 | CH₂Ph | n-C₄H₉ | C₂H₅ | H |
| C-155 | Ph | n-C₄H₉ | C₂H₅ | H |
| C-156 | H | i-C₄H₉ | C₂H₅ | H |
| C-157 | CH₃ | i-C₄H₉ | C₂H₅ | H |
| C-158 | CH₂CH═CH₂ | i-C₄H₉ | C₂H₅ | H |
| C-159 | CH₂Ph | i-C₄H₉ | C₂H₅ | H |
| C-160 | Ph | i-C₄H₉ | C₂H₅ | H |
| C-161 | H | s-C₄H₉ | C₂H₅ | H |
| C-162 | CH₃ | s-C₄H₉ | C₂H₅ | H |
| C-163 | CH₂CH═CH₂ | s-C₄H₉ | C₂H₅ | H |
| C-164 | CH₂Ph | s-C₄H₉ | C₂H₅ | H |
| C-165 | Ph | s-C₄H₉ | C₂H₅ | H |
| C-166 | H | t-C₄H₉ | C₂H₅ | H |
| C-167 | CH₃ | t-C₄H₉ | C₂H₅ | H |
| C-168 | CH₂CH═CH₂ | t-C₄H₉ | C₂H₅ | H |
| C-169 | CH₂Ph | t-C₄H₉ | C₂H₅ | H |
| C-170 | Ph | t-C₄H₉ | C₂H₅ | H |
| C-171 | H | CH₂Ph | C₂H₅ | H |
| C-172 | CH₃ | CH₂Ph | C₂H₅ | H |
| C-173 | CH₂CH═CH₂ | CH₂Ph | C₂H₅ | H |
| C-174 | CH₂Ph | CH₂Ph | C₂H₅ | H |
| C-175 | Ph | CH₂Ph | C₂H₅ | H |
| C-176 | H | Ph | C₂H₅ | H |
| C-177 | CH₃ | Ph | C₂H₅ | H |
| C-178 | CH₂CH═CH₂ | Ph | C₂H₅ | H |
| C-179 | CH₂Ph | Ph | C₂H₅ | H |
| C-180 | Ph | Ph | C₂H₅ | H |
| C-181 | H | CH₂CH₂ | | H |
| C-182 | CH₃ | CH₂CH₂ | | H |
| C-183 | CH₂CH═CH₂ | CH₂CH₂ | | H |
| C-184 | CH₂Ph | CH₂CH₂ | | H |
| C-185 | Ph | CH₂CH₂ | | H |
| C-186 | H | CH₂H₂CH₂ | | H |
| C-187 | CH₃ | CH₂H₂CH₂ | | H |
| C-188 | CH₂CH═CH₂ | CH₂H₂CH₂ | | H |
| C-189 | CH₂Ph | CH₂H₂CH₂ | | H |
| C-190 | Ph | HC₂H₂CH₂ | | H |
| C-191 | H | CH₂CH₂CH₂CH₂ | | H |
| C-192 | CH₃ | CH₂CH₂CH₂CH₂ | | H |
| C-193 | CH₂CH═CH₂ | CH₂CH₂CH₂CH₂ | | H |
| C-194 | CH₂Ph | CH₂CH₂CH₂CH₂ | | H |
| C-195 | Ph | CH₂CH₂CH₂CH₂ | | H |
| C-196 | H | CH₂CH₂CH₂CH₂CH₂ | | H |
| C-197 | CH₃ | CH₂CH₂CH₂CH₂CH₂ | | H |
| C-198 | CH₂CH═CH₂ | CH₂CH₂CH₂CH₂CH₂ | | H |
| C-199 | CH₂Ph | CH₂CH₂CH₂CH₂CH₂ | | H |
| C-200 | Ph | CH₂CH₂CH₂CH₂CH₂ | | H |
| C-201 | H | H | H | CH₃ |
| C-202 | CH₃ | H | H | CH₃ |
| C-203 | CH₂CH═CH₂ | H | H | CH₃ |
| C-204 | CH₂Ph | H | H | CH₃ |
| C-205 | Ph | H | H | CH₃ |
| C-206 | H | CH₃ | H | CH₃ |
| C-207 | CH₃ | CH₃ | H | CH₃ |
| C-208 | CH₂CH═CH₂ | CH₃ | H | CH₃ |
| C-209 | CH₂Ph | CH₃ | H | CH₃ |
| C-210 | Ph | CH₃ | H | CH₃ |
| C-211 | H | CH₃CH₂ | H | CH₃ |
| C-212 | CH₃ | CH₃CH₂ | H | CH₃ |
| C-213 | CH₂CH═CH₂ | CH₃CH₂ | H | CH₃ |
| C-214 | CH₂Ph | CH₃CH₂ | H | CH₃ |
| C-215 | Ph | CH₃CH₂ | H | CH₃ |
| C-216 | H | n-C₃H₇ | H | CH₃ |
| C-217 | CH₃ | n-C₃H₇ | H | CH₃ |
| C-218 | CH₂CH═CH₂ | n-C₃H₇ | H | CH₃ |
| C-219 | CH₂Ph | n-C₃H₇ | H | CH₃ |
| C-220 | Ph | n-C₃H₇ | H | CH₃ |
| C-221 | H | i-C₃H₇ | H | CH₃ |
| C-222 | CH₃ | i-C₃H₇ | H | CH₃ |
| C-223 | CH₂CH═CH₂ | i-C₃H₇ | H | CH₃ |
| C-224 | CH₂Ph | i-C₃H₇ | H | CH₃ |
| C-225 | Ph | i-C₃H₇ | H | CH₃ |
| C-226 | H | n-C₄H₉ | H | CH₃ |
| C-227 | CH₃ | n-C₄H₉ | H | CH₃ |
| C-228 | CH₂CH═CH₂ | n-C₄H₉ | H | CH₃ |
| C-229 | CH₂Ph | n-C₄H₉ | H | CH₃ |
| C-230 | Ph | n-C₄H₉ | H | CH₃ |
| C-231 | H | i-C₄H₉ | H | CH₃ |
| C-232 | CH₃ | i-C₄H₉ | H | CH₃ |
| C-233 | CH₂CH═CH₂ | i-C₄H₉ | H | CH₃ |
| C-234 | CH₂Ph | i-C₄H₉ | H | CH₃ |
| C-235 | Ph | i-C₄H₉ | H | CH₃ |
| C-236 | H | s-C₄H₉ | H | CH₃ |
| C-237 | CH₃ | s-C₄H₉ | H | CH₃ |
| C-238 | CH₂CH═CH₂ | s-C₄H₉ | H | CH₃ |
| C-239 | CH₂Ph | s-C₄H₉ | H | CH₃ |
| C-240 | Ph | s-C₄H₉ | H | CH₃ |
| C-241 | H | t-C₄H₉ | H | CH₃ |
| C-242 | CH₃ | t-C₄H₉ | H | CH₃ |
| C-243 | CH₂CH═CH₂ | t-C₄H₉ | H | CH₃ |
| C-244 | CH₂Ph | t-C₄H₉ | H | CH₃ |
| C-245 | Ph | t-C₄H₉ | H | CH₃ |
| C-246 | H | CH₂Ph | H | CH₃ |
| C-247 | CH₃ | CH₂Ph | H | CH₃ |
| C-248 | CH₂CH═CH₂ | CH₂Ph | H | CH₃ |
| C-249 | CH₂Ph | CH₂Ph | H | CH₃ |
| C-250 | Ph | CH₂Ph | H | CH₃ |
| C-251 | H | Ph | H | CH₃ |
| C-252 | CH₃ | Ph | H | CH₃ |
| C-253 | CH₂CH═CH₂ | Ph | H | CH₃ |
| C-254 | CH₂Ph | Ph | H | CH₃ |
| C-255 | Ph | Ph | H | CH₃ |
| C-256 | H | CH₂OH | H | CH₃ |
| C-257 | CH₃ | CH₂OH | H | CH₃ |
| C-258 | CH₂CH═CH₂ | CH₂OH | H | CH₃ |
| C-259 | CH₂Ph | CH₂OH | H | CH₃ |
| C-260 | Ph | CH₂OH | H | CH₃ |
| C-261 | H | CH(OH)CH₃ | H | CH₃ |
| C-262 | CH₃ | CH(OH)CH₃ | H | CH₃ |
| C-263 | CH₂CH═CH₂ | CH(OH)CH₃ | H | CH₃ |
| C-264 | CH₂Ph | CH(OH)CH₃ | H | CH₃ |
| C-265 | Ph | CH(OH)CH₃ | H | CH₃ |
| C-266 | H | CH₂SH | H | CH₃ |
| C-267 | CH₃ | CH₂SH | H | CH₃ |
| C-268 | CH₂CH═CH₂ | CH₂SH | H | CH₃ |
| C-269 | CH₂Ph | CH₂SH | H | CH₃ |
| C-270 | Ph | CH₂SH | H | CH₃ |
| C-271 | H | CH₂SCH₂CH₃ | H | CH₃ |
| C-272 | CH₃ | CH₂SCH₂CH₃ | H | CH₃ |
| C-273 | CH₂CH═CH₂ | CH₂SCH₂CH₃ | H | CH₃ |
| C-274 | CH₂Ph | CH₂SCH₂CH₃ | H | CH₃ |
| C-275 | Ph | CH₂SCH₂CH₃ | H | CH₃ |
| C-276 | H | CH₂CONH₂ | H | CH₃ |
| C-277 | CH₃ | CH₂CONH₂ | H | CH₃ |
| C-278 | CH₂CH═CH₂ | CH₂CONH₂ | H | CH₃ |
| C-279 | CH₂Ph | CH₂CONH₂ | H | CH₃ |
| C-280 | Ph | CH₂CONH₂ | H | CH₃ |
| C-281 | H | CH₂CH₂CONH₂ | H | CH₃ |
| C-282 | CH₃ | CH₂CH₂CONH₂ | H | CH₃ |
| C-283 | CH₂CH═CH₂ | CH₂CH₂CONH₂ | H | CH₃ |
| C-284 | CH₂Ph | CH₂CH₂CONH₂ | H | CH₃ |
| C-285 | Ph | CH₂CH₂CONH₂ | H | CH₃ |
| C-286 | H | CH₃ | CH₃ | CH₃ |
| C-287 | CH₃ | CH₃ | CH₃ | CH₃ |
| C-288 | CH₂CH═CH₂ | CH₃ | CH₃ | CH₃ |
| C-289 | CH₂Ph | CH₃ | CH₃ | CH₃ |
| C-290 | Ph | CH₃ | CH₃ | CH₃ |
| C-291 | H | CH₃CH₂ | CH₃ | CH₃ |
| C-292 | CH₃ | CH₃CH₂ | CH₃ | CH₃ |
| C-293 | CH₂CH═CH₂ | CH₃CH₂ | CH₃ | CH₃ |
| C-294 | CH₂Ph | CH₃CH₂ | CH₃ | CH₃ |
| C-295 | Ph | CH₃CH₂ | CH₃ | CH₃ |
| C-296 | H | n-C₃H₇ | CH₃ | CH₃ |
| C-297 | CH₃ | n-C₃H₇ | CH₃ | CH₃ |
| C-298 | CH₂CH═CH₂ | n-C₃H₇ | CH₃ | CH₃ |
| C-299 | CH₂Ph | n-C₃H₇ | CH₃ | CH₃ |
| C-300 | Ph | n-C₃H₇ | CH₃ | CH₃ |
| C-301 | H | i-C₃H₇ | CH₃ | CH₃ |
| C-302 | CH₃ | i-C₃H₇ | CH₃ | CH₃ |
| C-303 | CH₂CH═CH₂ | i-C₃H₇ | CH₃ | CH₃ |
| C-304 | CH₂Ph | i-C₃H₇ | CH₃ | CH₃ |
| C-305 | Ph | i-C₃H₇ | CH₃ | CH₃ |
| C-306 | H | n-C₄H₉ | CH₃ | CH₃ |
| C-307 | CH₃ | n-C₄H₉ | CH₃ | CH₃ |
| C-308 | CH₂CH═CH₂ | n-C₄H₉ | CH₃ | CH₃ |
| C-309 | CH₂Ph | n-C₄H₉ | CH₃ | CH₃ |
| C-310 | Ph | n-C₄H₉ | CH₃ | CH₃ |
| C-311 | H | i-C₄H₉ | CH₃ | CH₃ |
| C-312 | CH₃ | i-C₄H₉ | CH₃ | CH₃ |
| C-313 | CH₂CH═CH₂ | i-C₄H₉ | CH₃ | CH₃ |
| C-314 | CH₂Ph | i-C₄H₉ | CH₃ | CH₃ |
| C-315 | Ph | i-C₄H₉ | CH₃ | CH₃ |
| C-316 | H | s-C₄H₉ | CH₃ | CH₃ |
| C-317 | CH₃ | s-C₄H₉ | CH₃ | CH₃ |
| C-318 | CH₂CH═CH₂ | s-C₄H₉ | CH₃ | CH₃ |
| C-319 | CH₂Ph | s-C₄H₉ | CH₃ | CH₃ |
| C-320 | Ph | s-C₄H₉ | CH₃ | CH₃ |
| C-321 | H | t-C₄H₉ | CH₃ | CH₃ |
| C-322 | CH₃ | t-C₄H₉ | CH₃ | CH₃ |
| C-323 | CH₂CH═CH₂ | t-C₄H₉ | CH₃ | CH₃ |
| C-324 | CH₂Ph | t-C₄H₉ | CH₃ | CH₃ |
| C-325 | Ph | t-C₄H₉ | CH₃ | CH₃ |
| C-326 | H | CH₂Ph | CH₃ | CH₃ |
| C-327 | CH₃ | CH₂Ph | CH₃ | CH₃ |
| C-328 | CH₂CH═CH₂ | CH₂Ph | CH₃ | CH₃ |
| C-329 | CH₂Ph | CH₂Ph | CH₃ | CH₃ |
| C-330 | Ph | CH₂Ph | CH₃ | CH₃ |
| C-331 | H | Ph | CH₃ | CH₃ |
| C-332 | CH₃ | Ph | CH₃ | CH₃ |
| C-333 | CH₂CH═CH₂ | Ph | CH₃ | CH₃ |
| C-334 | CH₂Ph | Ph | CH₃ | CH₃ |
| C-335 | Ph | Ph | CH₃ | CH₃ |
| C-336 | H | CH₂CH₂ | | CH₃ |
| C-337 | CH₃ | CH₂CH₂ | | CH₃ |
| C-338 | CH₂CH═CH₂ | CH₂CH₂ | | CH₃ |
| C-339 | CH₂Ph | CH₂CH₂ | | CH₃ |
| C-340 | Ph | CH₂CH₂ | | CH₃ |
| C-341 | H | CH₂H₂CH₂ | | CH₃ |
| C-342 | CH₃ | CH₂H₂CH₂ | | CH₃ |
| C-343 | CH₂CH═CH₂ | CH₂H₂CH₂ | | CH₃ |
| C-344 | CH₂Ph | CH₂H₂CH₂ | | CH₃ |
| C-345 | Ph | CH₂H₂CH₂ | | CH₃ |
| C-346 | H | CH₂CH₂CH₂CH₂ | | CH₃ |
| C-347 | CH₃ | CH₂CH₂CH₂CH₂ | | CH₃ |
| C-348 | CH₂CH═CH₂ | CH₂CH₂CH₂CH₂ | | CH₃ |
| C-349 | CH₂Ph | CH₂CH₂CH₂CH₂ | | CH₃ |
| C-350 | Ph | CH₂CH₂CH₂CH₂ | | CH₃ |
| C-351 | H | CH₂CH₂CH₂CH₂CH₂ | | CH₃ |
| C-352 | CH₃ | CH₂CH₂CH₂CH₂CH₂ | | CH₃ |
| C-353 | CH₂CH═CH₂ | CH₂CH₂CH₂CH₂CH₂ | | CH₃ |
| C-354 | CH₂Ph | CH₂CH₂CH₂CH₂CH₂ | | CH₃ |
| C-355 | Ph | CH₂CH₂CH₂CH₂CH₂ | | CH₃ |
| C-356 | H | H | H | CH₃CH₂ |
| C-357 | H | CH₃ | H | CH₃CH₂ |
| C-358 | H | CH₃CH₂ | H | CH₃CH₂ |
| C-359 | H | n-C₃H₇ | H | CH₃CH₂ |
| C-360 | H | i-C₃H₇ | H | CH₃CH₂ |
| C-361 | H | n-C₄H₉ | H | CH₃CH₂ |
| C-362 | H | i-C₄H₉ | H | CH₃CH₂ |
| C-363 | H | s-C₄H₉ | H | CH₃CH₂ |
| C-364 | H | t-C₄H₉ | H | CH₃CH₂ |
| C-365 | H | CH₂Ph | H | CH₃CH₂ |
| C-366 | H | Ph | H | CH₃CH₂ |
| C-367 | H | CH₂OH | H | CH₃CH₂ |
| C-368 | H | CH(OH)CH₃ | H | CH₃CH₂ |
| C-369 | H | CH₂SH | H | CH₃CH₂ |
| C-370 | H | CH₂SCH₂CH₃ | H | CH₃CH₂ |
| C-371 | H | CH₂CONH₂ | H | CH₃CH₂ |
| C-372 | H | CH₂CH₂CONH₂ | H | CH₃CH₂ |
| C-373 | H | CH₃ | CH₃ | CH₃CH₂ |
| C-374 | H | CH₃CH₂ | CH₃ | CH₃CH₂ |
| C-375 | H | n-C₃H₇ | CH₃ | CH₃CH₂ |
| C-376 | H | i-C₃H₇ | CH₃ | CH₃CH₂ |
| C-377 | H | n-C₄H₉ | CH₃ | CH₃CH₂ |
| C-378 | H | i-C₄H₉ | CH₃ | CH₃CH₂ |
| C-379 | H | s-C₄H₉ | CH₃ | CH₃CH₂ |
| C-380 | H | t-C₄H₉ | CH₃ | CH₃CH₂ |
| C-381 | H | CH₂Ph | CH₃ | CH₃CH₂ |
| C-382 | H | Ph | CH₃ | CH₃CH₂ |
| C-383 | H | CH₂OH | CH₃ | CH₃CH₂ |
| C-384 | H | CH(OH)CH₃ | CH₃ | CH₃CH₂ |
| C-385 | H | CH₂SH | CH₃ | CH₃CH₂ |
| C-386 | H | CH₂SCH₂CH₃ | CH₃ | CH₃CH₂ |
| C-387 | H | CH₂CONH₂ | CH₃ | CH₃CH₂ |
| C-388 | H | CH₂CH₂CONH₂ | CH₃ | CH₃CH₂ |
| C-389 | H | H | H | i-C₃H₇ |
| C-390 | H | CH₃ | H | i-C₃H₇ |
| C-391 | H | CH₃CH₂ | H | i-C₃H₇ |
| C-392 | H | n-C₃H₇ | H | i-C₃H₇ |
| C-393 | H | i-C₃H₇ | H | i-C₃H₇ |
| C-394 | H | n-C₄H₉ | H | i-C₃H₇ |
| C-395 | H | i-C₄H₉ | H | i-C₃H₇ |
| C-396 | H | s-C₄H₉ | H | i-C₃H₇ |
| C-397 | H | t-C₄H₉ | H | i-C₃H₇ |
| C-398 | H | CH₂Ph | H | i-C₃H₇ |
| C-399 | H | Ph | H | i-C₃H₇ |
| C-400 | H | CH₂OH | H | i-C₃H₇ |
| C-401 | H | CH(OH)CH₃ | H | i-C₃H₇ |
| C-402 | H | CH₂SH | H | i-C₃H₇ |
| C-403 | H | CH₂SCH₂CH₃ | H | i-C₃H₇ |
| C-404 | H | CH₂CONH₂ | H | i-C₃H₇ |
| C-405 | H | CH₂CH₂CONH₂ | H | i-C₃H₇ |
| C-406 | H | CH₃ | H | i-C₃H₇ |
| C-407 | H | CH₃CH₂ | CH₃ | i-C₃H₇ |
| C-408 | H | n-C₃H₇ | CH₃ | i-C₃H₇ |
| C-409 | H | i-C₃H₇ | CH₃ | i-C₃H₇ |
| C-410 | H | n-C₄H₉ | CH₃ | i-C₃H₇ |
| C-411 | H | i-C₄H₉ | CH₃ | i-C₃H₇ |
| C-412 | H | s-C₄H₉ | CH₃ | i-C₃H₇ |
| C-413 | H | t-C₄H₉ | CH₃ | i-C₃H₇ |
| C-414 | H | CH₂Ph | CH₃ | i-C₃H₇ |
| C-415 | H | Ph | CH₃ | i-C₃H₇ |
| C-416 | H | CH₂OH | CH₃ | i-C₃H₇ |
| C-417 | H | CH(OH)CH₃ | CH₃ | i-C₃H₇ |
| C-418 | H | CH₂SH | CH₃ | i-C₃H₇ |
| C-419 | H | CH₂SCH₂CH₃ | CH₃ | i-C₃H₇ |
| C-420 | H | CH₂CONH₂ | CH₃ | i-C₃H₇ |
| C-421 | H | CH₂CH₂CONH₂ | CH₃ | i-C₃H₇ |
| C-422 | H | H | H | t-C₄H₉ |
| C-423 | H | CH₃ | H | t-C₄H₉ |
| C-424 | H | CH₃CH₂ | H | t-C₄H₉ |
| C-425 | H | n-C₃H₇ | H | t-C₄H₉ |
| C-426 | H | i-C₃H₇ | H | t-C₄H₉ |
| C-427 | H | n-C₄H₉ | H | t-C₄H₉ |
| C-428 | H | i-C₄H₉ | H | t-C₄H₉ |
| C-429 | H | s-C₄H₉ | H | t-C₄H₉ |
| C-430 | H | t-C₄H₉ | H | t-C₄H₉ |
| C-431 | H | CH₂Ph | H | t-C₄H₉ |
| C-432 | H | Ph | H | t-C₄H₉ |
| C-433 | H | CH₂OH | H | t-C₄H₉ |
| C-434 | H | CH(OH)CH₃ | H | t-C₄H₉ |
| C-435 | H | CH₂SH | H | t-C₄H₉ |
| C-436 | H | CH₂SCH₂CH₃ | H | t-C₄H₉ |
| C-437 | H | CH₂CONH₂ | H | t-C₄H₉ |
| C-438 | H | CH₂CH₂CONH₂ | H | t-C₄H₉ |
| C-439 | H | CH₃ | CH₃ | t-C₄H₉ |
| C-440 | H | CH₃CH₂ | CH₃ | t-C₄H₉ |
| C-441 | H | n-C₃H₇ | CH₃ | t-C₄H₉ |
| C-442 | H | i-C₃H₇ | CH₃ | t-C₄H₉ |
| C-443 | H | n-C₄H₉ | CH₃ | t-C₄H₉ |
| C-444 | H | i-C₄H₉ | CH₃ | t-C₄H₉ |
| C-445 | H | s-C₄H₉ | CH₃ | t-C₄H₉ |
| C-446 | H | t-C₄H₉ | CH₃ | t-C₄H₉ |
| C-447 | H | CH₂Ph | CH₃ | t-C₄H₉ |
| C-448 | H | Ph | CH₃ | t-C₄H₉ |
| C-449 | H | CH₂OH | CH₃ | t-C₄H₉ |
| C-450 | H | CH(OH)CH₃ | CH₃ | t-C₄H₉ |
| C-451 | H | CH₂SH | CH₃ | t-C₄H₉ |
| C-452 | H | CH₂SCH₂CH₃ | CH₃ | t-C₄H₉ |
| C-453 | H | CH₂CONH₂ | CH₃ | t-C₄H₉ |
| C-454 | H | CH₂CH₂CONH₂ | CH₃ | t-C₄H₉ |
| C-455 | H | H | H | CH₂CH═CH₂ |
| C-456 | H | CH₃ | H | CH₂CH═CH₂ |
| C-457 | H | CH₃CH₂ | H | CH₂CH═CH₂ |
| C-458 | H | n-C₃H₇ | H | CH₂CH═CH₂ |
| C-459 | H | i-C₃H₇ | H | CH₂CH═CH₂ |
| C-460 | H | n-C₄H₉ | H | CH₂CH═CH₂ |
| C-461 | H | i-C₄H₉ | H | CH₂CH═CH₂ |
| C-462 | H | s-C₄H₉ | H | CH₂CH═CH₂ |
| C-463 | H | t-C₄H₉ | H | CH₂CH═CH₂ |
| C-464 | H | CH₂Ph | H | CH₂CH═CH₂ |
| C-465 | H | Ph | H | CH₂CH═CH₂ |
| C-466 | H | CH₂OH | H | CH₂CH═CH₂ |
| C-467 | H | CH(OH)CH₃ | H | CH₂CH═CH₂ |
| C-468 | H | CH₂SH | H | CH₂CH═CH₂ |
| C-469 | H | CH₂SCH₂CH₃ | H | CH₂CH═CH₂ |
| C-470 | H | CH₂CONH₂ | H | CH₂CH═CH₂ |
| C-471 | H | CH₂CH₂CONH₂ | H | CH₂CH═CH₂ |
| C-472 | H | CH₃ | CH₃ | CH₂CH═CH₂ |
| C-473 | H | CH₃CH₂ | CH₃ | CH₂CH═CH₂ |
| C-474 | H | n-C₃H₇ | CH₃ | CH₂CH═CH₂ |
| C-475 | H | i-C₃H₇ | CH₃ | CH₂CH═CH₂ |
| C-476 | H | n-C₄H₉ | CH₃ | CH₂CH═CH₂ |
| C-477 | H | i-C₄H₉ | CH₃ | CH₂CH═CH₂ |
| C-478 | H | s-C₄H₉ | CH₃ | CH₂CH═CH₂ |
| C-479 | H | t-C₄H₉ | CH₃ | CH₂CH═CH₂ |
| C-480 | H | CH₂Ph | CH₃ | CH₂CH═CH₂ |
| C-481 | H | Ph | CH₃ | CH₂CH═CH₂ |
| C-482 | H | CH₂OH | CH₃ | CH₂CH═CH₂ |
| C-483 | H | CH(OH)CH₃ | CH₃ | CH₂CH═CH₂ |
| C-484 | H | CH₂SH | CH₃ | CH₂CH═CH₂ |
| C-485 | H | CH₂SCH₂CH₃ | CH₃ | CH₂CH═CH₂ |
| C-486 | H | CH₂CONH₂ | CH₃ | CH₂CH═CH₂ |
| C-487 | H | CH₂CH₂CONH₂ | CH₃ | CH₂CH═CH₂ |
| C-488 | H | H | H | CH₂Ph |
| C-489 | H | CH₃ | H | CH₂Ph |
| C-490 | H | CH₃CH₂ | H | CH₂Ph |
| C-491 | H | n-C₃H₇ | H | CH₂Ph |
| C-492 | H | i-C₃H₇ | H | CH₂Ph |
| C-493 | H | n-C₄H₉ | H | CH₂Ph |
| C-494 | H | i-C₄H₉ | H | CH₂Ph |
| C-495 | H | s-C₄H₉ | H | CH₂Ph |
| C-496 | H | t-C₄H₉ | H | CH₂Ph |
| C-497 | H | CH₂Ph | H | CH₂Ph |
| C-498 | H | Ph | H | CH₂Ph |
| C-499 | H | CH₂OH | H | CH₂Ph |
| C-500 | H | CH(OH)CH₃ | H | CH₂Ph |
| C-501 | H | CH₂SH | H | CH₂Ph |
| C-502 | H | CH₂SCH₂CH₃ | H | CH₂Ph |
| C-503 | H | CH₂CONH₂ | H | CH₂Ph |
| C-504 | H | CH₂CH₂CONH₂ | H | CH₂Ph |
| C-505 | H | CH₃ | CH₃ | CH₂Ph |
| C-506 | H | CH₃CH₂ | CH₃ | CH₂Ph |
| C-507 | H | n-C₃H₇ | CH₃ | CH₂Ph |
| C-508 | H | i-C₃H₇ | CH₃ | CH₂Ph |
| C-509 | H | n-C₄H₉ | CH₃ | CH₂Ph |
| C-510 | H | i-C₄H₉ | CH₃ | CH₂Ph |
| C-511 | H | s-C₄H₉ | CH₃ | CH₂Ph |
| C-512 | H | t-C₄H₉ | CH₃ | CH₂Ph |
| C-513 | H | CH₂Ph | CH₃ | CH₂Ph |
| C-514 | H | Ph | CH₃ | CH₂Ph |
| C-515 | H | CH₂OH | CH₃ | CH₂Ph |
| C-516 | H | CH(OH)CH₃ | CH₃ | CH₂Ph |
| C-517 | H | CH₂SH | CH₃ | CH₂Ph |
| C-518 | H | CH₂SCH₂CH₃ | CH₃ | CH₂Ph |
| C-519 | H | CH₂CONH₂ | CH₃ | CH₂Ph |
| C-520 | H | CH₂CH₂CONH₂ | CH₃ | CH₂Ph |
| C-521 | H | H | H | CH₂(Ph-2-Cl) |
| C-522 | H | H | H | CH₂(Ph-3-Cl) |
| C-523 | H | H | H | CH₂(Ph-4-Cl) |
| C-524 | H | CH₃ | H | CH₂(Ph-2-Cl) |
| C-525 | H | CH₃ | H | CH₂(Ph-3-Cl) |
| C-526 | H | CH₃ | H | CH₂(Ph-4-Cl) |
| C-527 | H | CH₃ | CH₃ | CH₂(Ph-2-Cl) |
| C-528 | H | CH₃ | CH₃ | CH₂(Ph-3-Cl) |
| C-529 | H | CH₃ | CH₃ | CH₂(Ph-4-Cl) |
| C-530 | H | H | H | Ph |
| C-531 | H | CH₃ | H | Ph |
| C-532 | H | CH₃ | CH₃ | Ph |
| C-533 | H | H | H | 2-CH₃-4-C₃F₇—Ph |
| C-534 | H | CH₃ | H | 2-CH₃-4-C₃F₇—Ph |
| C-535 | H | CH₃ | CH₃ | 2-CH₃-4-C₃F₇—Ph |
| C-536 | H | H | H | CH₂CO₂CH₃ |
| C-537 | H | CH₃ | H | CH₂CO₂CH₃ |
| C-538 | H | CH₃ | CH₃ | CH₂CO₂CH₃ |
| C-539 | H | H | H | C(CH₃)₂CO₂CH₂Ph |
| C-540 | H | CH₃ | H | C(CH₃)₂CO₂CH₂Ph |
| C-541 | H | CH₃ | CH₃ | C(CH₃)₂CO₂CH₂Ph |

Table 4 of formula (Id):

| | |
|---|---|
| | (Id) |
| 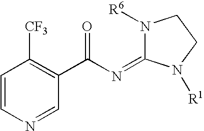 | |

| Compound | R¹ | R⁶ |
|---|---|---|
| D-1 | H | H |
| D-2 | CH₃ | H |
| D-3 | CH₃CH₂ | H |
| D-4 | n-C₃H₇ | H |
| D-5 | i-C₃H₇ | H |
| D-6 | n-C₄H₉ | H |
| D-7 | i-C₄H₉ | H |
| D-8 | s-C₄H₉ | H |
| D-9 | t-C₄H₉ | H |
| D-10 | CH₂CH═CH₂ | H |
| D-11 | CH₂Ph | H |
| D-12 | CH₂(2-chloropyridyl-5-yl) | H |
| D-13 | CH₂(2-chlorothiazole-5-yl) | H |
| D-14 | Ph | H |
| D-15 | 2-CH₃-4-C₃F₇—Ph | H |
| D-16 | CH₃ | CH₃ |
| D-17 | CH₃CH₂ | CH₃ |
| D-18 | n-C₃H₇ | CH₃ |
| D-19 | i-C₃H₇ | CH₃ |
| D-20 | n-C₄H₉ | CH₃ |
| D-21 | i-C₄H₉ | CH₃ |
| D-22 | s-C₄H₉ | CH₃ |
| D-23 | t-C₄H₉ | CH₃ |
| D-24 | CH₂CH═CH₂ | CH₃ |
| D-25 | CH₂Ph | CH₃ |
| D-26 | CH₂(2-chloropyridyl-5-yl) | CH₃ |
| D-27 | CH₂(2-chlorothiazole-5-yl) | CH₃ |
| D-28 | Ph | CH₃ |
| D-29 | 2-CH₃-4-C₃F₇—Ph | CH₃ |
| D-30 | CH₃ | C₂H₅ |
| D-31 | CH₃CH₂ | C₂H₅ |
| D-32 | n-C₃H₇ | C₂H₅ |
| D-33 | i-C₃H₇ | C₂H₅ |
| D-34 | n-C₄H₉ | C₂H₅ |
| D-35 | i-C₄H₉ | C₂H₅ |
| D-36 | s-C₄H₉ | C₂H₅ |
| D-37 | t-C₄H₉ | C₂H₅ |
| D-38 | CH₂CH═CH₂ | C₂H₅ |
| D-39 | CH₂Ph | C₂H₅ |
| D-40 | CH₂(2-chloropyridyl-5-yl) | C₂H₅ |
| D-41 | CH₂(2-chlorothiazole-5-yl) | C₂H₅ |
| D-42 | Ph | C₂H₅ |
| D-43 | 2-CH₃-4-C₃F₇—Ph | C₂H₅ |
| D-44 | CH₃ | CH₂CH═CH₂ |
| D-45 | CH₃CH₂ | CH₂CH═CH₂ |
| D-46 | n-C₃H₇ | CH₂CH═CH₂ |
| D-47 | i-C₃H₇ | CH₂CH═CH₂ |
| D-48 | n-C₄H₉ | CH₂CH═CH₂ |
| D-49 | i-C₄H₉ | CH₂CH═CH₂ |
| D-50 | s-C₄H₉ | CH₂CH═CH₂ |
| D-51 | t-C₄H₉ | CH₂CH═CH₂ |
| D-52 | CH₂CH═CH₂ | CH₂CH═CH₂ |
| D-53 | CH₂Ph | CH₂CH═CH₂ |
| D-54 | CH₂(2-chloropyridyl-5-yl) | CH₂CH═CH₂ |
| D-55 | CH₂(2-chlorothiazole-5-yl) | CH₂CH═CH₂ |
| D-56 | Ph | CH₂CH═CH₂ |
| D-57 | 2-CH₃-4-C₃F₇—Ph | CH₂CH═CH₂ |
| D-58 | CH₃ | CH₂Ph |
| D-59 | CH₃CH₂ | CH₂Ph |
| D-60 | n-C₃H₇ | CH₂Ph |
| D-61 | i-C₃H₇ | CH₂Ph |
| D-62 | n-C₄H₉ | CH₂Ph |
| D-63 | i-C₄H₉ | CH₂Ph |
| D-64 | s-C₄H₉ | CH₂Ph |
| D-65 | t-C₄H₉ | CH₂Ph |
| D-66 | CH₂CH═CH₂ | CH₂Ph |
| D-67 | CH₂Ph | CH₂Ph |
| D-68 | CH₂(2-chloropyridyl-5-yl) | CH₂Ph |
| D-69 | CH₂(2-chlorothiazole-5-yl) | CH₂Ph |
| D-70 | Ph | CH₂Ph |
| D-71 | 2-CH₃-4-C₃F₇—Ph | CH₂Ph |
| D-72 | CH₃ | Ph |
| D-73 | CH₃CH₂ | Ph |
| D-74 | n-C₃H₇ | Ph |
| D-75 | i-C₃H₇ | Ph |
| D-76 | n-C₄H₉ | Ph |
| D-77 | i-C₄H₉ | Ph |
| D-78 | s-C₄H₉ | Ph |
| D-79 | t-C₄H₉ | Ph |
| D-80 | CH₂CH═CH₂ | Ph |
| D-81 | CH₂Ph | Ph |
| D-82 | CH₂(2-chloropyridyl-5-yl) | Ph |
| D-83 | CH₂(2-chlorothiazole-5-yl) | Ph |
| D-84 | Ph | Ph |

TABLE 5
Compounds of formula (Ie):

| | |
|---|---|
| | (Ie) |
| 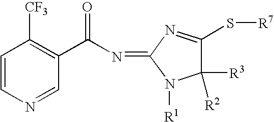 | |

| Compound | R¹ | R² | R³ | R⁷ |
|---|---|---|---|---|
| E-1 | H | H | H | CH₃ |
| E-2 | CH₃ | H | H | CH₃ |
| E-3 | CH₂CH₃ | H | H | CH₃ |
| E-4 | CH₂CH═CH₂ | H | H | CH₃ |
| E-5 | CH₂Ph | H | H | CH₃ |
| E-6 | Ph | H | H | CH₃ |
| E-7 | COCH₃ | H | H | CH₃ |
| E-8 | COPh | H | H | CH₃ |
| E-9 | H | CH₃ | H | CH₃ |
| E-10 | CH₃ | CH₃ | H | CH₃ |
| E-11 | CH₂CH₃ | CH₃ | H | CH₃ |
| E-12 | CH₂CH═CH₂ | CH₃ | H | CH₃ |
| E-13 | CH₂Ph | CH₃ | H | CH₃ |
| E-14 | Ph | CH₃ | H | CH₃ |
| E-15 | COCH₃ | CH₃ | H | CH₃ |
| E-16 | COPh | CH₃ | H | CH₃ |
| E-17 | H | CH₃CH₂ | H | CH₃ |
| E-18 | CH₃ | CH₃CH₂ | H | CH₃ |
| E-19 | CH₂CH₃ | CH₃CH₂ | H | CH₃ |
| E-20 | CH₂CH═CH₂ | CH₃CH₂ | H | CH₃ |
| E-21 | CH₂Ph | CH₃CH₂ | H | CH₃ |
| E-22 | Ph | CH₃CH₂ | H | CH₃ |
| E-23 | COCH₃ | CH₃CH₂ | H | CH₃ |
| E-24 | COPh | CH₃CH₂ | H | CH₃ |
| E-25 | H | n-C₃H₇ | H | CH₃ |
| E-26 | CH₃ | n-C₃H₇ | H | CH₃ |
| E-27 | CH₂CH₃ | n-C₃H₇ | H | CH₃ |
| E-28 | CH₂CH═CH₂ | n-C₃H₇ | H | CH₃ |
| E-29 | CH₂Ph | n-C₃H₇ | H | CH₃ |
| E-30 | Ph | n-C₃H₇ | H | CH₃ |
| E-31 | COCH₃ | n-C₃H₇ | H | CH₃ |
| E-32 | COPh | n-C₃H₇ | H | CH₃ |
| E-33 | H | i-C₃H₇ | H | CH₃ |
| E-34 | CH₃ | i-C₃H₇ | H | CH₃ |
| E-35 | CH₂CH₃ | i-C₃H₇ | H | CH₃ |
| E-36 | CH₂CH═CH₂ | i-C₃H₇ | H | CH₃ |
| E-37 | CH₂Ph | i-C₃H₇ | H | CH₃ |
| E-38 | Ph | i-C₃H₇ | H | CH₃ |
| E-39 | COCH₃ | i-C₃H₇ | H | CH₃ |
| E-40 | COPh | i-C₃H₇ | H | CH₃ |
| E-41 | H | n-C₄H₉ | H | CH₃ |
| E-42 | CH₃ | n-C₄H₉ | H | CH₃ |
| E-43 | CH₂CH₃ | n-C₄H₉ | H | CH₃ |
| E-44 | CH₂CH═CH₂ | n-C₄H₉ | H | CH₃ |
| E-45 | CH₂Ph | n-C₄H₉ | H | CH₃ |
| E-46 | Ph | n-C₄H₉ | H | CH₃ |
| E-47 | COCH₃ | n-C₄H₉ | H | CH₃ |
| E-48 | COPh | n-C₄H₉ | H | CH₃ |
| E-49 | H | i-C₄H₉ | H | CH₃ |
| E-50 | CH₃ | i-C₄H₉ | H | CH₃ |
| E-51 | CH₂CH₃ | i-C₄H₉ | H | CH₃ |
| E-52 | CH₂CH═CH₂ | i-C₄H₉ | H | CH₃ |
| E-53 | CH₂Ph | i-C₄H₉ | H | CH₃ |
| E-54 | Ph | i-C₄H₉ | H | CH₃ |
| E-55 | COCH₃ | i-C₄H₉ | H | CH₃ |
| E-56 | COPh | i-C₄H₉ | H | CH₃ |
| E-57 | H | s-C₄H₉ | H | CH₃ |
| E-58 | CH₃ | s-C₄H₉ | H | CH₃ |
| E-59 | CH₂CH₃ | s-C₄H₉ | H | CH₃ |
| E-60 | CH₂CH═CH₂ | s-C₄H₉ | H | CH₃ |
| E-61 | CH₂Ph | s-C₄H₉ | H | CH₃ |
| E-62 | Ph | s-C₄H₉ | H | CH₃ |
| E-63 | COCH₃ | s-C₄H₉ | H | CH₃ |
| E-64 | COPh | s-C₄H₉ | H | CH₃ |
| E-65 | H | t-C₄H₉ | H | CH₃ |
| E-66 | CH₃ | t-C₄H₉ | H | CH₃ |
| E-67 | CH₂CH₃ | t-C₄H₉ | H | CH₃ |
| E-68 | CH₂CH═CH₂ | t-C₄H₉ | H | CH₃ |
| E-69 | CH₂Ph | t-C₄H₉ | H | CH₃ |
| E-70 | Ph | t-C₄H₉ | H | CH₃ |
| E-71 | COCH₃ | t-C₄H₉ | H | CH₃ |
| E-72 | COPh | t-C₄H₉ | H | CH₃ |
| E-73 | H | CH₂Ph | H | CH₃ |
| E-74 | CH₃ | CH₂Ph | H | CH₃ |
| E-75 | CH₂CH₃ | CH₂Ph | H | CH₃ |
| E-76 | CH₂CH═CH₂ | CH₂Ph | H | CH₃ |
| E-77 | CH₂Ph | CH₂Ph | H | CH₃ |
| E-78 | Ph | CH₂Ph | H | CH₃ |
| E-79 | COCH₃ | CH₂Ph | H | CH₃ |
| E-80 | COPh | CH₂Ph | H | CH₃ |
| E-81 | H | Ph | H | CH₃ |
| E-82 | CH₃ | Ph | H | CH₃ |
| E-83 | CH₂CH₃ | Ph | H | CH₃ |
| E-84 | CH₂CH═CH₂ | Ph | H | CH₃ |
| E-85 | CH₂Ph | Ph | H | CH₃ |
| E-86 | Ph | Ph | H | CH₃ |
| E-87 | COCH₃ | Ph | H | CH₃ |
| E-88 | COPh | Ph | H | CH₃ |
| E-89 | H | CH₂OH | H | CH₃ |
| E-90 | CH₃ | CH₂OH | H | CH₃ |
| E-91 | CH₂CH₃ | CH₂OH | H | CH₃ |
| E-92 | CH₂CH═CH₂ | CH₂OH | H | CH₃ |
| E-93 | CH₂Ph | CH₂OH | H | CH₃ |
| E-94 | Ph | CH₂OH | H | CH₃ |
| E-95 | COCH₃ | CH₂OH | H | CH₃ |
| E-96 | COPh | CH₂OH | H | CH₃ |
| E-97 | H | CH(OH)CH₃ | H | CH₃ |
| E-98 | CH₃ | CH(OH)CH₃ | H | CH₃ |
| E-99 | CH₂CH₃ | CH(OH)CH₃ | H | CH₃ |
| E-100 | CH₂CH═CH₂ | CH(OH)CH₃ | H | CH₃ |
| E-101 | CH₂Ph | CH(OH)CH₃ | H | CH₃ |
| E-102 | Ph | CH(OH)CH₃ | H | CH₃ |
| E-103 | COCH₃ | CH(OH)CH₃ | H | CH₃ |
| E-104 | COPh | CH(OH)CH₃ | H | CH₃ |
| E-105 | H | CH₂SH | H | CH₃ |
| E-106 | CH₃ | CH₂SH | H | CH₃ |
| E-107 | CH₂CH₃ | CH₂SH | H | CH₃ |
| E-108 | CH₂CH═CH₂ | CH₂SH | H | CH₃ |
| E-109 | CH₂Ph | CH₂SH | H | CH₃ |
| E-110 | Ph | CH₂SH | H | CH₃ |
| E-111 | COCH₃ | CH₂SH | H | CH₃ |
| E-112 | COPh | CH₂SH | H | CH₃ |
| E-113 | H | CH₂SCH₂CH₃ | H | CH₃ |
| E-114 | CH₃ | CH₂SCH₂CH₃ | H | CH₃ |
| E-115 | CH₂CH₃ | CH₂SCH₂CH₃ | H | CH₃ |
| E-116 | CH₂CH═CH₂ | CH₂SCH₂CH₃ | H | CH₃ |
| E-117 | CH₂Ph | CH₂SCH₂CH₃ | H | CH₃ |
| E-118 | Ph | CH₂SCH₂CH₃ | H | CH₃ |
| E-119 | COCH₃ | CH₂SCH₂CH₃ | H | CH₃ |
| E-120 | COPh | CH₂SCH₂CH₃ | H | CH₃ |
| E-121 | H | CH₂CONH₂ | H | CH₃ |
| E-122 | CH₃ | CH₂CONH₂ | H | CH₃ |
| E-123 | CH₂CH₃ | CH₂CONH₂ | H | CH₃ |
| E-124 | CH₂CH═CH₂ | CH₂CONH₂ | H | CH₃ |
| E-125 | CH₂Ph | CH₂CONH₂ | H | CH₃ |
| E-126 | Ph | CH₂CONH₂ | H | CH₃ |
| E-127 | COCH₃ | CH₂CONH₂ | H | CH₃ |
| E-128 | COPh | CH₂CONH₂ | H | CH₃ |
| E-129 | H | CH₃ | CH₃ | CH₃ |
| E-130 | CH₃ | CH₃ | CH₃ | CH₃ |
| E-131 | CH₂CH₃ | CH₃ | CH₃ | CH₃ |
| E-132 | CH₂CH═CH₂ | CH₃ | CH₃ | CH₃ |
| E-133 | CH₂Ph | CH₃ | CH₃ | CH₃ |
| E-134 | Ph | CH₃ | CH₃ | CH₃ |
| E-135 | COCH₃ | CH₃ | CH₃ | CH₃ |
| E-136 | COPh | CH₃ | CH₃ | CH₃ |
| E-137 | H | CH₃CH₂ | CH₃ | CH₃ |
| E-138 | CH₃ | CH₃CH₂ | CH₃ | CH₃ |
| E-139 | CH₂CH₃ | CH₃CH₂ | CH₃ | CH₃ |
| E-140 | CH₂CH═CH₂ | CH₃CH₂ | CH₃ | CH₃ |
| E-141 | CH₂Ph | CH₃CH₂ | CH₃ | CH₃ |
| E-142 | Ph | CH₃CH₂ | CH₃ | CH₃ |
| E-143 | COCH₃ | CH₃CH₂ | CH₃ | CH₃ |
| E-144 | COPh | CH₃CH₂ | CH₃ | CH₃ |
| E-145 | H | n-C₃H₇ | CH₃ | CH₃ |
| E-146 | CH₃ | n-C₃H₇ | CH₃ | CH₃ |
| E-147 | CH₂CH₃ | n-C₃H₇ | CH₃ | CH₃ |
| E-148 | CH₂CH═CH₂ | n-C₃H₇ | CH₃ | CH₃ |
| E-149 | CH₂Ph | n-C₃H₇ | CH₃ | CH₃ |
| E-150 | Ph | n-C₃H₇ | CH₃ | CH₃ |
| E-151 | COCH₃ | n-C₃H₇ | CH₃ | CH₃ |
| E-152 | COPh | n-C₃H₇ | CH₃ | CH₃ |
| E-153 | H | i-C₃H₇ | CH₃ | CH₃ |
| E-154 | CH₃ | i-C₃H₇ | CH₃ | CH₃ |
| E-155 | CH₂CH₃ | i-C₃H₇ | CH₃ | CH₃ |
| E-156 | CH₂CH═CH₂ | i-C₃H₇ | CH₃ | CH₃ |
| E-157 | CH₂Ph | i-C₃H₇ | CH₃ | CH₃ |
| E-158 | Ph | i-C₃H₇ | CH₃ | CH₃ |
| E-159 | COCH₃ | i-C₃H₇ | CH₃ | CH₃ |
| E-160 | COPh | i-C₃H₇ | CH₃ | CH₃ |
| E-161 | H | n-C₄H₉ | CH₃ | CH₃ |
| E-162 | CH₃ | n-C₄H₉ | CH₃ | CH₃ |
| E-163 | CH₂CH₃ | n-C₄H₉ | CH₃ | CH₃ |
| E-164 | CH₂CH═CH₂ | n-C₄H₉ | CH₃ | CH₃ |
| E-165 | CH₂Ph | n-C₄H₉ | CH₃ | CH₃ |
| E-166 | Ph | n-C₄H₉ | CH₃ | CH₃ |
| E-167 | COCH₃ | n-C₄H₉ | CH₃ | CH₃ |
| E-168 | COPh | n-C₄H₉ | CH₃ | CH₃ |
| E-169 | H | i-C₄H₉ | CH₃ | CH₃ |
| E-170 | CH₃ | i-C₄H₉ | CH₃ | CH₃ |
| E-171 | CH₂CH₃ | i-C₄H₉ | CH₃ | CH₃ |
| E-172 | CH₂CH═CH₂ | i-C₄H₉ | CH₃ | CH₃ |
| E-173 | CH₂Ph | i-C₄H₉ | CH₃ | CH₃ |
| E-174 | Ph | i-C₄H₉ | CH₃ | CH₃ |
| E-175 | COCH₃ | i-C₄H₉ | CH₃ | CH₃ |
| E-176 | COPh | i-C₄H₉ | CH₃ | CH₃ |
| E-177 | H | s-C₄H₉ | CH₃ | CH₃ |
| E-178 | CH₃ | s-C₄H₉ | CH₃ | CH₃ |
| E-179 | CH₂CH₃ | s-C₄H₉ | CH₃ | CH₃ |
| E-180 | CH₂CH═CH₂ | s-C₄H₉ | CH₃ | CH₃ |
| E-181 | CH₂Ph | s-C₄H₉ | CH₃ | CH₃ |
| E-182 | Ph | s-C₄H₉ | CH₃ | CH₃ |
| E-183 | COCH₃ | s-C₄H₉ | CH₃ | CH₃ |
| E-184 | COPh | s-C₄H₉ | CH₃ | CH₃ |
| E-185 | H | t-C₄H₉ | CH₃ | CH₃ |
| E-186 | CH₃ | t-C₄H₉ | CH₃ | CH₃ |
| E-187 | CH₂CH₃ | t-C₄H₉ | CH₃ | CH₃ |
| E-188 | CH₂CH═CH₂ | t-C₄H₉ | CH₃ | CH₃ |
| E-189 | CH₂Ph | t-C₄H₉ | CH₃ | CH₃ |
| E-190 | Ph | t-C₄H₉ | CH₃ | CH₃ |
| E-191 | COCH₃ | t-C₄H₉ | CH₃ | CH₃ |
| E-192 | COPh | t-C₄H₉ | CH₃ | CH₃ |
| E-193 | H | CH₂Ph | CH₃ | CH₃ |
| E-194 | CH₃ | CH₂Ph | CH₃ | CH₃ |
| E-195 | CH₂CH₃ | CH₂Ph | CH₃ | CH₃ |
| E-196 | CH₂CH═CH₂ | CH₂Ph | CH₃ | CH₃ |
| E-197 | CH₂Ph | CH₂Ph | CH₃ | CH₃ |
| E-198 | Ph | CH₂Ph | CH₃ | CH₃ |
| E-199 | COCH₃ | CH₂Ph | CH₃ | CH₃ |
| E-200 | COPh | CH₂Ph | CH₃ | CH₃ |
| E-201 | H | Ph | CH₃ | CH₃ |
| E-202 | CH₃ | Ph | CH₃ | CH₃ |
| E-203 | CH₂CH₃ | Ph | CH₃ | CH₃ |
| E-204 | CH₂CH═CH₂ | Ph | CH₃ | CH₃ |
| E-205 | CH₂Ph | Ph | CH₃ | CH₃ |
| E-206 | Ph | Ph | CH₃ | CH₃ |
| E-207 | COCH₃ | Ph | CH₃ | CH₃ |
| E-208 | COPh | Ph | CH₃ | CH₃ |
| E-209 | H | CH₂OH | CH₃ | CH₃ |
| E-210 | CH₃ | CH₂OH | CH₃ | CH₃ |
| E-211 | CH₂CH₃ | CH₂OH | CH₃ | CH₃ |
| E-212 | CH₂CH═CH₂ | CH₂OH | CH₃ | CH₃ |
| E-213 | CH₂Ph | CH₂OH | CH₃ | CH₃ |
| E-214 | Ph | CH₂OH | CH₃ | CH₃ |
| E-215 | COCH₃ | CH₂OH | CH₃ | CH₃ |
| E-216 | COPh | CH₂OH | CH₃ | CH₃ |
| E-217 | H | CH(OH)CH₃ | CH₃ | CH₃ |
| E-218 | CH₃ | CH(OH)CH₃ | CH₃ | CH₃ |
| E-219 | CH₂CH₃ | CH(OH)CH₃ | CH₃ | CH₃ |
| E-220 | CH₂CH═CH₂ | CH(OH)CH₃ | CH₃ | CH₃ |
| E-221 | CH₂Ph | CH(OH)CH₃ | CH₃ | CH₃ |
| E-222 | Ph | CH(OH)CH₃ | CH₃ | CH₃ |
| E-223 | COCH₃ | CH(OH)CH₃ | CH₃ | CH₃ |
| E-224 | COPh | CH(OH)CH₃ | CH₃ | CH₃ |
| E-225 | H | CH₂SH | CH₃ | CH₃ |
| E-226 | CH₃ | CH₂SH | CH₃ | CH₃ |
| E-227 | CH₂CH₃ | CH₂SH | CH₃ | CH₃ |
| E-228 | CH₂CH═CH₂ | CH₂SH | CH₃ | CH₃ |
| E-229 | CH₂Ph | CH₂SH | CH₃ | CH₃ |
| E-230 | Ph | CH₂SH | CH₃ | CH₃ |
| E-231 | COCH₃ | CH₂SH | CH₃ | CH₃ |
| E-232 | COPh | CH₂SH | CH₃ | CH₃ |
| E-233 | H | CH₂SCH₂CH₃ | CH₃ | CH₃ |
| E-234 | CH₃ | CH₂SCH₂CH₃ | CH₃ | CH₃ |
| E-235 | CH₂CH₃ | CH₂SCH₂CH₃ | CH₃ | CH₃ |
| E-236 | CH₂CH═CH₂ | CH₂SCH₂CH₃ | CH₃ | CH₃ |
| E-237 | CH₂Ph | CH₂SCH₂CH₃ | CH₃ | CH₃ |
| E-238 | Ph | CH₂SCH₂CH₃ | CH₃ | CH₃ |
| E-239 | COCH₃ | CH₂SCH₂CH₃ | CH₃ | CH₃ |
| E-240 | COPh | CH₂SCH₂CH₃ | CH₃ | CH₃ |
| E-241 | H | CH₂CONH₂ | CH₃ | CH₃ |
| E-242 | CH₃ | CH₂CONH₂ | CH₃ | CH₃ |
| E-243 | CH₂CH₃ | CH₂CONH₂ | CH₃ | CH₃ |
| E-244 | CH₂CH═CH₂ | CH₂CONH₂ | CH₃ | CH₃ |
| E-245 | CH₂Ph | CH₂CONH₂ | CH₃ | CH₃ |
| E-246 | Ph | CH₂CONH₂ | CH₃ | CH₃ |
| E-247 | COCH₃ | CH₂CONH₂ | CH₃ | CH₃ |
| E-248 | COPh | CH₂CONH₂ | CH₃ | CH₃ |
| E-249 | H | CH₂CH₂CONH₂ | CH₃ | CH₃ |
| E-250 | CH₃ | CH₂CH₂CONH₂ | CH₃ | CH₃ |
| E-251 | CH₂CH₃ | CH₂CH₂CONH₂ | CH₃ | CH₃ |
| E-252 | CH₂CH═CH₂ | CH₂CH₂CONH₂ | CH₃ | CH₃ |
| E-253 | CH₂Ph | CH₂CH₂CONH₂ | CH₃ | CH₃ |
| E-254 | Ph | CH₂CH₂CONH₂ | CH₃ | CH₃ |
| E-255 | COCH₃ | CH₂CH₂CONH₂ | CH₃ | CH₃ |
| E-256 | COPh | CH₂CH₂CONH₂ | CH₃ | CH₃ |
| E-257 | CH₃ | CH₃CH₂ | CH₃CH₂ | CH₃ |
| E-258 | CH₂CH₃ | CH₃CH₂ | CH₃CH₂ | CH₃ |
| E-259 | CH₂CH═CH₂ | CH₃CH₂ | CH₃CH₂ | CH₃ |
| E-260 | CH₂Ph | CH₃CH₂ | CH₃CH₂ | CH₃ |
| E-261 | Ph | CH₃CH₂ | CH₃CH₂ | CH₃ |
| E-262 | COCH₃ | CH₃CH₂ | CH₃CH₂ | CH₃ |
| E-263 | COPh | CH₃CH₂ | CH₃CH₂ | CH₃ |
| E-264 | H | n-C₃H₇ | CH₃CH₂ | CH₃ |
| E-265 | CH₃ | n-C₃H₇ | CH₃CH₂ | CH₃ |
| E-266 | CH₂CH₃ | n-C₃H₇ | CH₃CH₂ | CH₃ |
| E-267 | CH₂CH═CH₂ | n-C₃H₇ | CH₃CH₂ | CH₃ |
| E-268 | CH₂Ph | n-C₃H₇ | CH₃CH₂ | CH₃ |
| E-269 | Ph | n-C₃H₇ | CH₃CH₂ | CH₃ |
| E-270 | COCH₃ | n-C₃H₇ | CH₃CH₂ | CH₃ |
| E-271 | COPh | n-C₃H₇ | CH₃CH₂ | CH₃ |
| E-272 | H | i-C₃H₇ | CH₃CH₂ | CH₃ |
| E-273 | CH₃ | i-C₃H₇ | CH₃CH₂ | CH₃ |
| E-274 | CH₂CH₃ | i-C₃H₇ | CH₃CH₂ | CH₃ |
| E-275 | CH₂CH═CH₂ | i-C₃H₇ | CH₃CH₂ | CH₃ |
| E-276 | CH₂Ph | i-C₃H₇ | CH₃CH₂ | CH₃ |
| E-277 | Ph | i-C₃H₇ | CH₃CH₂ | CH₃ |
| E-278 | COCH₃ | i-C₃H₇ | CH₃CH₂ | CH₃ |
| E-279 | COPh | i-C₃H₇ | CH₃CH₂ | CH₃ |
| E-280 | H | n-C₄H₉ | CH₃CH₂ | CH₃ |
| E-281 | CH₃ | n-C₄H₉ | CH₃CH₂ | CH₃ |
| E-282 | CH₂CH₃ | n-C₄H₉ | CH₃CH₂ | CH₃ |
| E-283 | CH₂CH═CH₂ | n-C₄H₉ | CH₃CH₂ | CH₃ |
| E-284 | CH₂Ph | n-C₄H₉ | CH₃CH₂ | CH₃ |
| E-285 | Ph | n-C₄H₉ | CH₃CH₂ | CH₃ |
| E-286 | COCH₃ | n-C₄H₉ | CH₃CH₂ | CH₃ |
| E-287 | COPh | n-C₄H₉ | CH₃CH₂ | CH₃ |
| E-288 | H | t-C₄H₉ | CH₃CH₂ | CH₃ |
| E-289 | CH₃ | t-C₄H₉ | CH₃CH₂ | CH₃ |
| E-290 | CH₂CH₃ | t-C₄H₉ | CH₃CH₂ | CH₃ |
| E-291 | CH₂CH═CH₂ | t-C₄H₉ | CH₃CH₂ | CH₃ |
| E-292 | CH₂Ph | t-C₄H₉ | CH₃CH₂ | CH₃ |
| E-293 | Ph | t-C₄H₉ | CH₃CH₂ | CH₃ |
| E-294 | COCH₃ | t-C₄H₉ | CH₃CH₂ | CH₃ |
| E-295 | COPh | t-C₄H₉ | CH₃CH₂ | CH₃ |
| E-296 | H | CH₂Ph | CH₃CH₂ | CH₃ |
| E-297 | CH₃ | CH₂Ph | CH₃CH₂ | CH₃ |
| E-298 | CH₂CH₃ | CH₂Ph | CH₃CH₂ | CH₃ |
| E-299 | CH₂CH═CH₂ | CH₂Ph | CH₃CH₂ | CH₃ |
| E-300 | CH₂Ph | CH₂Ph | CH₃CH₂ | CH₃ |
| E-301 | Ph | CH₂Ph | CH₃CH₂ | CH₃ |
| E-302 | COCH₃ | CH₂Ph | CH₃CH₂ | CH₃ |
| E-303 | COPh | CH₂Ph | CH₃CH₂ | CH₃ |
| E-304 | H | CH₂CH₂ | | CH₃ |
| E-305 | CH₃ | CH₂CH₂ | | CH₃ |
| E-306 | CH₂CH═CH₂ | CH₂CH₂ | | CH₃ |
| E-307 | CH₂Ph | CH₂CH₂ | | CH₃ |
| E-308 | Ph | CH₂CH₂ | | CH₃ |
| E-309 | COCH₃ | CH₂CH₂ | | CH₃ |
| E-310 | COPh | CH₂CH₂ | | CH₃ |
| E-311 | H | CH₂H₂CH₂ | | CH₃ |
| E-312 | CH₃ | CH₂H₂CH₂ | | CH₃ |
| E-313 | CH₂CH═CH₂ | CH₂H₂CH₂ | | CH₃ |
| E-314 | CH₂Ph | CH₂H₂CH₂ | | CH₃ |
| E-315 | Ph | CH₂H₂CH₂ | | CH₃ |
| E-316 | COCH₃ | CH₂H₂CH₂ | | CH₃ |
| E-317 | COPh | CH₂H₂CH₂ | | CH₃ |
| E-318 | H | CH₂CH₂CH₂CH₂ | | CH₃ |
| E-319 | CH₃ | CH₂CH₂CH₂CH₂ | | CH₃ |
| E-320 | CH₂CH═CH₂ | CH₂CH₂CH₂CH₂ | | CH₃ |
| E-321 | CH₂Ph | CH₂CH₂CH₂CH₂ | | CH₃ |
| E-322 | Ph | CH₂CH₂CH₂CH₂ | | CH₃ |
| E-323 | COCH₃ | CH₂CH₂CH₂CH₂ | | CH₃ |
| E-324 | COPh | CH₂CH₂CH₂CH₂ | | CH₃ |
| E-325 | H | CH₂CH₂CH₂CH₂CH₂ | | CH₃ |
| E-326 | CH₃ | CH₂CH₂CH₂CH₂CH₂ | | CH₃ |
| E-327 | CH₂CH═CH₂ | CH₂CH₂CH₂CH₂CH₂ | | CH₃ |
| E-328 | CH₂Ph | CH₂CH₂CH₂CH₂CH₂ | | CH₃ |
| E-329 | Ph | CH₂CH₂CH₂CH₂CH₂ | | CH₃ |
| E-330 | COCH₃ | CH₂CH₂CH₂CH₂CH₂ | | CH₃ |
| E-331 | COPh | CH₂CH₂CH₂CH₂CH₂ | | CH₃ |
| E-332 | H | H | H | CH₃CH₂ |
| E-333 | CH₃ | H | H | CH₃CH₂ |
| E-334 | CH₂CH₃ | H | H | CH₃CH₂ |
| E-335 | CH₂CH═CH₂ | H | H | CH₃CH₂ |
| E-336 | CH₂Ph | H | H | CH₃CH₂ |
| E-337 | Ph | H | H | CH₃CH₂ |
| E-338 | COCH₃ | H | H | CH₃CH₂ |
| E-339 | COPh | H | H | CH₃CH₂ |
| E-340 | H | CH₃ | H | CH₃CH₂ |
| E-341 | CH₃ | CH₃ | H | CH₃CH₂ |
| E-342 | CH₂CH₃ | CH₃ | H | CH₃CH₂ |
| E-343 | CH₂CH═CH₂ | CH₃ | H | CH₃CH₂ |
| E-344 | CH₂Ph | CH₃ | H | CH₃CH₂ |
| E-345 | Ph | CH₃ | H | CH₃CH₂ |
| E-346 | COCH₃ | CH₃ | H | CH₃CH₂ |
| E-347 | COPh | CH₃ | H | CH₃CH₂ |
| E-348 | H | CH₃CH₂ | H | CH₃CH₂ |
| E-349 | CH₃ | CH₃CH₂ | H | CH₃CH₂ |
| E-350 | CH₂CH₃ | CH₃CH₂ | H. | CH₃CH₂ |
| E-351 | CH₂CH═CH₂ | CH₃CH₂ | H | CH₃CH₂ |
| E-352 | CH₂Ph | CH₃CH₂ | H | CH₃CH₂ |
| E-353 | Ph | CH₃CH₂ | H | CH₃CH₂ |
| E-354 | COCH₃ | CH₃CH₂ | H | CH₃CH₂ |
| E-355 | COPh | n-C₃H₇ | H | CH₃CH₂ |
| E-356 | H | n-C₃H₇ | H | CH₃CH₂ |
| E-357 | CH₃ | n-C₃H₇ | H | CH₃CH₂ |
| E-358 | CH₂CH₃ | n-C₃H₇ | H | CH₃CH₂ |
| E-359 | CH₂CH═CH₂ | n-C₃H₇ | H | CH₃CH₂ |
| E-360 | CH₂Ph | n-C₃H₇ | H | CH₃CH₂ |
| E-361 | Ph | n-C₃H₇ | H | CH₃CH₂ |
| E-362 | COCH₃ | n-C₃H₇ | H | CH₃CH₂ |
| E-363 | COPh | n-C₄H₉ | H | CH₃CH₂ |
| E-364 | H | i-C₃H₇ | H | CH₃CH₂ |
| E-365 | CH₃ | i-C₃H₇ | H | CH₃CH₂ |
| E-366 | CH₂CH₃ | i-C₃H₇ | H | CH₃CH₂ |
| E-367 | CH₂CH═CH₂ | i-C₃H₇ | H | CH₃CH₂ |
| E-368 | CH₂Ph | i-C₃H₇ | H | CH₃CH₂ |
| E-369 | Ph | i-C₃H₇ | H | CH₃CH₂ |
| E-370 | COCH₃ | i-C₃H₇ | H | CH₃CH₂ |
| E-371 | COPh | i-C₃H₇ | H | CH₃CH₂ |
| E-372 | H | CH₂Ph | H | CH₃CH₂ |
| E-373 | CH₃ | CH₂Ph | H | CH₃CH₂ |
| E-374 | CH₂CH₃ | CH₂Ph | H | CH₃CH₂ |
| E-375 | CH₂CH═CH₂ | CH₂Ph | H | CH₃CH₂ |
| E-376 | CH₂Ph | CH₂Ph | H | CH₃CH₂ |
| E-377 | Ph | CH₂Ph | H | CH₃CH₂ |
| E-378 | COCH₃ | CH₂Ph | H | CH₃CH₂ |
| E-379 | COPh | CH₂Ph | H | CH₃CH₂ |
| E-380 | H | CH₃ | CH₃ | CH₃CH₂ |
| E-381 | CH₃ | CH₃ | CH₃ | CH₃CH₂ |
| E-382 | CH₂CH₃ | CH₃ | CH₃ | CH₃CH₂ |
| E-383 | CH₂CH═CH₂ | CH₃ | CH₃ | CH₃CH₂ |
| E-384 | CH₂Ph | CH₃ | CH₃ | CH₃CH₂ |
| E-385 | Ph | CH₃ | CH₃ | CH₃CH₂ |
| E-386 | COCH₃ | CH₃ | CH₃ | CH₃CH₂ |
| E-387 | COPh | CH₃ | CH₃ | CH₃CH₂ |
| E-388 | H | CH₃CH₂ | CH₃ | CH₃CH₂ |
| E-389 | CH₃ | CH₃CH₂ | CH₃ | CH₃CH₂ |
| E-390 | CH₂CH₃ | CH₃CH₂ | CH₃ | CH₃CH₂ |
| E-391 | CH₂CH═CH₂ | CH₃CH₂ | CH₃ | CH₃CH₂ |
| E-392 | CH₂Ph | CH₃CH₂ | CH₃ | CH₃CH₂ |
| E-393 | Ph | CH₃CH₂ | CH₃ | CH₃CH₂ |
| E-394 | COCH₃ | CH₃CH₂ | CH₃ | CH₃CH₂ |
| E-395 | COPh | n-C₃H₇ | CH₃ | CH₃CH₂ |
| E-396 | H | n-C₃H₇ | CH₃ | CH₃CH₂ |
| E-397 | CH₃ | n-C₃H₇ | CH₃ | CH₃CH₂ |
| E-398 | CH₂CH₃ | n-C₃H₇ | CH₃ | CH₃CH₂ |
| E-399 | CH₂CH═CH₂ | n-C₃H₇ | CH₃ | CH₃CH₂ |
| E-400 | CH₂Ph | n-C₃H₇ | CH₃ | CH₃CH₂ |
| E-401 | Ph | n-C₃H₇ | CH₃ | CH₃CH₂ |
| E-402 | COCH₃ | n-C₃H₇ | CH₃ | CH₃CH₂ |
| E-403 | COPh | n-C₄H₉ | CH₃ | CH₃CH₂ |
| E-404 | H | i-C₃H₇ | CH₃ | CH₃CH₂ |
| E-405 | CH₃ | i-C₃H₇ | CH₃ | CH₃CH₂ |
| E-406 | CH₂CH₃ | i-C₃H₇ | CH₃ | CH₃CH₂ |
| E-407 | CH₂CH═CH₂ | i-C₃H₇ | CH₃ | CH₃CH₂ |
| E-408 | CH₂Ph | i-C₃H₇ | CH₃ | CH₃CH₂ |
| E-409 | Ph | i-C₃H₇ | CH₃ | CH₃CH₂ |
| E-410 | COCH₃ | i-C₃H₇ | CH₃ | CH₃CH₂ |
| E-411 | COPh | i-C₃H₇ | CH₃ | CH₃CH₂ |
| E-412 | H | CH₂Ph | CH₃ | CH₃CH₂ |
| E-413 | CH₃ | CH₂Ph | CH₃ | CH₃CH₂ |
| E-414 | CH₂CH₃ | CH₂Ph | CH₃ | CH₃CH₂ |
| E-415 | CH₂CH═CH₂ | CH₂Ph | CH₃ | CH₃CH₂ |
| E-416 | CH₂Ph | CH₂Ph | CH₃ | CH₃CH₂ |
| E-417 | Ph | CH₂Ph | CH₃ | CH₃CH₂ |
| E-418 | COCH₃ | CH₂Ph | CH₃ | CH₃CH₂ |
| E-419 | COPh | CH₂Ph | CH₃ | CH₃CH₂ |
| E-420 | H | CH₂CH₂ | | CH₂CH₂ |
| E-421 | CH₃ | CH₂CH₂ | | CH₂CH₂ |
| E-422 | CH₂CH═CH₂ | CH₂CH₂ | | CH₂CH₂ |
| E-423 | CH₂Ph | CH₂CH₂ | | CH₂CH₂ |
| E-424 | Ph | CH₂CH₂ | | CH₂CH₂ |
| E-425 | COCH₃ | CH₂CH₂ | | CH₂CH₂ |
| E-426 | COPh | CH₂CH₂ | | CH₂CH₂ |
| E-427 | H | CH₂H₂CH₂ | | CH₂CH₂ |
| E-428 | CH₃ | CH₂H₂CH₂ | | CH₂CH₂ |
| E-429 | CH₂CH═CH₂ | CH₂H₂CH₂ | | CH₂CH₂ |
| E-430 | CH₂Ph | CH₂H₂CH₂ | | CH₂CH₂ |
| E-431 | Ph | CH₂H₂CH₂ | | CH₂CH₂ |
| E-432 | COCH₃ | CH₂H₂CH₂ | | CH₂CH₂ |
| E-433 | COPh | CH₂H₂CH₂ | | CH₂CH₂ |
| E-434 | H | CH₂CH₂CH₂CH₂ | | CH₂CH₂ |
| E-435 | CH₃ | CH₂CH₂CH₂CH₂ | | CH₂CH₂ |
| E-436 | CH₂CH═CH₂ | CH₂CH₂OH₂CH₂ | | CH₂CH₂ |
| E-437 | CH₂Ph | CH₂CH₂CH₂CH₂ | | CH₂CH₂ |
| E-438 | Ph | CH₂CH₂CH₂CH₂ | | CH₂CH₂ |
| E-439 | COCH₃ | CH₂CH₂CH₂CH₂ | | CH₂CH₂ |
| E-440 | COPh | CH₂CH₂CH₂CH₂ | | CH₂CH₂ |
| E-441 | H | CH₂CH₂CH₂CH₂CH₂ | | CH₂CH₂ |
| E-442 | CH₃ | CH₂CH₂CH₂CH₂CH₂ | | CH₂CH₂ |
| E-443 | CH₂CH═CH₂ | CH₂CH₂CH₂CH₂CH₂ | | CH₂CH₂ |
| E-444 | CH₂Ph | CH₂CH₂CH₂CH₂CH₂ | | CH₂CH₂ |
| E-445 | Ph | CH₂CH₂CH₂CH₂CH₂ | | CH₂CH₂ |
| E-446 | COCH₃ | CH₂CH₂CH₂CH₂CH₂ | | CH₂CH₂ |
| E-447 | COPh | CH₂CH₂CH₂CH₂CH₂ | | CH₂CH₂ |
| E-448 | H | H | H | CH₂CH═CH₂ |
| E-449 | CH₃ | H | H | CH₂CH═CH₂ |
| E-450 | CH₂CH₃ | H | H | CH₂CH═CH₂ |
| E-451 | CH₂CH═CH₂ | H | H | CH₂CH═CH₂ |
| E-452 | CH₂Ph | H | H | CH₂CH═CH₂ |
| E-453 | Ph | H | H | CH₂CH═CH₂ |
| E-454 | COCH₃ | H | H | CH₂CH═CH₂ |
| E-455 | COPh | H | H | CH₂CH═CH₂ |
| E-456 | H | CH₃ | H | CH₂CH═CH₂ |
| E-457 | CH₃ | CH₃ | H | CH₂CH═CH₂ |
| E-458 | CH₂CH₃ | CH₃ | H | CH₂CH═CH₂ |
| E-459 | CH₂CH═CH₂ | CH₃ | H | CH₂CH═CH₂ |
| E-460 | CH₂Ph | CH₃ | H | CH₂CH═CH₂ |
| E-461 | Ph | CH₃ | H | CH₂CH═CH₂ |
| E-462 | COCH₃ | CH₃ | H | CH₂CH═CH₂ |
| E-463 | COPh | CH₃ | H | CH₂CH═CH₂ |
| E-464 | H | CH₃CH₂ | H | CH₂CH═CH₂ |
| E-465 | CH₃ | CH₃CH₂ | H | CH₂CH═CH₂ |
| E-466 | CH₂CH₃ | CH₃CH₂ | H | CH₂CH═CH₂ |
| E-467 | CH₂CH═CH₂ | CH₃CH₂ | H | CH₂CH═CH₂ |
| E-468 | CH₂Ph | CH₃CH₂ | H | CH₂CH═CH₂ |
| E-469 | Ph | CH₃CH₂ | H | CH₂CH═CH₂ |
| E-470 | COCH₃ | CH₃CH₂ | H | CH₂CH═CH₂ |
| E-471 | COPh | n-C₃H₇ | H | CH₂CH═CH₂ |
| E-472 | H | n-C₃H₇ | H | CH₂CH═CH₂ |
| E-473 | CH₃ | n-C₃H₇ | H | CH₂CH═CH₂ |
| E-474 | CH₂CH₃ | n-C₃H₇ | H | CH₂CH═CH₂ |
| E-475 | CH₂CH═CH₂ | n-C₃H₇ | H | CH₂CH═CH₂ |
| E-476 | CH₂Ph | n-C₃H₇ | H | CH₂CH═CH₂ |
| E-477 | Ph | n-C₃H₇ | H | CH₂CH═CH₂ |
| E-478 | COCH₃ | n-C₃H₇ | H | CH₂CH═CH₂ |
| E-479 | COPh | n-C₄H₉ | H | CH₂CH═CH₂ |
| E-480 | H | i-C₃H₇ | H | CH₂CH═CH₂ |
| E-481 | CH₃ | i-C₃H₇ | H | CH₂CH═CH₂ |
| E-482 | CH₂CH₃ | i-C₃H₇ | H | CH₂CH═CH₂ |
| E-483 | CH₂CH═CH₂ | i-C₃H₇ | H | CH₂CH═CH₂ |
| E-484 | CH₂Ph | i-C₃H₇ | H | CH₂CH═CH₂ |
| E-485 | Ph | i-C₃H₇ | H | CH₂CH═CH₂ |
| E-486 | COCH₃ | i-C₃H₇ | H | CH₂CH═CH₂ |
| E-487 | COPh | i-C₃H₇ | H | CH₂CH═CH₂ |
| E-488 | H | CH₂Ph | H | CH₂CH═CH₂ |
| E-489 | CH₃ | CH₂Ph | H | CH₂CH═CH₂ |
| E-490 | CH₂CH₃ | CH₂Ph | H | CH₂CH═CH₂ |
| E-491 | CH₂CH═CH₂ | CH₂Ph | H | CH₂CH═CH₂ |
| E-492 | CH₂Ph | CH₂Ph | H | CH₂CH═CH₂ |
| E-493 | Ph | CH₂Ph | H | CH₂CH═CH₂ |
| E-494 | COCH₃ | CH₂Ph | H | CH₂CH═CH₂ |
| E-495 | COPh | CH₂Ph | H | CH₂CH═CH₂ |
| E-496 | H | CH₃ | CH₃ | CH₂CH═CH₂ |
| E-497 | CH₃ | CH₃ | CH₃ | CH₂CH═CH₂ |
| E-498 | CH₂CH₃ | CH₃ | CH₃ | CH₂CH═CH₂ |
| E-499 | CH₂CH═CH₂ | CH₃ | CH₃ | CH₂CH═CH₂ |
| E-500 | CH₂Ph | CH₃ | CH₃ | CH₂CH═CH₂ |
| E-501 | Ph | CH₃ | CH₃ | CH₂CH═CH₂ |
| E-502 | COCH₃ | CH₃ | CH₃ | CH₂CH═CH₂ |
| E-503 | COPh | CH₃ | CH₃ | CH₂CH═CH₂ |
| E-504 | H | CH₃CH₂ | CH₃ | CH₂CH═CH₂ |
| E-505 | CH₃ | CH₃CH₂ | CH₃ | CH₂CH═CH₂ |
| E-506 | CH₂CH₃ | CH₃CH₂ | CH₃ | CH₂CH═CH₂ |
| E-507 | CH₂CH═CH₂ | CH₃CH₂ | CH₃ | CH₂CH═CH₂ |
| E-508 | CH₂Ph | CH₃CH₂ | CH₃ | CH₂CH═CH₂ |
| E-509 | Ph | CH₃CH₂ | CH₃ | CH₂CH═CH₂ |
| E-510 | COCH₃ | CH₃CH₂ | CH₃ | CH₂CH═CH₂ |
| E-511 | COPh | n-C₃H₇ | CH₃ | CH₂CH═CH₂ |
| E-512 | H | n-C₃H₇ | CH₃ | CH₂CH═CH₂ |
| E-513 | CH₃ | n-C₃H₇ | CH₃ | CH₂CH═CH₂ |
| E-514 | CH₂CH₃ | n-C₃H₇ | CH₃ | CH₂CH═CH₂ |
| E-515 | CH₂CH═CH₂ | n-C₃H₇ | CH₃ | CH₂CH═CH₂ |
| E-516 | CH₂Ph | n-C₃H₇ | CH₃ | CH₂CH═CH₂ |
| E-517 | Ph | n-C₃H₇ | CH₃ | CH₂CH═CH₂ |
| E-518 | COCH₃ | n-C₃H₇ | CH₃ | CH₂CH═CH₂ |
| E-519 | COPh | n-C₄H₉ | CH₃ | CH₂CH═CH₂ |
| E-520 | H | i-C₃H₇ | CH₃ | CH₂CH═CH₂ |
| E-521 | CH₃ | i-C₃H₇ | CH₃ | CH₂CH═CH₂ |
| E-522 | CH₂CH₃ | i-C₃H₇ | CH₃ | CH₂CH═CH₂ |
| E-523 | CH₂CH═CH₂ | i-C₃H₇ | CH₃ | CH₂CH═CH₂ |
| E-524 | CH₂Ph | i-C₃H₇ | CH₃ | CH₂CH═CH₂ |
| E-525 | Ph | i-C₃H₇ | CH₃ | CH₂CH═CH₂ |
| E-526 | COCH₃ | i-C₃H₇ | CH₃ | CH₂CH═CH₂ |
| E-527 | COPh | i-C₃H₇ | CH₃ | CH₂CH═CH₂ |
| E-528 | H | CH₂Ph | CH₃ | CH₂CH═CH₂ |
| E-529 | CH₃ | CH₂Ph | CH₃ | CH₂CH═CH₂ |
| E-530 | CH₂CH₃ | CH₂Ph | CH₃ | CH₂CH═CH₂ |
| E-531 | CH₂CH═CH₂ | CH₂Ph | CH₃ | CH₂CH═CH₂ |
| E-532 | CH₂Ph | CH₂Ph | CH₃ | CH₂CH═CH₂ |
| E-533 | Ph | CH₂Ph | CH₃ | CH₂CH═CH₂ |
| E-534 | COCH₃ | CH₂Ph | CH₃ | CH₂CH═CH₂ |
| E-535 | COPh | CH₂Ph | CH₃ | CH₂CH═CH₂ |
| E-536 | H | CH₂CH₂ | | CH₂CH═CH₂ |
| E-537 | CH₃ | CH₂CH₂ | | CH₂CH═CH₂ |
| E-538 | CH₂CH═CH₂ | CH₂CH₂ | | CH₂CH═CH₂ |
| E-539 | CH₂Ph | CH₂CH₂ | | CH₂CH═CH₂ |
| E-540 | Ph | CH₂CH₂ | | CH₂CH═CH₂ |
| E-541 | COCH₃ | CH₂CH₂ | | CH₂CH═CH₂ |
| E-542 | COPh | CH₂CH₂ | | CH₂CH═CH₂ |
| E-543 | H | CH₂H₂CH₂ | | CH₂CH═CH₂ |
| E-544 | CH₃ | CH₂H₂CH₂ | | CH₂CH═CH₂ |
| E-545 | CH₂CH═CH₂ | CH₂H₂CH₂ | | CH₂CH═CH₂ |
| E-546 | CH₂Ph | CH₂H₂CH₂ | | CH₂CH═CH₂ |
| E-547 | Ph | CH₂H₂CH₂ | | CH₂CH═CH₂ |
| E-548 | COCH₃ | CH₂H₂CH₂ | | CH₂CH═CH₂ |
| E-549 | COPh | CH₂H₂CH₂ | | CH₂CH═CH₂ |
| E-550 | H | CH₂CH₂CH₂CH₂ | | CH₂CH═CH₂ |
| E-551 | CH₃ | CH₂CH₂CH₂CH₂ | | CH₂CH═CH₂ |
| E-552 | CH₂CH═CH₂ | CH₂CH₂CH₂CH₂ | | CH₂CH═CH₂ |
| E-553 | CH₂Ph | CH₂CH₂CH₂CH₂ | | CH₂CH═CH₂ |
| E-554 | Ph | CH₂CH₂CH₂CH₂ | | CH₂CH═CH₂ |
| E-555 | COCH₃ | CH₂CH₂CH₂CH₂ | | CH₂CH═CH₂ |
| E-556 | COPh | CH₂CH₂CH₂CH₂ | | CH₂CH═CH₂ |
| E-557 | H | CH₂CH₂CH₂CH₂CH₂ | | CH₂CH═CH₂ |
| E-558 | CH₃ | CH₂CH₂CH₂CH₂CH₂ | | CH₂CH═CH₂ |
| E-559 | CH₂CH═CH₂ | CH₂CH₂CH₂CH₂CH₂ | | CH₂CH═CH₂ |
| E-560 | CH₂Ph | CH₂CH₂CH₂CH₂CH₂ | | CH₂CH═CH₂ |
| E-561 | Ph | CH₂CH₂CH₂CH₂CH₂ | | CH₂CH═CH₂ |
| E-562 | COCH₃ | CH₂CH₂CH₂CH₂CH₂ | | CH₂CH═CH₂ |
| E-563 | COPh | CH₂CH₂CH₂CH₂CH₂ | | CH₂CH═CH₂ |
| E-564 | H | H | H | CH₂Ph |
| E-565 | CH₃ | H | H | CH₂Ph |
| E-566 | CH₂CH₃ | H | H | CH₂Ph |
| E-567 | CH₂CH═CH₂ | H | H | CH₂Ph |
| E-568 | CH₂Ph | H | H | CH₂Ph |
| E-569 | Ph | H | H | CH₂Ph |
| E-570 | COCH₃ | H | H | CH₂Ph |
| E-571 | COPh | H | H | CH₂Ph |
| E-572 | H | CH₃ | H | CH₂Ph |
| E-573 | CH₃ | CH₃ | H | CH₂Ph |
| E-574 | CH₂CH₃ | CH₃ | H | CH₂Ph |
| E-575 | CH₂CH═CH₂ | CH₃ | H | CH₂Ph |
| E-576 | CH₂Ph | CH₃ | H | CH₂Ph |
| E-577 | Ph | CH₃ | H | CH₂Ph |
| E-578 | COCH₃ | CH₃ | H | CH₂Ph |
| E-579 | COPh | CH₃ | H | CH₂Ph |
| E-580 | H | CH₃CH₂ | H | CH₂Ph |
| E-581 | CH₃ | CH₃CH₂ | H | CH₂Ph |
| E-582 | CH₂CH₃ | CH₃CH₂ | H | CH₂Ph |
| E-583 | CH₂CH═CH₂ | CH₃CH₂ | H | CH₂Ph |
| E-584 | CH₂Ph | CH₃CH₂ | H | CH₂Ph |
| E-585 | Ph | CH₃CH₂ | H | CH₂Ph |
| E-586 | COCH₃ | CH₃CH₂ | H | CH₂Ph |
| E-587 | COPh | n-C₃H₇ | H | CH₂Ph |
| E-588 | H | n-C₃H₇ | H | CH₂Ph |
| E-589 | CH₃ | n-C₃H₇ | H | CH₂Ph |
| E-590 | CH₂CH₃ | n-C₃H₇ | H | CH₂Ph |
| E-591 | CH₂CH═CH₂ | n-C₃H₇ | H | CH₂Ph |
| E-592 | CH₂Ph | n-C₃H₇ | H | CH₂Ph |
| E-593 | Ph | n-C₃H₇ | H | CH₂Ph |
| E-594 | COCH₃ | n-C₃H₇ | H | CH₂Ph |
| E-595 | COPh | n-C₄H₉ | H | CH₂Ph |
| E-596 | H | i-C₃H₇ | H | CH₂Ph |
| E-597 | CH₃ | i-C₃H₇ | H | CH₂Ph |
| E-598 | CH₂CH₃ | i-C₃H₇ | H | CH₂Ph |
| E-599 | CH₂CH═CH₂ | i-C₃H₇ | H | CH₂Ph |
| E-600 | CH₂Ph | i-C₃H₇ | H | CH₂Ph |
| E-601 | Ph | i-C₃H₇ | H | CH₂Ph |
| E-602 | COCH₃ | i-C₃H₇ | H | CH₂Ph |
| E-603 | COPh | i-C₃H₇ | H | CH₂Ph |
| E-604 | H | CH₂Ph | H | CH₂Ph |
| E-605 | CH₃ | CH₂Ph | H | CH₂Ph |
| E-606 | CH₂CH₃ | CH₂Ph | H | CH₂Ph |
| E-607 | CH₂CH═CH₂ | CH₂Ph | H | CH₂Ph |
| E-608 | CH₂Ph | CH₂Ph | H | CH₂Ph |
| E-609 | Ph | CH₂Ph | H | CH₂Ph |
| E-610 | COCH₃ | CH₂Ph | H | CH₂Ph |
| E-611 | COPh | CH₂Ph | H | CH₂Ph |
| E-612 | CH₂CH₃ | CH₃ | CH₃ | CH₂Ph |
| E-613 | CH₂CH═CH₂ | CH₃ | CH₃ | CH₂Ph |
| E-614 | CH₂Ph | CH₃ | CH₃ | CH₂Ph |
| E-615 | Ph | CH₃ | CH₃ | CH₂Ph |
| E-616 | COCH₃ | CH₃ | CH₃ | CH₂Ph |
| E-617 | COPh | CH₃ | CH₃ | CH₂Ph |
| E-618 | H | CH₃CH₂ | CH₃ | CH₂Ph |
| E-619 | CH₃ | CH₃CH₂ | CH₃ | CH₂Ph |
| E-620 | CH₂CH₃ | CH₃CH₂ | CH₃ | CH₂Ph |
| E-621 | CH₂CH═CH₂ | CH₃CH₂ | CH₃ | CH₂Ph |
| E-622 | CH₂Ph | CH₃CH₂ | CH₃ | CH₂Ph |
| E-623 | Ph | CH₃CH₂ | CH₃ | CH₂Ph |
| E-624 | COCH₃ | CH₃CH₂ | CH₃ | CH₂Ph |
| E-625 | COPh | n-C₃H₇ | CH₃ | CH₂Ph |
| E-626 | H | n-C₃H₇ | CH₃ | CH₂Ph |
| E-627 | CH₃ | n-C₃H₇ | CH₃ | CH₂Ph |
| E-628 | CH₂CH₃ | n-C₃H₇ | CH₃ | CH₂Ph |
| E-629 | CH₂CH═CH₂ | n-C₃H₇ | CH₃ | CH₂Ph |
| E-630 | CH₂Ph | n-C₃H₇ | CH₃ | CH₂Ph |
| E-631 | Ph | n-C₃H₇ | CH₃ | CH₂Ph |
| E-632 | COCH₃ | n-C₃H₇ | CH₃ | CH₂Ph |
| E-633 | COPh | n-C₄H₉ | CH₃ | CH₂Ph |
| E-634 | H | i-C₃H₇ | CH₃ | CH₂Ph |
| E-635 | CH₃ | i-C₃H₇ | CH₃ | CH₂Ph |
| E-636 | CH₂CH₃ | i-C₃H₇ | CH₃ | CH₂Ph |
| E-637 | CH₂CH═CH₂ | i-C₃H₇ | CH₃ | CH₂Ph |
| E-638 | CH₂Ph | i-C₃H₇ | CH₃ | CH₂Ph |
| E-639 | Ph | i-C₃H₇ | CH₃ | CH₂Ph |
| E-640 | COCH₃ | i-C₃H₇ | CH₃ | CH₂Ph |
| E-641 | COPh | i-C₃H₇ | CH₃ | CH₂Ph |
| E-642 | H | CH₂Ph | CH₃ | CH₂Ph |
| E-643 | CH₃ | CH₂Ph | CH₃ | CH₂Ph |
| E-644 | CH₂CH₃ | CH₂Ph | CH₃ | CH₂Ph |
| E-645 | CH₂CH═CH₂ | CH₂Ph | CH₃ | CH₂Ph |
| E-646 | CH₂Ph | CH₂Ph | CH₃ | CH₂Ph |
| E-647 | Ph | CH₂Ph | CH₃ | CH₂Ph |
| E-648 | COCH₃ | CH₂Ph | CH₃ | CH₂Ph |
| E-649 | COPh | CH₂Ph | CH₃ | CH₂Ph |
| E-650 | H | CH₂CH₂ | | CH₂Ph |
| E-651 | CH₃ | CH₂CH₂ | | CH₂Ph |
| E-652 | CH₂CH═CH₂ | CH₂CH₂ | | CH₂Ph |
| E-653 | CH₂Ph | CH₂CH₂ | | CH₂Ph |
| E-654 | Ph | CH₂CH₂ | | CH₂Ph |
| E-655 | COCH₃ | CH₂CH₂ | | CH₂Ph |
| E-656 | COPh | CH₂CH₂ | | CH₂Ph |
| E-657 | H | CH₂H₂CH₂ | | CH₂Ph |
| E-658 | CH₃ | CH₂H₂CH₂ | | CH₂Ph |
| E-659 | CH₂CH═CH₂ | CH₂H₂CH₂ | | CH₂Ph |
| E-660 | CH₂Ph | CH₂H₂CH₂ | | CH₂Ph |
| E-661 | Ph | CH₂H₂CH₂ | | CH₂Ph |
| E-662 | COCH₃ | CH₂H₂CH₂ | | CH₂Ph |
| E-663 | COPh | CH₂H₂CH₂ | | CH₂Ph |
| E-664 | H | CH₂CH₂CH₂CH₂ | | CH₂Ph |
| E-665 | CH₃ | CH₂CH₂CH₂CH₂ | | CH₂Ph |
| E-666 | CH₂CH═CH₂ | CH₂CH₂CH₂CH₂ | | CH₂Ph |
| E-667 | CH₂Ph | CH₂CH₂CH₂CH₂ | | CH₂Ph |
| E-668 | Ph | CH₂CH₂CH₂CH₂ | | CH₂Ph |
| E-669 | COCH₃ | CH₂CH₂CH₂CH₂ | | CH₂Ph |
| E-670 | COPh | CH₂CH₂CH₂CH₂ | | CH₂Ph |
| E-671 | H | CH₂CH₂CH₂CH₂CH₂ | | CH₂Ph |
| E-672 | CH₃ | CH₂CH₂CH₂CH₂CH₂ | | CH₂Ph |
| E-673 | CH₂CH═CH₂ | CH₂CH₂CH₂CH₂CH₂ | | CH₂Ph |
| E-674 | CH₂Ph | CH₂CH₂CH₂CH₂CH₂ | | CH₂Ph |
| E-675 | Ph | CH₂CH₂CH₂CH₂CH₂ | | CH₂Ph |
| E-676 | COCH₃ | CH₂CH₂CH₂CH₂CH₂ | | CH₂Ph |
| E-677 | COPh | CH₂CH₂CH₂CH₂CH₂ | | CH₂Ph |
| E-678 | H | H | H | C₂H₄O(C═O)CH₃ |
| E-679 | CH₃ | H | H | C₂H₄O(C═O)CH₃ |
| E-680 | CH₂CH₃ | H | H | C₂H₄O(C═O)CH₃ |
| E-681 | CH₂CH═CH₂ | H | H | C₂H₄O(C═O)CH₃ |
| E-682 | CH₂Ph | H | H | C₂H₄O(C═O)CH₃ |
| E-683 | Ph | H | H | C₂H₄O(C═O)CH₃ |
| E-684 | COCH₃ | H | H | C₂H₄O(C═O)CH₃ |
| E-685 | COPh | H | H | C₂H₄O(C═O)CH₃ |
| E-686 | H | CH₃ | H | C₂H₄O(C═O)CH₃ |
| E-687 | CH₃ | CH₃ | H | C₂H₄O(C═O)CH₃ |
| E-688 | CH₂CH₃ | CH₃ | H | C₂H₄O(C═O)CH₃ |
| E-689 | CH₂CH═CH₂ | CH₃ | H | C₂H₄O(C═O)CH₃ |
| E-690 | CH₂Ph | CH₃ | H | C₂H₄O(C═O)CH₃ |
| E-691 | Ph | CH₃ | H | C₂H₄O(C═O)CH₃ |
| E-692 | COCH₃ | CH₃ | H | C₂H₄O(C═O)CH₃ |
| E-693 | COPh | CH₃ | H | C₂H₄O(C═O)CH₃ |
| E-694 | H | CH₃ | CH₃ | C₂H₄O(C═O)CH₃ |
| E-695 | CH₃ | CH₃ | CH₃ | C₂H₄O(C═O)CH₃ |
| E-696 | CH₂CH₃ | CH₃ | CH₃ | C₂H₄O(C═O)CH₃ |
| E-697 | CH₂CH═CH₂ | CH₃ | CH₃ | C₂H₄O(C═O)CH₃ |
| E-698 | CH₂Ph | CH₃ | CH₃ | C₂H₄O(C═O)CH₃ |
| E-699 | Ph | CH₃ | CH₃ | C₂H₄O(C═O)CH₃ |
| E-700 | COCH₃ | CH₃ | CH₃ | C₂H₄O(C═O)CH₃ |
| E-701 | COPh | CH₃ | CH₃ | C₂H₄O(C═O)CH₃ |

| | | | |
|---|---|---|---|
| E-702 | H | CH₂CH₂ | C₂H₄O(C═O)CH₃ |
| E-703 | CH₃ | CH₂CH₂ | C₂H₄O(C═O)CH₃ |
| E-704 | H | CH₂H₂CH₂ | C₂H₄O(C═O)CH₃ |
| E-705 | CH₃ | CH₂H₂CH₂ | C₂H₄O(C═O)CH₃ |
| E-706 | H | CH₂CH₂CH₂CH₂ | C₂H₄O(C═O)CH₃ |
| E-707 | CH₃ | CH₂CH₂CH₂CH₂ | C₂H₄O(C═O)CH₃ |
| E-708 | H | CH₂CH₂CH₂CH₂CH₂ | C₂H₄O(C═O)CH₃ |
| E-709 | CH₃ | CH₂CH₂CH₂CH₂CH₂ | C₂H₄O(C═O)CH₃ |

TABLE 6
Compounds of formula (If):

| | |
|---|---|
| | (If) |
| 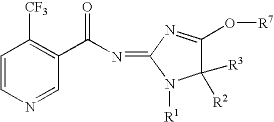 | |

| Compound | R¹ | R² | R³ | R⁷ |
|---|---|---|---|---|
| F-1 | H | H | H | CH₃ |
| F-2 | CH₃ | H | H | CH₃ |
| F-3 | CH₂CH₃ | H | H | CH₃ |
| F-4 | CH₂CH═CH₂ | H | H | CH₃ |
| F-5 | CH₂Ph | H | H | CH₃ |
| F-6 | Ph | H | H | CH₃ |
| F-7 | COCH₃ | H | H | CH₃ |
| F-8 | COPh | H | H | CH₃ |
| F-9 | H | CH₃ | H | CH₃ |
| F-10 | CH₃ | CH₃ | H | CH₃ |
| F-11 | CH₂CH₃ | CH₃ | H | CH₃ |
| F-12 | CH₂CH═CH₂ | CH₃ | H | CH₃ |
| F-13 | CH₂Ph | CH₃ | H | CH₃ |
| F-14 | Ph | CH₃ | H | CH₃ |
| F-15 | COCH₃ | CH₃ | H | CH₃ |
| F-16 | COPh | CH₃ | H | CH₃ |
| F-17 | H | CH₃CH₂ | H | CH₃ |
| F-18 | CH₃ | CH₃CH₂ | H | CH₃ |
| F-19 | CH₂CH₃ | CH₃CH₂ | H | CH₃ |
| F-20 | CH₂CH═CH₂ | CH₃CH₂ | H | CH₃ |
| F-21 | CH₂Ph | CH₃CH₂ | H | CH₃ |
| F-22 | Ph | CH₃CH₂ | H | CH₃ |
| F-23 | COCH₃ | CH₃CH₂ | H | CH₃ |
| F-24 | COPh | CH₃CH₂ | H | CH₃ |
| F-25 | H | n-C₃H₇ | H | CH₃ |
| F-26 | CH₃ | n-C₃H₇ | H | CH₃ |
| F-27 | CH₂CH₃ | n-C₃H₇ | H | CH₃ |
| F-28 | CH₂CH═CH₂ | n-C₃H₇ | H | CH₃ |
| F-29 | CH₂Ph | n-C₃H₇ | H | CH₃ |
| F-30 | Ph | n-C₃H₇ | H | CH₃ |
| F-31 | COCH₃ | n-C₃H₇ | H | CH₃ |
| F-32 | COPh | n-C₃H₇ | H | CH₃ |
| F-33 | H | i-C₃H₇ | H | CH₃ |
| F-34 | CH₃ | i-C₃H₇ | H | CH₃ |
| F-35 | CH₂CH₃ | i-C₃H₇ | H | CH₃ |
| F-36 | CH₂CH═CH₂ | i-C₃H₇ | H | CH₃ |
| F-37 | CH₂Ph | i-C₃H₇ | H | CH₃ |
| F-38 | Ph | i-C₃H₇ | H | CH₃ |
| F-39 | COCH₃ | i-C₃H₇ | H | CH₃ |
| F-40 | COPh | i-C₃H₇ | H | CH₃ |
| F-41 | H | n-C₄H₉ | H | CH₃ |
| F-42 | CH₃ | n-C₄H₉ | H | CH₃ |
| F-43 | CH₂CH₃ | n-C₄H₉ | H | CH₃ |
| F-44 | CH₂CH═CH₂ | n-C₄H₉ | H | CH₃ |
| F-45 | CH₂Ph | n-C₄H₉ | H | CH₃ |
| F-46 | Ph | n-C₄H₉ | H | CH₃ |
| F-47 | COCH₃ | n-C₄H₉ | H | CH₃ |
| F-48 | COPh | n-C₄H₉ | H | CH₃ |
| F-49 | H | i-C₄H₉ | H | CH₃ |
| F-50 | CH₃ | i-C₄H₉ | H | CH₃ |
| F-51 | CH₂CH₃ | i-C₄H₉ | H | CH₃ |
| F-52 | CH₂CH═CH₂ | i-C₄H₉ | H | CH₃ |
| F-53 | CH₂Ph | i-C₄H₉ | H | CH₃ |
| F-54 | Ph | i-C₄H₉ | H | CH₃ |
| F-55 | COCH₃ | i-C₄H₉ | H | CH₃ |
| F-56 | COPh | i-C₄H₉ | H | CH₃ |
| F-57 | H | s-C₄H₉ | H | CH₃ |
| F-58 | CH₃ | s-C₄H₉ | H | CH₃ |
| F-59 | CH₂CH₃ | s-C₄H₉ | H | CH₃ |
| F-60 | CH₂CH═CH₂ | s-C₄H₉ | H | CH₃ |
| F-61 | CH₂Ph | s-C₄H₉ | H | CH₃ |
| F-62 | Ph | s-C₄H₉ | H | CH₃ |
| F-63 | COCH₃ | s-C₄H₉ | H | CH₃ |
| F-64 | COPh | s-C₄H₉ | H | CH₃ |
| F-65 | H | t-C₄H₉ | H | CH₃ |
| F-66 | CH₃ | t-C₄H₉ | H | CH₃ |
| F-67 | CH₂CH₃ | t-C₄H₉ | H | CH₃ |
| F-68 | CH₂CH═CH₂ | t-C₄H₉ | H | CH₃ |
| F-69 | CH₂Ph | t-C₄H₉ | H | CH₃ |
| F-70 | Ph | t-C₄H₉ | H | CH₃ |
| F-71 | COCH₃ | t-C₄H₉ | H | CH₃ |
| F-72 | COPh | t-C₄H₉ | H | CH₃ |
| F-73 | H | CH₂Ph | H | CH₃ |
| F-74 | CH₃ | CH₂Ph | H | CH₃ |
| F-75 | CH₂CH₃ | CH₂Ph | H | CH₃ |
| F-76 | CH₂CH═CH₂ | CH₂Ph | H | CH₃ |
| F-77 | CH₂Ph | CH₂Ph | H | CH₃ |
| F-78 | Ph | CH₂Ph | H | CH₃ |
| F-79 | COCH₃ | CH₂Ph | H | CH₃ |
| F-80 | COPh | CH₂Ph | H | CH₃ |
| F-81 | H | Ph | H | CH₃ |
| F-82 | CH₃ | Ph | H | CH₃ |
| F-83 | CH₂CH₃ | Ph | H | CH₃ |
| F-84 | CH₂CH═CH₂ | Ph | H | CH₃ |
| F-85 | CH₂Ph | Ph | H | CH₃ |
| F-86 | Ph | Ph | H | CH₃ |
| F-87 | COCH₃ | Ph | H | CH₃ |
| F-88 | COPh | Ph | H | CH₃ |
| F-89 | H | CH₂OH | H | CH₃ |
| F-90 | CH₃ | CH₂OH | H | CH₃ |
| F-91 | CH₂CH₃ | CH₂OH | H | CH₃ |
| F-92 | CH₂CH═CH₂ | CH₂OH | H | CH₃ |
| F-93 | CH₂Ph | CH₂OH | H | CH₃ |
| F-94 | Ph | CH₂OH | H | CH₃ |
| F-95 | COCH₃ | CH₂OH | H | CH₃ |
| F-96 | COPh | CH₂OH | H | CH₃ |
| F-97 | H | CH(OH)CH₃ | H | CH₃ |
| F-98 | CH₃ | CH(OH)CH₃ | H | CH₃ |
| F-99 | CH₂CH₃ | CH(OH)CH₃ | H | CH₃ |
| F-100 | CH₂CH═CH₂ | CH(OH)CH₃ | H | CH₃ |
| F-101 | CH₂Ph | CH(OH)CH₃ | H | CH₃ |
| F-102 | Ph | CH(OH)CH₃ | H | CH₃ |
| F-103 | COCH₃ | CH(OH)CH₃ | H | CH₃ |
| F-104 | COPh | CH(OH)CH₃ | H | CH₃ |
| F-105 | H | CH₂SH | H | CH₃ |
| F-106 | CH₃ | CH₂SH | H | CH₃ |
| F-107 | CH₂CH₃ | CH₂SH | H | CH₃ |
| F-108 | CH₂CH═CH₂ | CH₂SH | H | CH₃ |
| F-109 | CH₂Ph | CH₂SH | H | CH₃ |
| F-110 | Ph | CH₂SH | H | CH₃ |
| F-111 | COCH₃ | CH₂SH | H | CH₃ |
| F-112 | COPh | CH₂SH | H | CH₃ |
| F-113 | H | CH₂SCH₂CH₃ | H | CH₃ |
| F-114 | CH₃ | CH₂SCH₂CH₃ | H | CH₃ |
| F-115 | CH₂CH₃ | CH₂SCH₂CH₃ | H | CH₃ |
| F-116 | CH₂CH═CH₂ | CH₂SCH₂CH₃ | H | CH₃ |
| F-117 | CH₂Ph | CH₂SCH₂CH₃ | H | CH₃ |
| F-118 | Ph | CH₂SCH₂CH₃ | H | CH₃ |
| F-119 | COCH₃ | CH₂SCH₂CH₃ | H | CH₃ |
| F-120 | COPh | CH₂SCH₂CH₃ | H | CH₃ |
| F-121 | H | CH₂CONH₂ | H | CH₃ |
| F-122 | CH₃ | CH₂CONH₂ | H | CH₃ |
| F-123 | CH₂CH₃ | CH₂CONH₂ | H | CH₃ |
| F-124 | CH₂CH═CH₂ | CH₂CONH₂ | H | CH₃ |
| F-125 | CH₂Ph | CH₂CONH₂ | H | CH₃ |
| F-126 | Ph | CH₂CONH₂ | H | CH₃ |
| F-127 | COCH₃ | CH₂CONH₂ | H | CH₃ |
| F-128 | COPh | CH₂CONH₂ | H | CH₃ |
| F-129 | H | CH₃ | CH₃ | CH₃ |
| F-130 | CH₃ | CH₃ | CH₃ | CH₃ |
| F-131 | CH₂CH₃ | CH₃ | CH₃ | CH₃ |
| F-132 | CH₂CH═CH₂ | CH₃ | CH₃ | CH₃ |
| F-133 | CH₂Ph | CH₃ | CH₃ | CH₃ |
| F-134 | Ph | CH₃ | CH₃ | CH₃ |
| F-135 | COCH₃ | CH₃ | CH₃ | CH₃ |
| F-136 | COPh | CH₃ | CH₃ | CH₃ |
| F-137 | H | CH₃CH₂ | CH₃ | CH₃ |
| F-138 | CH₃ | CH₃CH₂ | CH₃ | CH₃ |
| F-139 | CH₂CH₃ | CH₃CH₂ | CH₃ | CH₃ |
| F-140 | CH₂CH═CH₂ | CH₃CH₂ | CH₃ | CH₃ |
| F-141 | CH₂Ph | CH₃CH₂ | CH₃ | CH₃ |
| F-142 | Ph | CH₃CH₂ | CH₃ | CH₃ |
| F-143 | COCH₃ | CH₃CH₂ | CH₃ | CH₃ |
| F-144 | COPh | CH₃CH₂ | CH₃ | CH₃ |
| F-145 | H | n-C₃H₇ | CH₃ | CH₃ |
| F-146 | CH₃ | n-C₃H₇ | CH₃ | CH₃ |
| F-147 | CH₂CH₃ | n-C₃H₇ | CH₃ | CH₃ |
| F-148 | CH₂CH═CH₂ | n-C₃H₇ | CH₃ | CH₃ |
| F-149 | CH₂Ph | n-C₃H₇ | CH₃ | CH₃ |
| F-150 | Ph | n-C₃H₇ | CH₃ | CH₃ |
| F-151 | COCH₃ | n-C₃H₇ | CH₃ | CH₃ |
| F-152 | COPh | n-C₃H₇ | CH₃ | CH₃ |
| F-153 | H | i-C₃H₇ | CH₃ | CH₃ |
| F-154 | CH₃ | i-C₃H₇ | CH₃ | CH₃ |
| F-155 | CH₂CH₃ | i-C₃H₇ | CH₃ | CH₃ |
| F-156 | CH₂CH═CH₂ | i-C₃H₇ | CH₃ | CH₃ |
| F-157 | CH₂Ph | i-C₃H₇ | CH₃ | CH₃ |
| F-158 | Ph | i-C₃H₇ | CH₃ | CH₃ |
| F-159 | COCH₃ | i-C₃H₇ | CH₃ | CH₃ |
| F-160 | COPh | i-C₃H₇ | CH₃ | CH₃ |
| F-161 | H | n-C₄H₉ | CH₃ | CH₃ |
| F-162 | CH₃ | n-C₄H₉ | CH₃ | CH₃ |
| F-163 | CH₂CH₃ | n-C₄H₉ | CH₃ | CH₃ |
| F-164 | CH₂CH═CH₂ | n-C₄H₉ | CH₃ | CH₃ |
| F-165 | CH₂Ph | n-C₄H₉ | CH₃ | CH₃ |
| F-166 | Ph | n-C₄H₉ | CH₃ | CH₃ |
| F-167 | COCH₃ | n-C₄H₉ | CH₃ | CH₃ |
| F-168 | COPh | n-C₄H₉ | CH₃ | CH₃ |
| F-169 | H | i-C₄H₉ | CH₃ | CH₃ |
| F-170 | CH₃ | i-C₄H₉ | CH₃ | CH₃ |
| F-171 | CH₂CH₃ | i-C₄H₉ | CH₃ | CH₃ |
| F-172 | CH₂CH═CH₂ | i-C₄H₉ | CH₃ | CH₃ |
| F-173 | CH₂Ph | i-C₄H₉ | CH₃ | CH₃ |
| F-174 | Ph | i-C₄H₉ | CH₃ | CH₃ |
| F-175 | COCH₃ | i-C₄H₉ | CH₃ | CH₃ |
| F-176 | COPh | i-C₄H₉ | CH₃ | CH₃ |
| F-177 | H | s-C₄H₉ | CH₃ | CH₃ |
| F-178 | CH₃ | s-C₄H₉ | CH₃ | CH₃ |
| F-179 | CH₂CH₃ | s-C₄H₉ | CH₃ | CH₃ |
| F-180 | CH₂CH═CH₂ | s-C₄H₉ | CH₃ | CH₃ |
| F-181 | CH₂Ph | s-C₄H₉ | CH₃ | CH₃ |
| F-182 | Ph | s-C₄H₉ | CH₃ | CH₃ |
| F-183 | COCH₃ | s-C₄H₉ | CH₃ | CH₃ |
| F-184 | COPh | s-C₄H₉ | CH₃ | CH₃ |
| F-185 | H | t-C₄H₉ | CH₃ | CH₃ |
| F-186 | CH₃ | t-C₄H₉ | CH₃ | CH₃ |
| F-187 | CH₂CH₃ | t-C₄H₉ | CH₃ | CH₃ |
| F-188 | CH₂CH═CH₂ | t-C₄H₉ | CH₃ | CH₃ |
| F-189 | CH₂Ph | t-C₄H₉ | CH₃ | CH₃ |
| F-190 | Ph | t-C₄H₉ | CH₃ | CH₃ |
| F-191 | COCH₃ | t-C₄H₉ | CH₃ | CH₃ |
| F-192 | COPh | t-C₄H₉ | CH₃ | CH₃ |
| F-193 | H | CH₂Ph | CH₃ | CH₃ |
| F-194 | CH₃ | CH₂Ph | CH₃ | CH₃ |
| F-195 | CH₂CH₃ | CH₂Ph | CH₃ | CH₃ |
| F-196 | CH₂CH═CH₂ | CH₂Ph | CH₃ | CH₃ |
| F-197 | CH₂Ph | CH₂Ph | CH₃ | CH₃ |
| F-198 | Ph | CH₂Ph | CH₃ | CH₃ |
| F-199 | COCH₃ | CH₂Ph | CH₃ | CH₃ |
| F-200 | COPh | CH₂Ph | CH₃ | CH₃ |
| F-201 | H | Ph | CH₃ | CH₃ |
| F-202 | CH₃ | Ph | CH₃ | CH₃ |
| F-203 | CH₂CH₃ | Ph | CH₃ | CH₃ |
| F-204 | CH₂CH═CH₂ | Ph | CH₃ | CH₃ |
| F-205 | CH₂Ph | Ph | CH₃ | CH₃ |
| F-206 | Ph | Ph | CH₃ | CH₃ |
| F-207 | COCH₃ | Ph | CH₃ | CH₃ |
| F-208 | COPh | Ph | CH₃ | CH₃ |
| F-209 | H | CH₂OH | CH₃ | CH₃ |
| F-210 | CH₃ | CH₂OH | CH₃ | CH₃ |
| F-211 | CH₂CH₃ | CH₂OH | CH₃ | CH₃ |
| F-212 | CH₂CH═CH₂ | CH₂OH | CH₃ | CH₃ |
| F-213 | CH₂Ph | CH₂OH | CH₃ | CH₃ |
| F-214 | Ph | CH₂OH | CH₃ | CH₃ |
| F-215 | COCH₃ | CH₂OH | CH₃ | CH₃ |
| F-216 | COPh | CH₂OH | CH₃ | CH₃ |
| F-217 | H | CH(OH)CH₃ | CH₃ | CH₃ |
| F-218 | CH₃ | CH(OH)CH₃ | CH₃ | CH₃ |
| F-219 | CH₂CH₃ | CH(OH)CH₃ | CH₃ | CH₃ |
| F-220 | CH₂CH═CH₂ | CH(OH)CH₃ | CH₃ | CH₃ |
| F-221 | CH₂Ph | CH(OH)CH₃ | CH₃ | CH₃ |
| F-222 | Ph | CH(OH)CH₃ | CH₃ | CH₃ |
| F-223 | COCH₃ | CH(OH)CH₃ | CH₃ | CH₃ |
| F-224 | COPh | CH(OH)CH₃ | CH₃ | CH₃ |
| F-225 | H | CH₂SH | CH₃ | CH₃ |
| F-226 | CH₃ | CH₂SH | CH₃ | CH₃ |
| F-227 | CH₂CH₃ | CH₂SH | CH₃ | CH₃ |
| F-228 | CH₂CH═CH₂ | CH₂SH | CH₃ | CH₃ |
| F-229 | CH₂Ph | CH₂SH | CH₃ | CH₃ |
| F-230 | Ph | CH₂SH | CH₃ | CH₃ |
| F-231 | COCH₃ | CH₂SH | CH₃ | CH₃ |
| F-232 | COPh | CH₂SH | CH₃ | CH₃ |
| F-233 | H | CH₂SCH₂CH₃ | CH₃ | CH₃ |
| F-234 | CH₃ | CH₂SCH₂CH₃ | CH₃ | CH₃ |
| F-235 | CH₂CH₃ | CH₂SCH₂CH₃ | CH₃ | CH₃ |
| F-236 | CH₂CH═CH₂ | CH₂SCH₂CH₃ | CH₃ | CH₃ |
| F-237 | CH₂Ph | CH₂SCH₂CH₃ | CH₃ | CH₃ |
| F-238 | Ph | CH₂SCH₂CH₃ | CH₃ | CH₃ |
| F-239 | COCH₃ | CH₂SCH₂CH₃ | CH₃ | CH₃ |
| F-240 | COPh | CH₂SCH₂CH₃ | CH₃ | CH₃ |
| F-241 | H | CH₂CONH₂ | CH₃ | CH₃ |
| F-242 | CH₃ | CH₂CONH₂ | CH₃ | CH₃ |
| F-243 | CH₂CH₃ | CH₂CONH₂ | CH₃ | CH₃ |
| F-244 | CH₂CH═CH₂ | CH₂CONH₂ | CH₃ | CH₃ |
| F-245 | CH₂Ph | CH₂CONH₂ | CH₃ | CH₃ |
| F-246 | Ph | CH₂CONH₂ | CH₃ | CH₃ |
| F-247 | COCH₃ | CH₂CONH₂ | CH₃ | CH₃ |
| F-248 | COPh | CH₂CONH₂ | CH₃ | CH₃ |
| F-249 | H | CH₂CH₂CONH₂ | CH₃ | CH₃ |
| F-250 | CH₃ | CH₂CH₂CONH₂ | CH₃ | CH₃ |
| F-251 | CH₂CH₃ | CH₂CH₂CONH₂ | CH₃ | CH₃ |
| F-252 | CH₂CH═CH₂ | CH₂CH₂CONH₂ | CH₃ | CH₃ |
| F-253 | CH₂Ph | CH₂CH₂CONH₂ | CH₃ | CH₃ |
| F-254 | Ph | CH₂CH₂CONH₂ | CH₃ | CH₃ |
| F-255 | COCH₃ | CH₂CH₂CONH₂ | CH₃ | CH₃ |
| F-256 | COPh | CH₂CH₂CONH₂ | CH₃ | CH₃ |
| F-257 | CH₃ | CH₃CH₂ | CH₃CH₂ | CH₃ |
| F-258 | CH₂CH₃ | CH₃CH₂ | CH₃CH₂ | CH₃ |
| F-259 | CH₂CH═CH₂ | CH₃CH₂ | CH₃CH₂ | CH₃ |
| F-260 | CH₂Ph | CH₃CH₂ | CH₃CH₂ | CH₃ |
| F-261 | Ph | CH₃CH₂ | CH₃CH₂ | CH₃ |
| F-262 | COCH₃ | CH₃CH₂ | CH₃CH₂ | CH₃ |
| F-263 | COPh | CH₃CH₂ | CH₃CH₂ | CH₃ |
| F-264 | H | n-C₃H₇ | CH₃CH₂ | CH₃ |
| F-265 | CH₃ | n-C₃H₇ | CH₃CH₂ | CH₃ |
| F-266 | CH₂CH₃ | n-C₃H₇ | CH₃CH₂ | CH₃ |
| F-267 | CH₂CH═CH₂ | n-C₃H₇ | CH₃CH₂ | CH₃ |
| F-268 | CH₂Ph | n-C₃H₇ | CH₃CH₂ | CH₃ |
| F-269 | Ph | n-C₃H₇ | CH₃CH₂ | CH₃ |
| F-270 | COCH₃ | n-C₃H₇ | CH₃CH₂ | CH₃ |
| F-271 | COPh | n-C₃H₇ | CH₃CH₂ | CH₃ |
| F-272 | H | i-C₃H₇ | CH₃CH₂ | CH₃ |
| F-273 | CH₃ | i-C₃H₇ | CH₃CH₂ | CH₃ |
| F-274 | CH₂CH₃ | i-C₃H₇ | CH₃CH₂ | CH₃ |
| F-275 | CH₂CH═CH₂ | i-C₃H₇ | CH₃CH₂ | CH₃ |
| F-276 | CH₂Ph | i-C₃H₇ | CH₃CH₂ | CH₃ |
| F-277 | Ph | i-C₃H₇ | CH₃CH₂ | CH₃ |
| F-278 | COCH₃ | i-C₃H₇ | CH₃CH₂ | CH₃ |
| F-279 | COPh | i-C₃H₇ | CH₃CH₂ | CH₃ |
| F-280 | H | n-C₄H₉ | CH₃CH₂ | CH₃ |
| F-281 | CH₃ | n-C₄H₉ | CH₃CH₂ | CH₃ |
| F-282 | CH₂CH₃ | n-C₄H₉ | CH₃CH₂ | CH₃ |
| F-283 | CH₂CH═CH₂ | n-C₄H₉ | CH₃CH₂ | CH₃ |
| F-284 | CH₂Ph | n-C₄H₉ | CH₃CH₂ | CH₃ |
| F-285 | Ph | n-C₄H₉ | CH₃CH₂ | CH₃ |
| F-286 | COCH₃ | n-C₄H₉ | CH₃CH₂ | CH₃ |
| F-287 | COPh | n-C₄H₉ | CH₃CH₂ | CH₃ |
| F-288 | H | t-C₄H₉ | CH₃CH₂ | CH₃ |
| F-289 | CH₃ | t-C₄H₉ | CH₃CH₂ | CH₃ |
| F-290 | CH₂CH₃ | t-C₄H₉ | CH₃CH₂ | CH₃ |
| F-291 | CH₂CH═CH₂ | t-C₄H₉ | CH₃CH₂ | CH₃ |
| F-292 | CH₂Ph | t-C₄H₉ | CH₃CH₂ | CH₃ |
| F-293 | Ph | t-C₄H₉ | CH₃CH₂ | CH₃ |
| F-294 | COCH₃ | t-C₄H₉ | CH₃CH₂ | CH₃ |
| F-295 | COPh | t-C₄H₉ | CH₃CH₂ | CH₃ |
| F-296 | H | CH₂Ph | CH₃CH₂ | CH₃ |
| F-297 | CH₃ | CH₂Ph | CH₃CH₂ | CH₃ |
| F-298 | CH₂CH₃ | CH₂Ph | CH₃CH₂ | CH₃ |
| F-299 | CH₂CH═CH₂ | CH₂Ph | CH₃CH₂ | CH₃ |
| F-300 | CH₂Ph | CH₂Ph | CH₃CH₂ | CH₃ |
| F-301 | Ph | CH₂Ph | CH₃CH₂ | CH₃ |
| F-302 | COCH₃ | CH₂Ph | CH₃CH₂ | CH₃ |
| F-303 | COPh | CH₂Ph | CH₃CH₂ | CH₃ |
| F-304 | H | CH₂CH₂ | | CH₃ |
| F-305 | CH₃ | CH₂CH₂ | | CH₃ |
| F-306 | CH₂CH═CH₂ | CH₂CH₂ | | CH₃ |
| F-307 | CH₂Ph | CH₂CH₂ | | CH₃ |
| F-308 | Ph | CH₂CH₂ | | CH₃ |
| F-309 | COCH₃ | CH₂CH₂ | | CH₃ |
| F-310 | COPh | CH₂CH₂ | | CH₃ |
| F-311 | H | CH₂H₂CH₂ | | CH₃ |
| F-312 | CH₃ | CH₂H₂CH₂ | | CH₃ |
| F-313 | CH₂CH═CH₂ | CH₂H₂CH₂ | | CH₃ |
| F-314 | CH₂Ph | CH₂H₂CH₂ | | CH₃ |
| F-315 | Ph | CH₂H₂CH₂ | | CH₃ |
| F-316 | COCH₃ | CH₂H₂CH₂ | | CH₃ |
| F-317 | COPh | CH₂H₂CH₂ | | CH₃ |
| F-318 | H | CH₂CH₂CH₂CH₂ | | CH₃ |
| F-319 | CH₃ | CH₂CH₂CH₂CH₂ | | CH₃ |
| F-320 | CH₂CH═CH₂ | CH₂CH₂CH₂CH₂ | | CH₃ |
| F-321 | CH₂Ph | CH₂CH₂CH₂CH₂ | | CH₃ |
| F-322 | Ph | CH₂CH₂CH₂CH₂ | | CH₃ |
| F-323 | COCH₃ | CH₂CH₂CH₂CH₂ | | CH₃ |
| F-324 | COPh | CH₂CH₂CH₂CH₂ | | CH₃ |
| F-325 | H | CH₂CH₂CH₂CH₂CH₂ | | CH₃ |
| F-326 | CH₃ | CH₂CH₂CH₂CH₂CH₂ | | CH₃ |
| F-327 | CH₂CH═CH₂ | CH₂CH₂CH₂CH₂CH₂ | | CH₃ |
| F-328 | CH₂Ph | CH₂CH₂CH₂CH₂CH₂ | | CH₃ |
| F-329 | Ph | CH₂CH₂CH₂CH₂CH₂ | | CH₃ |
| F-330 | COCH₃ | CH₂CH₂CH₂CH₂CH₂ | | CH₃ |
| F-331 | COPh | CH₂CH₂CH₂CH₂CH₂ | | CH₃ |
| F-332 | H | H | H | CH₃CH₂ |
| F-333 | CH₃ | H | H | CH₃CH₂ |
| F-334 | CH₂CH₃ | H | H | CH₃CH₂ |
| F-335 | CH₂CH═CH₂ | H | H | CH₃CH₂ |
| F-336 | CH₂Ph | H | H | CH₃CH₂ |
| F-337 | Ph | H | H | CH₃CH₂ |
| F-338 | COCH₃ | H | H | CH₃CH₂ |
| F-339 | COPh | H | H | CH₃CH₂ |
| F-340 | H | CH₃ | H | CH₃CH₂ |
| F-341 | CH₃ | CH₃ | H | CH₃CH₂ |
| F-342 | CH₂CH₃ | CH₃ | H | CH₃CH₂ |
| F-343 | CH₂CH═CH₂ | CH₃ | H | CH₃CH₂ |
| F-344 | CH₂Ph | CH₃ | H | CH₃CH₂ |
| F-345 | Ph | CH₃ | H | CH₃CH₂ |
| F-346 | COCH₃ | CH₃ | H | CH₃CH₂ |
| F-347 | COPh | CH₃ | H | CH₃CH₂ |
| F-348 | H | CH₃CH₂ | H | CH₃CH₂ |
| F-349 | CH₃ | CH₃CH₂ | H | CH₃CH₂ |
| F-350 | CH₂CH₃ | CH₃CH₂ | H. | CH₃CH₂ |
| F-351 | CH₂CH═CH₂ | CH₃CH₂ | H | CH₃CH₂ |
| F-352 | CH₂Ph | CH₃CH₂ | H | CH₃CH₂ |
| F-353 | Ph | CH₃CH₂ | H | CH₃CH₂ |
| F-354 | COCH₃ | CH₃CH₂ | H | CH₃CH₂ |
| F-355 | COPh | n-C₃H₇ | H | CH₃CH₂ |
| F-356 | H | n-C₃H₇ | H | CH₃CH₂ |
| F-357 | CH₃ | n-C₃H₇ | H | CH₃CH₂ |
| F-358 | CH₂CH₃ | n-C₃H₇ | H | CH₃CH₂ |
| F-359 | CH₂CH═CH₂ | n-C₃H₇ | H | CH₃CH₂ |
| F-360 | CH₂Ph | n-C₃H₇ | H | CH₃CH₂ |
| F-361 | Ph | n-C₃H₇ | H | CH₃CH₂ |
| F-362 | COCH₃ | n-C₃H₇ | H | CH₃CH₂ |
| F-363 | COPh | n-C₄H₉ | H | CH₃CH₂ |
| F-364 | H | i-C₃H₇ | H | CH₃CH₂ |
| F-365 | CH₃ | i-C₃H₇ | H | CH₃CH₂ |
| F-366 | CH₂CH₃ | i-C₃H₇ | H | CH₃CH₂ |
| F-367 | CH₂CH═CH₂ | i-C₃H₇ | H | CH₃CH₂ |
| F-368 | CH₂Ph | i-C₃H₇ | H | CH₃CH₂ |
| F-369 | Ph | i-C₃H₇ | H | CH₃CH₂ |
| F-370 | COCH₃ | i-C₃H₇ | H | CH₃CH₂ |
| F-371 | COPh | i-C₃H₇ | H | CH₃CH₂ |
| F-372 | H | CH₂Ph | H | CH₃CH₂ |
| F-373 | CH₃ | CH₂Ph | H | CH₃CH₂ |
| F-374 | CH₂CH₃ | CH₂Ph | H | CH₃CH₂ |
| F-375 | CH₂CH═CH₂ | CH₂Ph | H | CH₃CH₂ |
| F-376 | CH₂Ph | CH₂Ph | H | CH₃CH₂ |
| F-377 | Ph | CH₂Ph | H | CH₃CH₂ |
| F-378 | COCH₃ | CH₂Ph | H | CH₃CH₂ |
| F-379 | COPh | CH₂Ph | H | CH₃CH₂ |
| F-380 | H | CH₃ | CH₃ | CH₃CH₂ |
| F-381 | CH₃ | CH₃ | CH₃ | CH₃CH₂ |
| F-382 | CH₂CH₃ | CH₃ | CH₃ | CH₃CH₂ |
| F-383 | CH₂CH═CH₂ | CH₃ | CH₃ | CH₃CH₂ |
| F-384 | CH₂Ph | CH₃ | CH₃ | CH₃CH₂ |
| F-385 | Ph | CH₃ | CH₃ | CH₃CH₂ |
| F-386 | COCH₃ | CH₃ | CH₃ | CH₃CH₂ |
| F-387 | COPh | CH₃ | CH₃ | CH₃CH₂ |
| F-388 | H | CH₃CH₂ | CH₃ | CH₃CH₂ |
| F-389 | CH₃ | CH₃CH₂ | CH₃ | CH₃CH₂ |
| F-390 | CH₂CH₃ | CH₃CH₂ | CH₃ | CH₃CH₂ |
| F-391 | CH₂CH═CH₂ | CH₃CH₂ | CH₃ | CH₃CH₂ |
| F-392 | CH₂Ph | CH₃CH₂ | CH₃ | CH₃CH₂ |
| F-393 | Ph | CH₃CH₂ | CH₃ | CH₃CH₂ |
| F-394 | COCH₃ | CH₃CH₂ | CH₃ | CH₃CH₂ |
| F-395 | COPh | n-C₃H₇ | CH₃ | CH₃CH₂ |
| F-396 | H | n-C₃H₇ | CH₃ | CH₃CH₂ |
| F-397 | CH₃ | n-C₃H₇ | CH₃ | CH₃CH₂ |
| F-398 | CH₂CH₃ | n-C₃H₇ | CH₃ | CH₃CH₂ |
| F-399 | CH₂CH═CH₂ | n-C₃H₇ | CH₃ | CH₃CH₂ |
| F-400 | CH₂Ph | n-C₃H₇ | CH₃ | CH₃CH₂ |
| F-401 | Ph | n-C₃H₇ | CH₃ | CH₃CH₂ |
| F-402 | COCH₃ | n-C₃H₇ | CH₃ | CH₃CH₂ |
| F-403 | COPh | n-C₄H₉ | CH₃ | CH₃CH₂ |
| F-404 | H | i-C₃H₇ | CH₃ | CH₃CH₂ |
| F-405 | CH₃ | i-C₃H₇ | CH₃ | CH₃CH₂ |
| F-406 | CH₂CH₃ | i-C₃H₇ | CH₃ | CH₃CH₂ |
| F-407 | CH₂CH═CH₂ | i-C₃H₇ | CH₃ | CH₃CH₂ |
| F-408 | CH₂Ph | i-C₃H₇ | CH₃ | CH₃CH₂ |
| F-409 | Ph | i-C₃H₇ | CH₃ | CH₃CH₂ |
| F-410 | COCH₃ | i-C₃H₇ | CH₃ | CH₃CH₂ |
| F-411 | COPh | i-C₃H₇ | CH₃ | CH₃CH₂ |
| F-412 | H | CH₂Ph | CH₃ | CH₃CH₂ |
| F-413 | CH₃ | CH₂Ph | CH₃ | CH₃CH₂ |
| F-414 | CH₂CH₃ | CH₂Ph | CH₃ | CH₃CH₂ |
| F-415 | CH₂CH═CH₂ | CH₂Ph | CH₃ | CH₃CH₂ |
| F-416 | CH₂Ph | CH₂Ph | CH₃ | CH₃CH₂ |
| F-417 | Ph | CH₂Ph | CH₃ | CH₃CH₂ |
| F-418 | COCH₃ | CH₂Ph | CH₃ | CH₃CH₂ |
| F-419 | COPh | CH₂Ph | CH₃ | CH₃CH₂ |
| F-420 | H | CH₂CH₂ | | CH₂CH₂ |
| F-421 | CH₃ | CH₂CH₂ | | CH₂CH₂ |
| F-422 | CH₂CH═CH₂ | CH₂CH₂ | | CH₂CH₂ |
| F-423 | CH₂Ph | CH₂CH₂ | | CH₂CH₂ |
| F-424 | Ph | CH₂CH₂ | | CH₂CH₂ |
| F-425 | COCH₃ | CH₂CH₂ | | CH₂CH₂ |
| F-426 | COPh | CH₂CH₂ | | CH₂CH₂ |
| F-427 | H | CH₂H₂CH₂ | | CH₂CH₂ |
| F-428 | CH₃ | CH₂H₂CH₂ | | CH₂CH₂ |
| F-429 | CH₂CH═CH₂ | CH₂H₂CH₂ | | CH₂CH₂ |
| F-430 | CH₂Ph | CH₂H₂CH₂ | | CH₂CH₂ |
| F-431 | Ph | CH₂H₂CH₂ | | CH₂CH₂ |
| F-432 | COCH₃ | CH₂H₂CH₂ | | CH₂CH₂ |
| F-433 | COPh | CH₂H₂CH₂ | | CH₂CH₂ |
| F-434 | H | CH₂CH₂CH₂CH₂ | | CH₂CH₂ |
| F-435 | CH₃ | CH₂CH₂CH₂CH₂ | | CH₂CH₂ |
| F-436 | CH₂CH═CH₂ | CH₂CH₂OH₂CH₂ | | CH₂CH₂ |
| F-437 | CH₂Ph | CH₂CH₂CH₂CH₂ | | CH₂CH₂ |
| F-438 | Ph | CH₂CH₂CH₂CH₂ | | CH₂CH₂ |
| F-439 | COCH₃ | CH₂CH₂CH₂CH₂ | | CH₂CH₂ |
| F-440 | COPh | CH₂CH₂CH₂CH₂ | | CH₂CH₂ |
| F-441 | H | CH₂CH₂CH₂CH₂CH₂ | | CH₂CH₂ |
| F-442 | CH₃ | CH₂CH₂CH₂CH₂CH₂ | | CH₂CH₂ |
| F-443 | CH₂CH═CH₂ | CH₂CH₂CH₂CH₂CH₂ | | CH₂CH₂ |
| F-444 | CH₂Ph | CH₂CH₂CH₂CH₂CH₂ | | CH₂CH₂ |
| F-445 | Ph | CH₂CH₂CH₂CH₂CH₂ | | CH₂CH₂ |
| F-446 | COCH₃ | CH₂CH₂CH₂CH₂CH₂ | | CH₂CH₂ |
| F-447 | COPh | CH₂CH₂CH₂CH₂CH₂ | | CH₂CH₂ |
| F-448 | H | H | H | CH₂CH═CH₂ |
| F-449 | CH₃ | H | H | CH₂CH═CH₂ |
| F-450 | CH₂CH₃ | H | H | CH₂CH═CH₂ |
| F-451 | CH₂CH═CH₂ | H | H | CH₂CH═CH₂ |
| F-452 | CH₂Ph | H | H | CH₂CH═CH₂ |
| F-453 | Ph | H | H | CH₂CH═CH₂ |
| F-454 | COCH₃ | H | H | CH₂CH═CH₂ |
| F-455 | COPh | H | H | CH₂CH═CH₂ |
| F-456 | H | CH₃ | H | CH₂CH═CH₂ |
| F-457 | CH₃ | CH₃ | H | CH₂CH═CH₂ |
| F-458 | CH₂CH₃ | CH₃ | H | CH₂CH═CH₂ |
| F-459 | CH₂CH═CH₂ | CH₃ | H | CH₂CH═CH₂ |
| F-460 | CH₂Ph | CH₃ | H | CH₂CH═CH₂ |
| F-461 | Ph | CH₃ | H | CH₂CH═CH₂ |
| F-462 | COCH₃ | CH₃ | H | CH₂CH═CH₂ |
| F-463 | COPh | CH₃ | H | CH₂CH═CH₂ |
| F-464 | H | CH₃CH₂ | H | CH₂CH═CH₂ |
| F-465 | CH₃ | CH₃CH₂ | H | CH₂CH═CH₂ |
| F-466 | CH₂CH₃ | CH₃CH₂ | H | CH₂CH═CH₂ |
| F-467 | CH₂CH═CH₂ | CH₃CH₂ | H | CH₂CH═CH₂ |
| F-468 | CH₂Ph | CH₃CH₂ | H | CH₂CH═CH₂ |
| F-469 | Ph | CH₃CH₂ | H | CH₂CH═CH₂ |
| F-470 | COCH₃ | CH₃CH₂ | H | CH₂CH═CH₂ |
| F-471 | COPh | n-C₃H₇ | H | CH₂CH═CH₂ |
| F-472 | H | n-C₃H₇ | H | CH₂CH═CH₂ |
| F-473 | CH₃ | n-C₃H₇ | H | CH₂CH═CH₂ |
| F-474 | CH₂CH₃ | n-C₃H₇ | H | CH₂CH═CH₂ |
| F-475 | CH₂CH═CH₂ | n-C₃H₇ | H | CH₂CH═CH₂ |
| F-476 | CH₂Ph | n-C₃H₇ | H | CH₂CH═CH₂ |
| F-477 | Ph | n-C₃H₇ | H | CH₂CH═CH₂ |
| F-478 | COCH₃ | n-C₃H₇ | H | CH₂CH═CH₂ |
| F-479 | COPh | n-C₄H₉ | H | CH₂CH═CH₂ |
| F-480 | H | i-C₃H₇ | H | CH₂CH═CH₂ |
| F-481 | CH₃ | i-C₃H₇ | H | CH₂CH═CH₂ |
| F-482 | CH₂CH₃ | i-C₃H₇ | H | CH₂CH═CH₂ |
| F-483 | CH₂CH═CH₂ | i-C₃H₇ | H | CH₂CH═CH₂ |
| F-484 | CH₂Ph | i-C₃H₇ | H | CH₂CH═CH₂ |
| F-485 | Ph | i-C₃H₇ | H | CH₂CH═CH₂ |
| F-486 | COCH₃ | i-C₃H₇ | H | CH₂CH═CH₂ |
| F-487 | COPh | i-C₃H₇ | H | CH₂CH═CH₂ |
| F-488 | H | CH₂Ph | H | CH₂CH═CH₂ |
| F-489 | CH₃ | CH₂Ph | H | CH₂CH═CH₂ |
| F-490 | CH₂CH₃ | CH₂Ph | H | CH₂CH═CH₂ |
| F-491 | CH₂CH═CH₂ | CH₂Ph | H | CH₂CH═CH₂ |
| F-492 | CH₂Ph | CH₂Ph | H | CH₂CH═CH₂ |
| F-493 | Ph | CH₂Ph | H | CH₂CH═CH₂ |
| F-494 | COCH₃ | CH₂Ph | H | CH₂CH═CH₂ |
| F-495 | COPh | CH₂Ph | H | CH₂CH═CH₂ |
| F-496 | H | CH₃ | CH₃ | CH₂CH═CH₂ |
| F-497 | CH₃ | CH₃ | CH₃ | CH₂CH═CH₂ |
| F-498 | CH₂CH₃ | CH₃ | CH₃ | CH₂CH═CH₂ |
| F-499 | CH₂CH═CH₂ | CH₃ | CH₃ | CH₂CH═CH₂ |
| F-500 | CH₂Ph | CH₃ | CH₃ | CH₂CH═CH₂ |
| F-501 | Ph | CH₃ | CH₃ | CH₂CH═CH₂ |
| F-502 | COCH₃ | CH₃ | CH₃ | CH₂CH═CH₂ |
| F-503 | COPh | CH₃ | CH₃ | CH₂CH═CH₂ |
| F-504 | H | CH₃CH₂ | CH₃ | CH₂CH═CH₂ |
| F-505 | CH₃ | CH₃CH₂ | CH₃ | CH₂CH═CH₂ |
| F-506 | CH₂CH₃ | CH₃CH₂ | CH₃ | CH₂CH═CH₂ |
| F-507 | CH₂CH═CH₂ | CH₃CH₂ | CH₃ | CH₂CH═CH₂ |
| F-508 | CH₂Ph | CH₃CH₂ | CH₃ | CH₂CH═CH₂ |
| F-509 | Ph | CH₃CH₂ | CH₃ | CH₂CH═CH₂ |
| F-510 | COCH₃ | CH₃CH₂ | CH₃ | CH₂CH═CH₂ |
| F-511 | COPh | n-C₃H₇ | CH₃ | CH₂CH═CH₂ |
| F-512 | H | n-C₃H₇ | CH₃ | CH₂CH═CH₂ |
| F-513 | CH₃ | n-C₃H₇ | CH₃ | CH₂CH═CH₂ |
| F-514 | CH₂CH₃ | n-C₃H₇ | CH₃ | CH₂CH═CH₂ |
| F-515 | CH₂CH═CH₂ | n-C₃H₇ | CH₃ | CH₂CH═CH₂ |
| F-516 | CH₂Ph | n-C₃H₇ | CH₃ | CH₂CH═CH₂ |
| F-517 | Ph | n-C₃H₇ | CH₃ | CH₂CH═CH₂ |
| F-518 | COCH₃ | n-C₃H₇ | CH₃ | CH₂CH═CH₂ |
| F-519 | COPh | n-C₄H₉ | CH₃ | CH₂CH═CH₂ |
| F-520 | H | i-C₃H₇ | CH₃ | CH₂CH═CH₂ |
| F-521 | CH₃ | i-C₃H₇ | CH₃ | CH₂CH═CH₂ |
| F-522 | CH₂CH₃ | i-C₃H₇ | CH₃ | CH₂CH═CH₂ |
| F-523 | CH₂CH═CH₂ | i-C₃H₇ | CH₃ | CH₂CH═CH₂ |
| F-524 | CH₂Ph | i-C₃H₇ | CH₃ | CH₂CH═CH₂ |
| F-525 | Ph | i-C₃H₇ | CH₃ | CH₂CH═CH₂ |
| F-526 | COCH₃ | i-C₃H₇ | CH₃ | CH₂CH═CH₂ |
| F-527 | COPh | i-C₃H₇ | CH₃ | CH₂CH═CH₂ |
| F-528 | H | CH₂Ph | CH₃ | CH₂CH═CH₂ |
| F-529 | CH₃ | CH₂Ph | CH₃ | CH₂CH═CH₂ |
| F-530 | CH₂CH₃ | CH₂Ph | CH₃ | CH₂CH═CH₂ |
| F-531 | CH₂CH═CH₂ | CH₂Ph | CH₃ | CH₂CH═CH₂ |
| F-532 | CH₂Ph | CH₂Ph | CH₃ | CH₂CH═CH₂ |
| F-533 | Ph | CH₂Ph | CH₃ | CH₂CH═CH₂ |
| F-534 | COCH₃ | CH₂Ph | CH₃ | CH₂CH═CH₂ |
| F-535 | COPh | CH₂Ph | CH₃ | CH₂CH═CH₂ |
| F-536 | H | CH₂CH₂ | | CH₂CH═CH₂ |
| F-537 | CH₃ | CH₂CH₂ | | CH₂CH═CH₂ |
| F-538 | CH₂CH═CH₂ | CH₂CH₂ | | CH₂CH═CH₂ |
| F-539 | CH₂Ph | CH₂CH₂ | | CH₂CH═CH₂ |
| F-540 | Ph | CH₂CH₂ | | CH₂CH═CH₂ |
| F-541 | COCH₃ | CH₂CH₂ | | CH₂CH═CH₂ |
| F-542 | COPh | CH₂CH₂ | | CH₂CH═CH₂ |
| F-543 | H | CH₂H₂CH₂ | | CH₂CH═CH₂ |
| F-544 | CH₃ | CH₂H₂CH₂ | | CH₂CH═CH₂ |
| F-545 | CH₂CH═CH₂ | CH₂H₂CH₂ | | CH₂CH═CH₂ |
| F-546 | CH₂Ph | CH₂H₂CH₂ | | CH₂CH═CH₂ |
| F-547 | Ph | CH₂H₂CH₂ | | CH₂CH═CH₂ |
| F-548 | COCH₃ | CH₂H₂CH₂ | | CH₂CH═CH₂ |
| F-549 | COPh | CH₂H₂CH₂ | | CH₂CH═CH₂ |
| F-550 | H | CH₂CH₂CH₂CH₂ | | CH₂CH═CH₂ |
| F-551 | CH₃ | CH₂CH₂CH₂CH₂ | | CH₂CH═CH₂ |
| F-552 | CH₂CH═CH₂ | CH₂CH₂CH₂CH₂ | | CH₂CH═CH₂ |
| F-553 | CH₂Ph | CH₂CH₂CH₂CH₂ | | CH₂CH═CH₂ |
| F-554 | Ph | CH₂CH₂CH₂CH₂ | | CH₂CH═CH₂ |
| F-555 | COCH₃ | CH₂CH₂CH₂CH₂ | | CH₂CH═CH₂ |
| F-556 | COPh | CH₂CH₂CH₂CH₂ | | CH₂CH═CH₂ |
| F-557 | H | CH₂CH₂CH₂CH₂CH₂ | | CH₂CH═CH₂ |
| F-558 | CH₃ | CH₂CH₂CH₂CH₂CH₂ | | CH₂CH═CH₂ |
| F-559 | CH₂CH═CH₂ | CH₂CH₂CH₂CH₂CH₂ | | CH₂CH═CH₂ |
| F-560 | CH₂Ph | CH₂CH₂CH₂CH₂CH₂ | | CH₂CH═CH₂ |
| F-561 | Ph | CH₂CH₂CH₂CH₂CH₂ | | CH₂CH═CH₂ |
| F-562 | COCH₃ | CH₂CH₂CH₂CH₂CH₂ | | CH₂CH═CH₂ |
| F-563 | COPh | CH₂CH₂CH₂CH₂CH₂ | | CH₂CH═CH₂ |
| F-564 | H | H | H | CH₂Ph |
| F-565 | CH₃ | H | H | CH₂Ph |
| F-566 | CH₂CH₃ | H | H | CH₂Ph |
| F-567 | CH₂CH═CH₂ | H | H | CH₂Ph |
| F-568 | CH₂Ph | H | H | CH₂Ph |
| F-569 | Ph | H | H | CH₂Ph |
| F-570 | COCH₃ | H | H | CH₂Ph |
| F-571 | COPh | H | H | CH₂Ph |
| F-572 | H | CH₃ | H | CH₂Ph |
| F-573 | CH₃ | CH₃ | H | CH₂Ph |
| F-574 | CH₂CH₃ | CH₃ | H | CH₂Ph |
| F-575 | CH₂CH═CH₂ | CH₃ | H | CH₂Ph |
| F-576 | CH₂Ph | CH₃ | H | CH₂Ph |
| F-577 | Ph | CH₃ | H | CH₂Ph |
| F-578 | COCH₃ | CH₃ | H | CH₂Ph |
| F-579 | COPh | CH₃ | H | CH₂Ph |
| F-580 | H | CH₃CH₂ | H | CH₂Ph |
| F-581 | CH₃ | CH₃CH₂ | H | CH₂Ph |
| F-582 | CH₂CH₃ | CH₃CH₂ | H | CH₂Ph |
| F-583 | CH₂CH═CH₂ | CH₃CH₂ | H | CH₂Ph |
| F-584 | CH₂Ph | CH₃CH₂ | H | CH₂Ph |
| F-585 | Ph | CH₃CH₂ | H | CH₂Ph |
| F-586 | COCH₃ | CH₃CH₂ | H | CH₂Ph |
| F-587 | COPh | n-C₃H₇ | H | CH₂Ph |
| F-588 | H | n-C₃H₇ | H | CH₂Ph |
| F-589 | CH₃ | n-C₃H₇ | H | CH₂Ph |
| F-590 | CH₂CH₃ | n-C₃H₇ | H | CH₂Ph |
| F-591 | CH₂CH═CH₂ | n-C₃H₇ | H | CH₂Ph |
| F-592 | CH₂Ph | n-C₃H₇ | H | CH₂Ph |
| F-593 | Ph | n-C₃H₇ | H | CH₂Ph |
| F-594 | COCH₃ | n-C₃H₇ | H | CH₂Ph |
| F-595 | COPh | n-C₄H₉ | H | CH₂Ph |
| F-596 | H | i-C₃H₇ | H | CH₂Ph |
| F-597 | CH₃ | i-C₃H₇ | H | CH₂Ph |
| F-598 | CH₂CH₃ | i-C₃H₇ | H | CH₂Ph |
| F-599 | CH₂CH═CH₂ | i-C₃H₇ | H | CH₂Ph |
| F-600 | CH₂Ph | i-C₃H₇ | H | CH₂Ph |
| F-601 | Ph | i-C₃H₇ | H | CH₂Ph |
| F-602 | COCH₃ | i-C₃H₇ | H | CH₂Ph |
| F-603 | COPh | i-C₃H₇ | H | CH₂Ph |
| F-604 | H | CH₂Ph | H | CH₂Ph |
| F-605 | CH₃ | CH₂Ph | H | CH₂Ph |
| F-606 | CH₂CH₃ | CH₂Ph | H | CH₂Ph |
| F-607 | CH₂CH═CH₂ | CH₂Ph | H | CH₂Ph |
| F-608 | CH₂Ph | CH₂Ph | H | CH₂Ph |
| F-609 | Ph | CH₂Ph | H | CH₂Ph |
| F-610 | COCH₃ | CH₂Ph | H | CH₂Ph |
| F-611 | COPh | CH₂Ph | H | CH₂Ph |
| F-612 | CH₂CH₃ | CH₃ | CH₃ | CH₂Ph |
| F-613 | CH₂CH═CH₂ | CH₃ | CH₃ | CH₂Ph |
| F-614 | CH₂Ph | CH₃ | CH₃ | CH₂Ph |
| F-615 | Ph | CH₃ | CH₃ | CH₂Ph |
| F-616 | COCH₃ | CH₃ | CH₃ | CH₂Ph |
| F-617 | COPh | CH₃ | CH₃ | CH₂Ph |
| F-618 | H | CH₃CH₂ | CH₃ | CH₂Ph |
| F-619 | CH₃ | CH₃CH₂ | CH₃ | CH₂Ph |
| F-620 | CH₂CH₃ | CH₃CH₂ | CH₃ | CH₂Ph |
| F-621 | CH₂CH═CH₂ | CH₃CH₂ | CH₃ | CH₂Ph |
| F-622 | CH₂Ph | CH₃CH₂ | CH₃ | CH₂Ph |
| F-623 | Ph | CH₃CH₂ | CH₃ | CH₂Ph |
| F-624 | COCH₃ | CH₃CH₂ | CH₃ | CH₂Ph |
| F-625 | COPh | n-C₃H₇ | CH₃ | CH₂Ph |
| F-626 | H | n-C₃H₇ | CH₃ | CH₂Ph |
| F-627 | CH₃ | n-C₃H₇ | CH₃ | CH₂Ph |
| F-628 | CH₂CH₃ | n-C₃H₇ | CH₃ | CH₂Ph |
| F-629 | CH₂CH═CH₂ | n-C₃H₇ | CH₃ | CH₂Ph |
| F-630 | CH₂Ph | n-C₃H₇ | CH₃ | CH₂Ph |
| F-631 | Ph | n-C₃H₇ | CH₃ | CH₂Ph |
| F-632 | COCH₃ | n-C₃H₇ | CH₃ | CH₂Ph |
| F-633 | COPh | n-C₄H₉ | CH₃ | CH₂Ph |
| F-634 | H | i-C₃H₇ | CH₃ | CH₂Ph |
| F-635 | CH₃ | i-C₃H₇ | CH₃ | CH₂Ph |
| F-636 | CH₂CH₃ | i-C₃H₇ | CH₃ | CH₂Ph |
| F-637 | CH₂CH═CH₂ | i-C₃H₇ | CH₃ | CH₂Ph |
| F-638 | CH₂Ph | i-C₃H₇ | CH₃ | CH₂Ph |
| F-639 | Ph | i-C₃H₇ | CH₃ | CH₂Ph |
| F-640 | COCH₃ | i-C₃H₇ | CH₃ | CH₂Ph |
| F-641 | COPh | i-C₃H₇ | CH₃ | CH₂Ph |
| F-642 | H | CH₂Ph | CH₃ | CH₂Ph |
| F-643 | CH₃ | CH₂Ph | CH₃ | CH₂Ph |
| F-644 | CH₂CH₃ | CH₂Ph | CH₃ | CH₂Ph |
| F-645 | CH₂CH═CH₂ | CH₂Ph | CH₃ | CH₂Ph |
| F-646 | CH₂Ph | CH₂Ph | CH₃ | CH₂Ph |
| F-647 | Ph | CH₂Ph | CH₃ | CH₂Ph |
| F-648 | COCH₃ | CH₂Ph | CH₃ | CH₂Ph |
| F-649 | COPh | CH₂Ph | CH₃ | CH₂Ph |
| F-650 | H | CH₂CH₂ | | CH₂Ph |
| F-651 | CH₃ | CH₂CH₂ | | CH₂Ph |
| F-652 | CH₂CH═CH₂ | CH₂CH₂ | | CH₂Ph |
| F-653 | CH₂Ph | CH₂CH₂ | | CH₂Ph |
| F-654 | Ph | CH₂CH₂ | | CH₂Ph |
| F-655 | COCH₃ | CH₂CH₂ | | CH₂Ph |
| F-656 | COPh | CH₂CH₂ | | CH₂Ph |
| F-657 | H | CH₂H₂CH₂ | | CH₂Ph |
| F-658 | CH₃ | CH₂H₂CH₂ | | CH₂Ph |
| F-659 | CH₂CH═CH₂ | CH₂H₂CH₂ | | CH₂Ph |
| F-660 | CH₂Ph | CH₂H₂CH₂ | | CH₂Ph |
| F-661 | Ph | CH₂H₂CH₂ | | CH₂Ph |
| F-662 | COCH₃ | CH₂H₂CH₂ | | CH₂Ph |
| F-663 | COPh | CH₂H₂CH₂ | | CH₂Ph |
| F-664 | H | CH₂CH₂CH₂CH₂ | | CH₂Ph |
| F-665 | CH₃ | CH₂CH₂CH₂CH₂ | | CH₂Ph |
| F-666 | CH₂CH═CH₂ | CH₂CH₂CH₂CH₂ | | CH₂Ph |
| F-667 | CH₂Ph | CH₂CH₂CH₂CH₂ | | CH₂Ph |
| F-668 | Ph | CH₂CH₂CH₂CH₂ | | CH₂Ph |
| F-669 | COCH₃ | CH₂CH₂CH₂CH₂ | | CH₂Ph |
| F-670 | COPh | CH₂CH₂CH₂CH₂ | | CH₂Ph |
| F-671 | H | CH₂CH₂CH₂CH₂CH₂ | | CH₂Ph |
| F-672 | CH₃ | CH₂CH₂CH₂CH₂CH₂ | | CH₂Ph |
| F-673 | CH₂CH═CH₂ | CH₂CH₂CH₂CH₂CH₂ | | CH₂Ph |
| F-674 | CH₂Ph | CH₂CH₂CH₂CH₂CH₂ | | CH₂Ph |
| F-675 | Ph | CH₂CH₂CH₂CH₂CH₂ | | CH₂Ph |
| F-676 | COCH₃ | CH₂CH₂CH₂CH₂CH₂ | | CH₂Ph |
| F-677 | COPh | CH₂CH₂CH₂CH₂CH₂ | | CH₂Ph |
| F-678 | H | H | H | C₂H₄O(C═O)CH₃ |
| F-679 | CH₃ | H | H | C₂H₄O(C═O)CH₃ |
| F-680 | CH₂CH₃ | H | H | C₂H₄O(C═O)CH₃ |
| F-681 | CH₂CH═CH₂ | H | H | C₂H₄O(C═O)CH₃ |
| F-682 | CH₂Ph | H | H | C₂H₄O(C═O)CH₃ |
| F-683 | Ph | H | H | C₂H₄O(C═O)CH₃ |
| F-684 | COCH₃ | H | H | C₂H₄O(C═O)CH₃ |
| F-685 | COPh | H | H | C₂H₄O(C═O)CH₃ |
| F-686 | H | CH₃ | H | C₂H₄O(C═O)CH₃ |
| F-687 | CH₃ | CH₃ | H | C₂H₄O(C═O)CH₃ |
| F-688 | CH₂CH₃ | CH₃ | H | C₂H₄O(C═O)CH₃ |
| F-689 | CH₂CH═CH₂ | CH₃ | H | C₂H₄O(C═O)CH₃ |
| F-690 | CH₂Ph | CH₃ | H | C₂H₄O(C═O)CH₃ |
| F-691 | Ph | CH₃ | H | C₂H₄O(C═O)CH₃ |
| F-692 | COCH₃ | CH₃ | H | C₂H₄O(C═O)CH₃ |
| F-693 | COPh | CH₃ | H | C₂H₄O(C═O)CH₃ |
| F-694 | H | CH₃ | CH₃ | C₂H₄O(C═O)CH₃ |
| F-695 | CH₃ | CH₃ | CH₃ | C₂H₄O(C═O)CH₃ |
| F-696 | CH₂CH₃ | CH₃ | CH₃ | C₂H₄O(C═O)CH₃ |
| F-697 | CH₂CH═CH₂ | CH₃ | CH₃ | C₂H₄O(C═O)CH₃ |
| F-698 | CH₂Ph | CH₃ | CH₃ | C₂H₄O(C═O)CH₃ |
| F-699 | Ph | CH₃ | CH₃ | C₂H₄O(C═O)CH₃ |
| F-700 | COCH₃ | CH₃ | CH₃ | C₂H₄O(C═O)CH₃ |
| F-701 | COPh | CH₃ | CH₃ | C₂H₄O(C═O)CH₃ |

| | | | |
|---|---|---|---|
| F-702 | H | CH₂CH₂ | C₂H₄O(C═O)CH₃ |
| F-703 | CH₃ | CH₂CH₂ | C₂H₄O(C═O)CH₃ |
| F-704 | H | CH₂H₂CH₂ | C₂H₄O(C═O)CH₃ |
| F-705 | CH₃ | CH₂H₂CH₂ | C₂H₄O(C═O)CH₃ |
| F-706 | H | CH₂CH₂CH₂CH₂ | C₂H₄O(C═O)CH₃ |
| F-707 | CH₃ | CH₂CH₂CH₂CH₂ | C₂H₄O(C═O)CH₃ |
| F-708 | H | CH₂CH₂CH₂CH₂CH₂ | C₂H₄O(C═O)CH₃ |
| F-709 | CH₃ | CH₂CH₂CH₂CH₂CH₂ | C₂H₄O(C═O)CH₃ |

TABLE 7
1H-NMR spectral details for representative Examples
from the above Tables. Spectra were measured in
deuterochloroform unless otherwise stated.

| Cpd | 1H-NMR |
|---|---|
| A-86 | 1.57(s, 6H), 7.66(d, 1H), 8.15(brs, 1H), 8.92(d, 1H), |
| | 9.37(s, 1H), 11.10(brs, 1H) |
| A-101 | 0.93(d, 3H), 1.07(d, 3H), 1.57(s, 3H), |
| | 2.1-.2.3(m, 1H), 7.65(d, 1H), 8.91(d, 1H), 9.32(s, 1H) |
| A-121 | 1.12(s, 9H), 1.59(s, 3H), 7.46(brs, 1H), 7.66(d, 1H), |
| | 8.92(d, 1H), 9.36(s, 1H), 11.22(brs, 1H) |
| A-146 | 0.8-1.1(m, 7H), 1.9-2.1(m, 2H), 2.1-2.3(m, 1H), |
| | 7.65(d, 1H), 8.91(d, 1H), 9.31(s, 1H), 11.17(brs, 1H) |
| A-196 | 1.4-2.1(m, 10H), 7.66(d, 1H), 8.36(brs, 1H), |
| | 8.92(d, 1H), 9.37(s, 1H) |
| A-91 | 0.94(t, 3H), 1.59(s, 3H), 1.8-2.1(m, 2H), |
| | 7.66(s, 1H), 8.47(brs, 1H), 8.96(d, 1H), 9.36(s, 1H) |
| B-121 | 1.11(s, 9H), 1.57(s, 3H), 6.12(brs, 1H), 7.64(d, 1H), |
| | 8.90(d, 1H), 9.33(s, 1H), 10.67(brs, 1H) |
| B-201 | 3.18(s, 3H), 4.22(s, 2H), 7.61(s, 1H), 8.84(d, 1H), |
| | 9.2-9.4(m, 2H) |
| B-206 | 1.57(d, 3H), 3.18(s, 3H), 4.30(q, 1H), 7.62(d, 1H), |
| | 8.85(1H, d), 9.26(s, 1H), 9.34(brs, 1H) |
| B-221 | 0.99(d, 3H), 1.13(d, 3H), 2.2-2.5(m, 1H), 3.16(s, 3H), |
| | 4.12(d, 1H), 7.62(d, 1H), 8.85(d, 1H), 9.2-9.4(m, 2H) |
| B-246 | 2.9-3.1(m, 4H), 3.2-3.4(m, 1H), 4.4-4.5(m, 1H), |
| | 7.2-7.4(m, 5H), 7.58(d, 1H), 8.82(d, 1H), 9.15(s, 1H), |
| | 9.25(brs, 1H) |
| B-286 | 1.54(s, 6H), 3.18(s, 3H), 7.62(d, 1H), 8.85(d, 1H), |
| | 9.29(s, 1H), 9.33(brs, 1H) |
| B-439 | 1.46(s, 6H), 1.73(s, 9H), 7.62(d, 1H), 8.83(d, 1H), |
| | 9.24(s, 1H), 9.94(brs, 1H) |
| B-455 | 4.22(s, 2H), 4.3-4.4(m, 2H), 5.8-6.0(m, 1H), |
| | 7.61(d, 1H), 8.84(d, 1H), 9.22(s, 1H),9.28(brs, 1H) |
| B-456 | 1.57(d, 3H), 4.2-4.4(m, 3H), 5.2-5.4(m, 2H), |
| | 5.8-6.0(m, 1H), 7.61(d, 1H), 8.84(d, 1H), 9.24(s, 1H), |
| | 9.35(brs, 1H) |
| B-459 | 0.99(d, 3H), 1.12(d, 3H), 2.2-2.5(m, 1H), 4.1-4.2(m, 1H), |
| | 4.2-4.4(m, 2H), 5.2-5.5(m, 2H), 5.7-6.0(m, 1H), |
| | 7.61(d, 1H), 8.84(d, 1H), 9.2-9.4(m, 2H) |
| B-464 | 3.0-3.4(m, 2H), 4.1-4.2(m, 2H), 4.4-4.6(m, 1H), |
| | 4.9-5.2(m, 2H), 5.5-5.7(m, 1H), 7.1-7.4(m, 5H), |
| | 7.58(d, 1H), 8.82(d, 1H), 9.14(s, 1H), 9.31(brs, 1H) |
| B-472 | 1.54(s, 6H), 4.2-4.3(m, 2H), 5.2-5.3(m, 2H), |
| | 5.8-6.0(m, 1H), 7.61(d, 1H), 8.83(d, 1H), 9.27(s, 1H), |
| | 9.35(brs, 1H) |
| B-527 | 1.52(s, 6H), 4.80(s, 3H), 7.2-7.5(m, 4H), 7.66(d, 1H), |
| | 8.88(d, 1H), 9.23(s, 1H), 9.40(brs, 1H) |
| B-536 | 3.79(s, 3H), 4.31(s, 2H), 4.44(s, 2H), 7.59(d, 1H), |
| | 8.84(d, 1H), 9.15(s, 1H), 9.28(brs, 1H) |
| B-541 | 1.45(s, 6H), 1.87(s, 6H), 4.70(s, 2H), 5.03(s, 2H), |
| | 7.1-7.4(m, 5H), 7.59(d, 1H), 8.83(d, 1H), 9.18(s, 1H), |
| | 9.56(brs, 1H) |
| B-86 | 1.58(s, 6H), 7.64(d, 1H), 8.89(d, 1H), 9.32(s, 1H), |
| | 10.58(brs, 1H) |
| B-87 | 1.61(s, 6H), 3.14(s, 3H), 7.65(d, 1H), 8.88(d, 1H), |
| | 9.26(s, 1H) |
| B-91 | 0.93(t, 3H), 1.56(s, 3H), 1.7-2.1(m, 2H), 6.37(brs, 1H), |
| | 7.64(d, 1H), 8.89(d, 1H), 9.30(s, 1H), 10.65(brs, 1H) |
| E-129 | 1.48(s, 6H), 2.62(s, 3H), 7.63(d, 1H), 8.88(d, 1H), |
| | 9.36(s, 1H), 11.23(brs, 1H) |
| E-130 | 1.54(s, 6H), 2.58(s, 3H), 3.19(s, 3H), 7.62(d, 1H), |
| | 8.84(d, 1H), 9.23(s, 1H) |
| E-135 | 1.74(s, 6H), 2.44(s, 6H), 7.61(d, 1H), 8.84(d, 1H), |
| | 9.04(s, 1H) |
| E-137 | 1.47(s, 6H), 3.84(d, 2H), 5.2-5.4(m, 2H), |
| | 5.8-6.1(m, 1H), 7.63(d, 1H), 8.87(d, 1H), 9.36(s, 1H), |
| | 11.18(brs, 1H) |
| E-137 | 0.73(t, 3H), 1.58(s, 3H), 1.8-2.0(m, 2H), 2.63(s, 3H), |
| | 7.62(d, 1H), 8.87(d, 1H), 9.34(s, 1H), 11. 22(brs, 1H) |
| E-138 | 0.68(t, 3H), 1.50(s, 3H), 1.8-2.2(m, 2H), 2.60(s, 1H), |
| | 3.18(s, 3H), 7.62(d, 1H), 8.84(d, 1H), 9.23(s, 1H) |
| E-143 | 0.71(t, 3H), 1.72(s, 3H), 2.1-2.3(m, 2H), 2.41(s, 3H), |
| | 2.45(s, 3H), 7.61(d, 1H), 8.83(d, 1H), 9.05(s, 1H) |
| E-185 | 1.06(s, 9H), 1.43(s, 3H), 2.62(s, 3H), 7.63(d, 1H), |
| | 8.87(d, 1H), 9.40(s, 1H), 11.39(brs, 1H) |
| E-325 | 1.3-2.0(m, 10H), 2.73(s, 3H), 7.58(d, 1H), |
| | 8.81(d, 1H), 9.29(s, 1H), 10.54(brs, 1H) |
| E-694 | 1.47(s, 6H), 2.17(s, 3H), 3.45(t, 2H), 4.38(t, 2H), |
| | 7.63(d, 1H), 8.88(d, 1H), 9.36(s, 1H), 11.18(brs,1H) |

According to a further feature of the present invention there is provided a method for the control of pests at a locus which comprises the application of an effective amount of a compound of formula (I) or a salt thereof. For this purpose, the said compound is normally used in the form of a pesticidal composition (i.e. in association with compatible diluents or carriers and/or surface active agents suitable for use in pesticidal compositions), for example as hereinafter described.

The term “compound of the invention” as used hereinafter embraces a 3-pyridylcarboxamide of formula (I) as defined above and a pesticidally acceptable salt thereof.

One aspect of the present invention as defined above is a method for the control of pests at a locus. The locus includes, for example, the pest itself, the place (plant, field, forest, orchard, waterway, soil, plant product, or the like) where the pest resides or feeds, or a place susceptible to future infestation by the pest. The compound of the invention may therefore be applied directly to the pest, to the place where the pest resides or feeds, or to the place susceptible to future infestation by the pest. As is evident from the foregoing pesticidal uses, the present invention provides pesticidally active compounds and methods of use of said compounds for the control of a number of pest species which includes: arthropods, especially insects or mites, or plant nematodes. The compound of the invention may thus be advantageously employed in practical uses, for example, in agricultural or horticultural crops, in forestry, in veterinary medicine or livestock husbandry, or in public health.

The compounds of the invention may be used for example in the following applications and on the following pests:

For the control of soil insects, such as corn rootworm, termites (especially for protection of structures), root maggots, wireworms, root weevils, stalkborers, cutworms, root aphids, or grubs. They may also be used to provide activity against plant pathogenic nematodes, such as root-knot, cyst, dagger, lesion, or stem or bulb nematodes, or against mites. For the control of soil pests, for example corn rootworm, the compounds are advantageously applied to or incorporated at an effective rate into the soil in which crops are planted or to be planted or to the seeds or growing plant roots.

In the area of public health, the compounds are especially useful in the control of many insects, especially filth flies or other Dipteran pests, such as houseflies, stableflies, soldierflies, hornflies, deerflies, horseflies, midges, punkies, blackflies, or mosquitoes.

In the protection of stored products, for example cereals, including grain or flour, groundnuts, animal feedstuffs, timber or household goods, e.g. carpets and textiles, compounds of the invention are useful against attack by arthropods, more especially beetles, including weevils, moths or mites, for example Ephestia spp. (flour moths), Anthrenus spp. (carpet beetles), Tribolium spp. (flour beetles), Sitophilus spp. (grain weevils) or Acarus spp. (mites).

In the control of cockroaches, ants or termites or similar arthropod pests in infested domestic or industrial premises or in the control of mosquito larvae in waterways, wells, reservoirs or other running or standing water.

For the treatment of foundations, structures or soil in the prevention of the attack on building by termites, for example, Reticulitermes spp., Heterotermes spp., Coptotermes spp.

In agriculture against adults, larvae and eggs of Lepidoptera (butterflies and moths), e.g. Heliothis spp. such as Heliothis virescens (tobacco budworm), Heliothis armigera and Heliothis zea. Against adults and larvae of Coleoptera (beetles) e.g. Anthonomus spp. e.g. grandis (cotton boll weevil), Leptinotarsa decemlineata (Colorado potato beetle), Diabrotica spp. (corn rootworms). Against Heteroptera (Hemiptera and Homoptera) e.g. Psylla spp., Bemisia spp., Trialeurodes spp., Aphis spp., Myzus spp., Megoura viciae, Phylloxera spp., Nephoteftix spp. (rice leaf hoppers), Nilaparvata spp., Pseudococcus spp., and Coccus spp. Against Diptera e.g. Musca spp. Against Thysanoptera such as Thrips tabaci. Against Orthoptera such as Locusta and Schistocerca spp., (locusts and crickets) e.g. Gryllus spp., and Acheta spp. for example, Blatta orientalis, Periplaneta americana, Blatella germanica, Locusta migratoria migratorioides, and Schistocerca gregaria. Against Collembola e.g. Periplaneta spp. and Blatella spp. (roaches). Against arthropods of agricultural significance such as Acari (mites) e.g. Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Eotetranychus spp., Oligonychus spp., Eutetranychus spp.

From the order of the Isopoda, for example, Oniscus aselus, Armadium vulgare, Porcellio scaber.

Against nematodes which attack plants or trees of importance to agriculture, forestry or horticulture either directly or by spreading bacterial, viral, mycoplasma or fungal diseases of the plants. The plant-parasitic nematodes which can be controlled in accordance with the invention include, for example, the root-parasitic soil-dwelling nematodes such as, for example, those of the genera Meloidogyne (root knot nematodes, such as Meloidogyne incognita, Meloidogyne hapla and Meloidogyne javanica), Heterodera and Globodera (cyst-forming nematodes, such as Globodera rostochiensis, Globodera pallida, Heterodera trifolii) and of the genera Radopholus, such as Radopholus similis, Pratylenchus such as Pratylenchus neglectus, Pratylenchus penetrans and Pratylenchus curvitatus;

Tylenchulus such as Tylenchulus semipenetrans, Tylenchorhynchus, such as Tylenchorhynchus dubius and Tylenchorhynchus claytoni, Rotylenchus such as Rotylenchus robustus, Heliocotylenchus such as Haliocotylenchus multicinctus, Belonoaimnus such as Belonoaimus longicaudatus, Longidorus such as Longidorus elongatus, Trichodorus such as Trichodorus primitivus and Xiphinema such as Xiphinema index.

Other nematode genera which can be controlled using the compounds according to the invention are Ditylenchus (stem parasites, such as Ditylenchus dipsaci and Ditylenchus destructor), Aphelenchoides (foliar nematodes, such as Aphelenchoides ritzemabosi) and Anguina (seed nematodes, such as Anguina tritici).

In the field of veterinary medicine or livestock husbandry or in the maintenance of public health against arthropods which are parasitic internally or externally upon vertebrates, particularly warm-blooded vertebrates, for example domestic animals, e.g. cattle, sheep, goats, equines, swine, poultry, dogs or cats, for example Acarina, including ticks (e.g. Ixodes spp., Boophilus spp. e.g. Boophilus microplus, Rhipicephalus spp. e.g. Rhipicephalus appendiculatus Ornithodorus spp. (e.g. Ornithodorus moubata) and mites (e.g. Damalinia spp.); fleas (e.g. Ctenocephalides felis and Ctenocephalides canis); Diptera (e.g. Aedes spp., Anopheles spp., Musca spp., Hypoderma spp.); Hemiptera.; Dictyoptera (e.g. Periplaneta spp., Blatella spp.); Hymenoptera; for example against infections of the gastro-intestinal tract caused by parasitic nematode worms, for example members of the family Trichostrongylidae.

From the class of the helminths, for example, Haemonchus, Trichostrongulus, Ostertagia, Cooperia, Chabertia, Strongyloides, Oesophagostomum, Hyostrongulus, Ancylostoma, Ascaris and Heterakis and also Fasciola.

From the class of the Gastropoda, for example, Deroceras spp., Arion spp., Lymnaea spp., Galba spp., Succinea spp., Biomphalaria spp., Bulinus spp., Oncomelania spp.

From the class of the Bivalva, for example, Dreissena spp.

Furthermore against Protozoa such as Eimeria.

The preferred insect species which are controlled are sucking insect pests such as aphids (e.g. Aphis fabae, Aphis pomi, Aphis spiraecola, Aphis gossypii, Aphis nasturtii, Dysaphis plantaginea, Eriosoma spp., Rhopalosiphum padi, Acyrthosiphon pisum, Pemphigus bursarius, Myzus persicae, Myzus nicotianae, Myzus euphorbiae, Phylloxera spp., Toxoptera spp, Brevicoryne brassicae, Macrosiphum avenae, Macrosiphum euphorbiae, Nasonovia ribisnigri, Sitobion avenae, Brachycaudus helychrysii or Phorodon humuli), cicadas (Idioscopus clypealis, Scaphoides titanus, Empoasca onuki, Empoasca vitis, Empoasca devastans, Empoasca libyca, Empoasca biguttula, Empoasca facialis or Erythroneura spp), thrips (Hercinothrips femoralis, Scirtothrips aurantii, Scirtothrips dorsalis, Frankliniella schultzei, Frankliniella fusca, Frankliniella occidentalis, Frankliniella tritici, Kakothrips spp., Thrips oryzae, Thrips palmi or Thrips tabaci) or whiteflies (Aleurodes brassicae, Bemisia tabaci, Trialeurodes vaporariorum or Aleurodes proletella), or mealybugs (Dysmicoccus spp., Planococcus spp. or Phenacoccus spp.).

In practical use for the control of arthropods, especially insects or acarids, or nematode pests of plants, a method, for example, comprises applying to the plants or to the medium in which they grow an effective amount of a compound of the invention. For such a method, the compound of the invention is generally applied to the locus in which the arthropod or nematode infestation is to be controlled at an effective rate in the range of about 2 g to about 1 kg of the active compound per hectare of locus treated. Under ideal conditions, depending on the pest to be controlled, a lower rate may offer adequate protection. On the other hand, adverse weather conditions, resistance of the pest or other factors may require that the active ingredient be used at higher rates. The optimum rate depends usually upon a number of factors, for example, the type of pest being controlled, the type or the growth stage of the infested plant, the row spacing or also the method of application. Preferably an effective rate range of the active compound is from about 10 g/ha to about 400 g/ha, more preferably from about 50 g/ha to about 200 g/ha.

When a pest is soil-borne, the active compound generally in a formulated composition, is distributed evenly over the area to be treated (ie, for example broadcast or band treatment) in any convenient manner and is applied at rates from about 10 g/ha to about 400 g ai/ha, preferably from about 50 g/ha to about 200 g ai/ha. When applied as a root dip to seedlings or drip irrigation to plants the liquid solution or suspension contains from about 0.075 to about 1000 mg ai/l, preferably from about to about 200 mg ai/l. Application may be made, if desired, to the field or crop-growing area generally or in close proximity to the seed or plant to be protected from attack. The compound of the invention can be washed into the soil by spraying with water over the area or can be left to the natural action of rainfall. During or after application, the formulated compound can, if desired, be distributed mechanically in the soil, for example by ploughing, disking, or use of drag chains. Application can be prior to planting, at planting, after planting but before sprouting has taken place, or after sprouting.

The compound of the invention and methods of control of pests therewith are of particular value in the protection of field, forage, plantation, glasshouse, orchard or vineyard crops, of ornamentals, or of plantation or forest trees, for example: cereals (such as wheat, barley, rye, oats, millet or rice), cotton, vegetables (such as peppers, potatoes, tomatoes or peas), field crops (such as sugar beets, soybeans or oil seed rape), grassland or forage crops (such as maize or sorghum), cassaya, orchards or groves (such as of stone or pit fruit or citrus), ornamental plants, flowers or vegetables or shrubs under glass or in gardens or parks, or forest trees (both deciduous and evergreen) in forests, plantations or nurseries.

They are also valuable in the protection of timber (standing, felled, converted, stored or structural) from attack, for example, by sawflies or beetles or termites.

They have applications in the protection of stored products such as grains, fruits, nuts, spices or tobacco, whether whole, milled or compounded into products, from moth, beetle, mite or grain weevil attack. Also protected are stored animal products such as skins, hair, wool or feathers in natural or converted form (e.g. as carpets or textiles) from moth or beetle attack as well as stored meat, fish or grains from beetle, mite or fly attack.

Additionally, the compound of the invention and methods of use thereof are of particular value in the control of arthropods or helminths which are injurious to, or spread or act as vectors of diseases domestic animals, for example those hereinbefore mentioned, and more especially in the control of ticks, mites, lice, fleas, midges, or biting, nuisance or myiasis flies. The compounds of the invention are particularly useful in controlling arthropods or helminths which are present inside domestic host animals or which feed in or on the skin or suck the blood of the animal, for which purpose they may be administered orally, parenterally, percutaneously or topically.

The compositions hereinafter described for application to growing crops or crop growing loci or as a seed dressing may, in general, alternatively be employed in the protection of stored products, household goods, property or areas of the general environment. Suitable means of applying the compounds of the invention include:
to growing crops as foliar sprays (for example as an in-furrow spray), dusts, granules, fogs or foams or also as suspensions of finely divided or encapsulated compositions as soil or root treatments by liquid drenches, dusts, granules, smokes or foams; to seeds of crops via application as seed dressings by liquid slurries or dusts;
to animals infested by or exposed to infestation by arthropods or helminths, by parenteral, oral or topical application of compositions in which the active ingredient exhibits an immediate and/or prolonged action over a period of time against the arthropods or helminths, for example by incorporation in feed or suitable orally-ingestible pharmaceutical formulations, edible baits, salt licks, dietary supplements, pour-on formulations, sprays, baths, dips, showers, jets, dusts, greases, shampoos, creams, wax smears or livestock self-treatment systems;
to the environment in general or to specific locations where pests may lurk, including stored products, timber, household goods, or domestic or industrial premises, as sprays, fogs, dusts, smokes, wax-smears, lacquers, granules or baits, or in tricklefeeds to waterways, wells, reservoirs or other running or standing water.

The compounds of the formula (I) can also be employed for controlling harmful organisms in crops of known genetically engineered plants or genetically engineered plants yet to be developed. As a rule, the transgenic plants are distinguished by especially advantageous properties, for example by resistances to particular crop protection agents, resistances to plant diseases or pathogens of plant diseases, such as particular insects or microorganisms such as fungi, bacteria or viruses. Other particular properties concern, for example, the harvested material with regard to quantity, quality, storage properties, composition and specific constituents. Thus, transgenic plants are known where the starch content is increased, or the starch quality is altered, or where the harvested material has a different fatty acid composition.

The use in economically important transgenic crops of useful plants and ornamentals is preferred, for example of cereals such as wheat, barley, rye, oats, millet, rice, cassaya and maize or else crops of sugar beet, cotton, soya, oilseed rape, potatoes, tomatoes, peas and other types of vegetables.

When used in transgenic crops, in particular those which have resistances to insects, effects are frequently observed, in addition to the effects against harmful organisms to be observed in other crops, which are specific for application in the transgenic crop in question, for example an altered or specifically widened spectrum of pests which can be controlled, or altered application rates which may be employed for application.

The invention therefore also relates to the use of compounds of the formula (I) for controlling harmful organisms in transgenic crop plants.

In addition to their lethal effect on pests, the compounds of the formula (I) also have a pronounced repellent effect.

A repellent for the purpose of the invention is a substance or substance mixture which has a warding-off or fending-off effect on other life organisms, in particular harmful pests and nuisance pests. The term also encompasses effects such as the antifeeding effect, where the intake of feed is disturbed or prevented (antifeedant effect), supression of oviposition, or an effect on the development of the population.

The invention therefore also provides the use of compounds of the formula (I) or their salts for achieving the abovementioned effects, in particular in the case of the pests stated in the biological examples.

The invention also provides a method for fending off, or warding off, harmful organisms, where one or more compounds of the formula (I) or their salts are applied to the site from which the harmful organisms are to be fended off or warded off.

In the case of a plant, application may mean, for example, a treatment of the plant, or also of the seed.

As regards the effect on populations, it is interesting to note that effects can also be observed in succession during the development of a population, where summation may take place. In such a case, the individual effect itself may only have an efficacy of markedly less than 100% but in total an efficacy of 100% is still achieved in the end.

Moreover, the compounds of the formula (I) or their salts are distinguished by the fact that the composition is usually applied earlier than in the case of a direct control, if the abovementioned effects are to be exploited. The effect frequently lasts over a long period, so that a duration of action of over 2 months is achieved.

The effects are observed in insects, arachnids and others of the abovementioned pests.

According to a further feature of the present invention there is provided a pesticidal composition comprising one or more compounds of the invention as defined above, in association with, and preferably homogeneously dispersed in one or more compatible pesticidally acceptable diluents or carriers and/or surface active agents [i.e. diluents or carriers and/or surface active agents of the type generally accepted in the art as being suitable for use in pesticidal compositions and which are compatible with compounds of the invention].

In practice, the compounds of the invention most-frequently form parts of compositions. These compositions can be employed to control arthropods, especially insects and acarids, or helminths such as plant nematodes. The compositions may be of any type known in the art suitable for application to the desired pest in any premises or indoor or outdoor area. These compositions contain at least one compound of the invention as the active ingredient in combination or association with one or more other compatible components which are for example, solid or liquid carriers or diluents, adjuvants, surface-active-agents, or the like appropriate for the intended use and which are agronomically or medicinally acceptable. These compositions, which may be prepared by any manner known in the art, likewise form a part of this invention.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

The formulations generally comprise between 0.1 and 95% by weight of active compound, preferably between 0.5 and 90%.

The active compound according to the invention can be present in its commercially available formulations and in the use forms prepared from these formulations as a mixture with other active compounds, such as insecticides, attractants, sterilants, bactericides, acaricides, nematicides, fungicides, safeners, growth-regulating compounds or herbicides. The insecticides include, for example, phosphoric esters, carbamates, carboxylic esters, chlorinated hydrocarbons, phenylureas, compounds produced by microorganisms, inter alia.

Particularly suitable mixing components are, for example, the following compounds:

Fungicides:

2-phenylphenol; 8-hydroxyquinoline sulfate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metlalaxyl-M; metconazole; metho-sulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrin; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)-N-[2-[4-[[3-(4-chlorophenyl)-2-propynyl]oxy]-3-methoxyphenyl]ethyl]-3-methyl-2-[(methylsulfonyl)amino]butanamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl(2,3-dihydro-2,2-dimethyl-1H-inden-1-yl)-1H-imidazole-5-carboxylate; monopotassium carbonate; N-(6-methoxy-3-pyridinyl)-cyclopropanecarboxamide; N-butyl-8-(1,1-dimethylethyl)-1-oxaspiro[4.5]decane-3-amine; sodium tetrathiocarbonate;
and copper salts and preparations, such as Bordeaux mixture; copper hydroxide; copper naphthenate; copper oxychloride; copper sulfate; cufraneb; copper oxide; mancopper; oxine-copper.
   Bactericides:
bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulphate and other copper preparations.

Insecticides/Acaricides/Nematicides:
abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alpha-cypermethrin (alphamethrin), amidoflumet, aminocarb, amitraz, avermectin, AZ-60541, azadirachtin, azamethiphos, azinphos-methyl, azinphos-ethyl, azocyclotin,
Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-S-cyclopentyl-isomer, bioethanomethrin, biopermethrin, bioresmethrin, bistrifluron, BPMC, brofenprox, bromophos-ethyl, bromopropylate, bromfenvinfos (-methyl), BTG-504, BTG-505, bufencarb, buprofezin, butathiofos, butocarboxim, butoxycarboxim, butylpyridaben,
cadusafos, camphechlor, carbaryl, carbofuran, carbophenothion, carbosulfan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos (-ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, Cydia pomonella, cyfluthrin, cyhalothrin, cyhexatin, cypermethrin, cyphenothrin (1R-trans-isomer), cyromazine,
DDT, deltamethrin, demeton-S-methyl, demeton-S-methylsulphone, diafenthiuron, dialifos, diazinon, dichlofenthion, dichlorvos, dicofol, dicrotophos, dicyclanil, diflubenzuron, dimethoate, dimethylvinphos, dinobuton, dinocap, dinetofuran, diofenolan, disulfoton, docusat-sodium, dofenapyn, DOWCO-439,
eflusilanate, emamectin, emamectin-benzoate, empenthrin (1R-isomer), endosulfan, Entomopthora spp., EPM, esfenvalerate, ethiofencarb, ethiprole, ethion, ethoprophos, etofenprox, etoxazole, etrimfos,
famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb,
gamma-HCH, gossyplure, grandlure, granulosis viruses,
halfenprox, halofenozide, HCH, HCN-801, heptenophos, hexaflumuron, hexythiazox, hydramethylnone, hydroprene,
IKA-2002, imidacloprid, imiprothrin, indoxacarb, iodofenphos, iprobenfos, isazofos, isofenphos, isoprocarb, isoxathion, ivermectin,
japonilure,
kadethrin, nuclear polyhedrosis viruses, kinoprene,
lambda-cyhalothrin, lindane, lufenuron,
malathion, mecarbam, mesulfenfos, metaldehyde, metam-sodium, methacrifos, methamidophos, Metharhizium anisopliae, Metharhizium flavoviride, methidathion, methiocarb, methomyl, methoprene, methoxychlor, methoxyfenozide, metolcarb, metoxadiazone, mevinphos, milbemectin, milbemycin, MKI-245, MON-45700, monocrotophos, moxidectin, MTI-800,
naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron,
OK-5101, OK-5201, OK-9601, OK-9602, OK-9701, OK-9802, omethoate, oxamyl, oxydemeton-methyl,
Paecilomyces fumosoroseus, parathion-methyl, parathion (-ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos-methyl, pirimiphos-ethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen,
quinalphos,
resmethrin, RH-5849, ribavirin, RU-12457, RU-15525,
S-421, S-1833, salithion, sebufos, SI-0009, silafluofen, spinosad, spirodiclofen, spiromesifen, sulfluramid, sulfotep, sulprofos, SZI-121,
tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R-isomer), tetrasul, theta-cypermethrin, thiacloprid, thiamethoxam, thiapronil, thiatriphos, thiocyclam hydrogenoxalate, thiodicarb, thiofanox, thiometon, thiosultap-sodium, thuringiensin, tolfenpyrad, tralocythrin, tralomethrin, transfluthrin, triarathene, triazamate, triazophos, triazuron, trichlophenidine, trichlorfon, triflumuron, trimethacarb,
vamidothion, vaniliprole, verbutin, Verticillium lecanii,
WL-108477, WL-40027,
YI-5201, YI-5301, YI-5302,
XMC, xylylcarb,
ZA-3274, zeta-cypermethrin, zolaprofos, ZXI-8901,
the compound 3-methylphenyl propylcarbamate (tsumacide Z),
the compound 3-(5-chloro-3-pyridinyl)-8-(2,2,2-trifluoroethyl)-8-azabicyclo[3.2.1]-octane-3-carbonitrile (CAS-Reg. No. 185982-80-3) and the corresponding 3-endo-isomer (CAS-Reg. No. 185984-60-5) (cf. WO-96/37494, WO-98/25923),
and preparations which comprise insecticidally active plant extracts, nematodes, fungi or viruses.

The abovementioned components for combinations are known active substances, many of which are described in Ch. R Worthing, S. B. Walker, The Pesticide Manual, 12^{th} Edition, British Crop Protection Council, Farnham 2000.

The effective use doses of the compounds employed in the invention can vary within wide limits, particularly depending on the nature of the pest to be eliminated or degree of infestation, for example, of crops with these pests. In general, the compositions according to the invention usually contain about 0.05 to about 95% (by weight) of one or more active ingredients according to the invention, about 1 to about 95% of one or more solid or liquid carriers and, optionally, about 0.1 to about 50% of one or more other compatible components, such as surface-active agents or the like. In the present account, the term “carrier” denotes an organic or inorganic ingredient, natural or synthetic, with which the active ingredient is combined to facilitate its application, for example, to the plant, to seeds or to the soil. This carrier is therefore generally inert and it must be acceptable (for example, agronomically acceptable, particularly to the treated plant).

The carrier may be a solid, for ensample, clays, natural or synthetic silicates, silica, resins, waxes, solid fertilizers (for example ammonium salts), ground natural minerals, such as kaolins, clays, talc, chalk, quartz, attapulgite, montmorillonite, bentonite or diatomaceous earth, or ground synthetic minerals, such as silica, alumina, or silicates especially aluminium or magnesium silicates. As solid carriers for granules the following are suitable: crushed or fractionated natural rocks such as calcite, marble, pumice, sepiolite and dolomite; synthetic granules of inorganic or organic meals; granules of organic material such as sawdust, coconut shells, corn cobs, corn husks or tobacco stalks; kieselguhr, tricalcium phosphate, powdered cork, or absorbent carbon black; water soluble polymers, resins, waxes; or solid fertilizers. Such solid compositions may, if desired, contain one or more compatible wetting, dispersing, emulsifying or colouring agents which, when solid, may also serve as a diluent.

The carrier may also be liquid, for example: water; alcohols, particularly butanol or glycol, as well as their ethers or esters, particularly methylglycol acetate; ketones, particularly acetone, cyclohexanone, methylethyl ketone, methylisobutylketone, or isophorone; petroleum fractions such as paraffinic or aromatic hydrocarbons, particularly xylenes or alkyl naphthalenes; mineral or vegetable oils; aliphatic chlorinated hydrocarbons, particularly trichloroethane or methylene chloride; aromatic chlorinated hydrocarbons, particularly chlorobenzenes; water-soluble or strongly polar solvents such as dimethylformamide, dimethyl sulphoxide, or N-methylpyrrolidone; liquefied gases; or the like or a mixture thereof.

The surface-active agent may be an emulsifying agent, dispersing agent or wetting agent of the ionic or non-ionic type or a mixture of such surface-active agents. Amongst these are e.g., salts of polyacrylic acids, salts of lignosulphonic acids, salts of phenolsulphonic or naphthalenesulphonic acids, polycondensates of ethylene oxide with fatty alcohols or fatty acids or fatty esters or fatty amines, substituted phenols (particularly alkylphenols or arylphenols), salts of sulphosuccinic acid esters, taurine derivatives (particularly alkyltaurates), phosphoric esters of alcohols or of polycondensates of ethylene oxide with phenols, esters of fatty acids with polyols, or sulphate, sulphonate or phosphate functional derivatives of the above compounds. The presence of at least one surface-active agent is generally essential when the active ingredient and/or the inert carrier are only slightly water soluble or are not water soluble and the carrier agent of the composition for application is water. Compositions of the invention may further contain other additives such as adhesives or colorants. Adhesives such as carboxymethylcellulose or natural or synthetic polymers in the form of powders, granules or lattices, such as arabic gum, polyvinyl alcohol or polyvinyl acetate, natural phospholipids, such as cephalins or lecithins, or synthetic phospholipids can be used in the formulations. It is possible to use colorants such as inorganic pigments, for example: iron oxides, titanium oxides or Prussian Blue; organic dyestuffs, such as alizarin dyestuffs, azo dyestuffs or metal phthalocyanine dyestuffs; or trace nutrients such as salts of iron, manganese, boron, copper, cobalt, molybdenum or zinc.

For their agricultural application, the compounds of the invention are therefore generally in the form of compositions, which are in various solid or liquid forms.

Solid forms of compositions which can be used are dusting powders (with a content of the compound of the invention, ranging up to 80%), wettable powders or granules (including water dispersible granules), particularly those obtained by extrusion, compacting, impregnation of a granular carrier, or granulation starting from a powder (the content of the compound of the invention, in these wettable powders or granules being between about 0.5 and about 80%). Solid homogenous or heterogenous compositions containing one or more compounds of the invention, for example granules, pellets, briquettes or capsules, may be used to treat standing or running water over a period of time. A similar effect may be achieved using trickle or intermittent feeds of water dispersible concentrates as described herein.

Liquid compositions, for example, include aqueous or non-aqueous solutions or suspensions (such as emulsifiable concentrates, emulsions, flowables, dispersions, or solutions) or aerosols. Liquid compositions also include, in particular, emulsifiable concentrates, dispersions, emulsions, flowables, aerosols, wettable powders (or powder for spraying), dry flowables or pastes as forms of compositions which are liquid or intended to form liquid compositions when applied, for example as aqueous sprays (including low and ultra-low volume) or as fogs or aerosols.

Liquid compositions, for example, in the form of emulsifiable or soluble concentrates most frequently comprise about 5 to about 80% by weight of the active ingredient, while the emulsions or solutions which are ready for application contain, in their case, about 0.01 to about 20% of the active ingredient. Besides the solvent, the emulsifiable or soluble concentrates may contain, when required, about 2 to about 50% of suitable additives, such as stabilizers, surface-active agents, penetrating agents, corrosion inhibitors, colorants or adhesives. Emulsions of any required concentration, which are particularly suitable for application, for example, to plants, may be obtained from these concentrates by dilution with water. These compositions are included within the scope of the compositions which may be employed in the present invention. The emulsions may be in the form of water-in-oil or oil-in-water type and they may have a thick consistency.

The liquid compositions of this invention may, in addition to normal agricultural use applications be used for example to treat substrates or sites infested or liable to infestation by arthropods (or other pests controlled by compounds of this invention) including premises, outdoor or indoor storage or processing areas, containers or equipment or standing or running water.

All these aqueous dispersions or emulsions or spraying mixtures can be applied, for example, to crops by any suitable means, chiefly by spraying, at rates which are generally of the order of about 100 to about 1,200 liters of spraying mixture per hectare, but may be higher or lower (eg. low or ultra-low volume) depending upon the need or application technique. The compound or compositions according to the invention are conveniently applied to vegetation and in particular to roots or leaves having pests to be eliminated. Another method of application of the compounds or compositions according to the invention is by chemigation, that is to say, the addition of a formulation containing the active ingredient to irrigation water. This irrigation may be sprinkler irrigation for foliar pesticides or it can be ground irrigation or underground irrigation for soil or for systemic pesticides.

The concentrated suspensions, which can be applied by spraying, are prepared so as to produce a stable fluid product which does not settle (fine grinding) and usually contain from about 10 to about 75% by weight of active ingredient, from about 0.5 to about 30% of surface-active agents, from about 0.1 to about 10% of thixotropic agents, from about 0 to about 30% of suitable additives, such as anti-foaming agents, corrosion inhibitors, stabilizers, penetrating agents, adhesives and, as the carrier, water or an organic liquid in which the active ingredient is poorly soluble or insoluble Some organic solids or inorganic salts may be dissolved in the carrier to help prevent settling or as antifreezes for water.

The wettable powers (or powder for spraying) are usually prepared so that they contain from about 10 to about 80% by weight of active ingredient, from about 20 to about 90% of a solid carrier, from about 0 to about 5% of a wetting agent, from about 3 to about 10% of a dispersing agent and, when necessary, from about 0 to about 80% of one or more stabilizers and/or other additives, such as penetrating agents, adhesives, anti-caking agents, colorants, or the like. To obtain these wettable powders, the active ingredient is thoroughly mixed in a suitable blender with additional substances which may be impregnated on the porous filler and is ground using a mill or other suitable grinder. This produces wettable powders, the wettability and the suspendability of which are advantageous. They may be suspended in water to give any desired concentration and this suspension can be employed very advantageously in particular for application to plant foliage.

The “water dispersible granules (WG)” (granules which are readily dispersible in water) have compositions which are substantially close to that of the wettable powders. They may be prepared by granulation of formulations described for the wettable powders, either by a wet route (contacting finely divided active ingredient with the inert filler and a little water, e.g. 1 to 20% by weight, or with an aqueous solution of a dispersing agent or binder, followed by drying and screening), or by a dry route (compacting followed by grinding and screening).

The rates and concentrations of the formulated compositions may vary according to the method of application or the nature of the compositions or use thereof. Generally speaking, the compositions for application to control arthropod or helminth pests usually contain from about 0.00001% to about 95%, more particularly from about 0.0005% to about 50% by weight of one or more compounds of the invention, or of total active ingredients (that is to say the compounds of the invention, together with other substances toxic to arthropods or helminths, synergists, trace elements or stabilizers). The actual compositions employed and their rate of application will be selected to achieve the desired effect(s) by the farmer, livestock producer, medical or veterinary practitioner, pest control operator or other person skilled in the art. Solid or liquid compositions for application topically to animals, timber, stored products or household goods usually contain from about 0.00005% to about 90%, more particularly from about 0.001% to about 10%, by weight of one or more compounds of the invention. For administration to animals orally or parenterally, including percutaneously solid or liquid compositions, these normally contain from about 0.1% to about 90% by weight of one or more compounds of the invention. Medicated feedstuffs normally contain from about 0.001% to about 3% by weight of one or more compounds of the invention. Concentrates or supplements for mixing with feedstuffs normally contain from about 5% to about 90%, preferably from about 5% to about 50%, by weight of one or more compounds of the invention. Mineral salt licks normally contain from about 0.1% to about 10% by weight of one or more compounds of formula (I) or pesticidally acceptable salts thereof.

Dusts or liquid compositions for application to livestock, goods, premises or outdoor areas may contain from about 0.0001% to about 15%, more especially from about 0.005% to about 2.0%, by weight, of one or more compounds of the invention. Suitable concentrations in treated waters are between about 0.0001 ppm and about 20 ppm, more particularly about 0.001 ppm to about 5.0 ppm. of one or more compounds of the invention, and may be used therapeutically in fish farming with appropriate exposure times. Edible baits may contain from about 0.01% to about 5%, preferably from about 0.01% to about 1.0%, by weight, of one or more compounds of the invention.

When administered to vertebrates parenterally, orally or by percutaneous or other means, the dosage of compounds of the invention, will depend upon the species, age, or health of the vertebrate and upon the nature and degree of its actual or potential infestation by arthropod or helminth pests. A single dose of about 0.1 to about 100 mg, preferably about 2.0 to about 20.0 mg, per kg body weight of the animal or doses of about 0.01 to about 20.0 mg, preferably about 0.1 to about 5.0 mg, per kg body weight of the animal per day, for sustained medication, are generally suitable by oral or parenteral administration. By use of sustained release formulations or devices, the daily doses required over a period of months may be combined and administered to animals on a single occasion.

The following composition EXAMPLES 2A-2M illustrate compositions for use against arthropods, especially insects or acarids, or helminths such as plant nematodes, which comprise, as active ingredient, compounds of the invention, such as those described in preparative examples. The compositions described in EXAMPLES 2A-2M can each be diluted to give a sprayable compositon at concentrations suitable for use in the field. Generic chemical descriptions of the ingredients (for which all of the following percentages are in weight percent), used in the composition EXAMPLES 2A-2M exemplified below, are as follows:

| Trade Name | Chemical Description |
|---|---|
| Ethylan BCP | Nonylphenol ethylene oxide condensate |
| Soprophor BSU | Tristyrylphenol ethylene oxide condensate |
| Arylan CA | A 70% w/v solution of calcium |
| | dodecylbenzenesulfonate |
| Solvesso 150 | Light C₁₀ aromatic solvent |
| Arylan S | Sodium dodecylbenzenesulfonate |
| Darvan NO₂ | Sodium lignosulphonate |
| Celite PF | Synthetic magnesium silicate carrier |
| Sopropon T36 | Sodium salts of polycarboxylic acids |
| Rhodigel 23 | Polysaccharide xanthan gum |
| Bentone 38 | Organic derivative of magnesium montmorillonite |
| Aerosil | Microfine silicon dioxide |

### Example 2A

A water soluble concentrate is prepared with the composition as follows:

| | |
|---|---|
| Active ingredient | 7% |
| Ethylan BCP | 10% |
| N-methylpyrrolidone | 83% |

To a solution of Ethylan BCP dissolved in a portion of N-methylpyrrolidone is added the active ingredient with heating and stirring until dissolved. The resulting solution is made up to volume with the remainder of the solvent.

### Example 2B

An emulsifiable concentrate (EC) is prepared with the composition as follows:

| Active ingredient | 25%(max) |
|---|---|
| Soprophor BSU | 10% |
| Arylan CA | 5% |
| N-methylpyrrolidone | 50% |
| Solvesso 150 | 10% |

The first three components are dissolved in N-methylpyrrolidone and to this is then added the Solvesso 150 to give the final volume.

### Example 2C

A wettable powder (WP) is prepared with the composition as follows:

| | |
|---|---|
| Active ingredient | 40% |
| Arylan S | 2% |
| Darvan NO₂ | 5% |
| Celite PF | 53% |

The ingredients are mixed and ground in a hammer-mill to a powder with a particle size of less than 50 microns.

### Example 2D

An aqueous-flowable formulation is prepared with the composition as follows:

| | |
|---|---|
| Active ingredient | 40.00% |
| Ethylan BCP | 1.00% |
| Sopropon T360. | 0.20% |
| Ethylene glycol | 5.00% |
| Rhodigel 230. | 0.15% |
| Water | 53.65% |

The ingredients are intimately mixed and are ground in a bead mill until a mean particle size of less than 3 microns is obtained.

### Example 2E

An emulsifiable suspension concentrate is prepared with the composition as follows:

| | |
|---|---|
| Active ingredient | 30.0% |
| Ethylan BCP | 10.0% |
| Bentone 38 | 0.5% |
| Solvesso 150 | 59.5% |

The ingredients are intimately mixed and ground in a beadmill until a mean particle size of less than 3 microns is obtained.

### Example 2F

A water dispersible granule is prepared with the composition as follows:

| | |
|---|---|
| Active ingredient | 30% |
| Darvan No 2 | 15% |
| Arylan S | 8% |
| Celite PF | 47% |

The ingredients are mixed, micronized in a fluid-energy mill and then granulated in a rotating pelletizer by spraying with water (up to 10%). The resulting granules are dried in a fluid-bed drier to remove excess water.

### Example 2G

A dusting powder is prepared with the composition as follows:

| | |
|---|---|
| Active ingredient | 1 to 10% |
| Talc powder-superfine | 99 to 90% |

The ingredients are intimately mixed and further ground as necessary to achieve a fine powder. This powder may be applied to a locus of arthropod infestation, for example refuse dumps, stored products or household goods or animals infested by, or at risk of infestation by, arthropods to control the arthropods by oral ingestion. Suitable means for distributing the dusting powder to the locus of arthropod infestation include mechanical blowers, handshakers or livestock self treatment devices.

### Example 2H

An edible bait is prepared with the composition as follows:

| | |
|---|---|
| Active ingredient | 0.1 to 1.0% |
| Wheat flour | 80% |
| Molasses | 19.9 to 19% |

The ingredients are intimately mixed and formed as required into a bait form. This edible bait may be distributed at a locus, for example domestic or industrial premises, e.g. kitchens, hospitals or stores, or outdoor areas, infested by arthropods, for example ants, locusts, cockroaches or flies, to control the arthropods by oral ingestion.

### Example 21

A solution formulation is prepared with a composition as follows:

| | |
|---|---|
| Active ingredient | 15% |
| Dimethyl sulfoxide | 85% |

The active ingredient is dissolved in dimethyl sulfoxide with mixing and or heating as required. This solution may be applied percutaneously as a pour-on application to domestic animals infested by arthropods or, after sterilization by filtration through a polytetrafluoroethylene membrane (0.22 micrometer pore size), by parenteral injection, at a rate of application of from 1.2 to 12 ml of solution per 100 kg of animal body weight.

### Example 2J

A wettable powder is prepared with the composition as follows:

| | |
|---|---|
| Active ingredient | 50% |
| Ethylan BCP | 5% |
| Aerosil | 5% |
| Celite PF | 40% |

The Ethylan BCP is absorbed onto the Aerosil which is then mixed with the other ingredients and ground in a hammer-mill to give a wettable powder, which may be diluted with water to a concentration of from 0.0011% to 2% by weight of the active compound and applied to a locus of infestation by arthropods, for example, dipterous larvae or plant nematodes, by spraying, or to domestic animals infested by, or at risk of infection by arthropods, by spraying or dipping, or by oral administration in drinking water, to control the arthropods.

### Example 2K

A slow release bolus composition is formed from granules containing the following components in varying percentages (similar to those described for the previous compositions) depending upon need:

Active ingredient
Density agent
Slow-release agent
Binder

The intimately mixed ingredients are formed into granules which are compressed into a bolus with a specific gravity of 2 or more. This can be administered orally to ruminant domestic animals for retention within the reticulo-rumen to give a continual slow release of active compound over an extended period of time to control infestation of the ruminant domestic animals by arthropods.

### Example 2L

A slow release composition in the form of granules, pellets, brickettes or the like can be prepared with compositions as follows:

| | |
|---|---|
| Active ingredient | 0.5 to 25% |
| Polyvinyl chloride | 75 to 99.5% |
| Dioctyl phthalate | (plasticizer) |

The components are blended and then formed into suitable shapes by melt-extrusion or molding. These composition are useful, for example, for addition to standing water or for fabrication into collars or eartags for attachment to domestic animals to control pests by slow release.

### Example 2M

A water dispersible granule is prepared with the composition as follows:

| Active ingredient | 85%(max) |
|---|---|
| Polyvinylpyrrolidone | 5% |
| Attapulgite clay | 6% |
| Sodium lauryl sulfate | 2% |
| Glycerine | 2% |

The ingredients are mixed as a 45% slurry with water and wet milled to a particle size of 4 microns, then spray-dried to remove water.

Methods of Pesticidal Use

The following representative test procedures, using compounds of the invention, were conducted to determine the parasiticidal and pesticidal activity of compounds of the invention.

Method A:

Germinated field bean seeds (Vicia faba) with seed roots were transferred into brown glass bottles filled with tap water and then populated with about 100 black bean aphids (Aphis fabae). Plants and aphids were then dipped into an aqueous solution of the formulated preparation to be examined for 5 seconds. After they had drained, plants and animals were stored in a climatized chamber (16 hours of light/day, 25° C., 40-60% relative atmospheric humidity). After 3 and 6 days of storage, the effect of the preparation on the aphids was determined. At a concentration of 300 ppm (based on the content of active compound), the following Compounds of the Invention caused a mortality of 90-100% among the aphids:
A-86, A-91, A-101, A-196, B-86, B-87, B-91, B-201, B-246, B-286, B-455, B-456, B-464, B-529, B-536, E-129, E-130, E-135, E-137, E-138, E-143, E-185, E-325 and E-694.
   Method B:

Germinated field bean seeds (Vicia faba) with seed roots were transferred into brown glass bottles filled with tap water. Four milliliters of an aqueous solution of the formulated preparation to be examined were pipetted into the brown glass bottle. The field bean was then heavily populated with about 100 black bean aphids (Aphis fabae). Plants and aphids were then stored in a climatized chamber (16 hours of light/day, 25° C., 40-60% relative atmospheric humidity). After 3 and 6 days of storage, the root-systemic effect of the preparation on the aphids was determined. At a concentration of 30 ppm (based on the content of active compound), the following Compounds of the Invention caused a mortality of 90-100% among the aphids, by root-systemic action:
A-86, A-91, A-196, B-86, B-87, B-91, B-121, B-246, B-439, B-455, B-464, B-472, B-529, B-536, E-129, E-130, E-135, E-137, E-138, E-143, E-185, E-325 and E-694.

1. A compound of the formula (I): 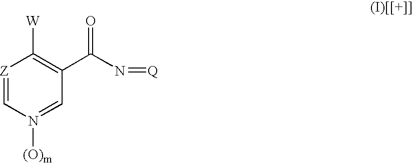
or a pesticidally acceptable salt thereof,
wherein:
W is (C₁-C₄)haloalkyl;
Z is CH or N;
=Q is a group of formula (A) or (B): 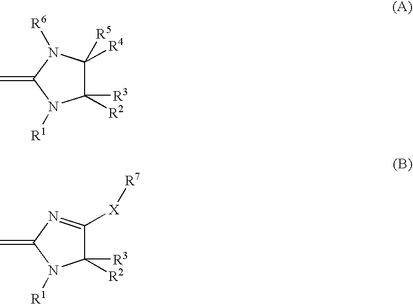
R¹ and R⁶ are each independently H, (C₁-C₈)alkyl, (C₃-C₆)alkenyl, (C₃-C₆)alkynyl, (C₁-C₆)alkoxy, (C₃-C₆)alkenyloxy, (C₃-C₆)alkynyloxy, (C₁-C₆)alkylamino, di-(C₁-C₆)alkylamino, NHCO(C₁-C₆)alkyl, NHSO₂(C₁-C₆)alkyl, CO(C₁-C₆)alkyl or SO₂(C₁-C₆)alkyl, wherein any available carbon on R¹ and R⁶ can be unsubstituted or substituted by one or more R⁸ groups; or
R¹ and R⁶ are (C₃-C₈)cycloalkyl or (C₃-C₈)cycloalkyl-(C₁-C₆)alkyl- which cycloalkyl radicals are unsubstituted or substituted by one or more (C₁-C₆)alkyl, (C₁-C₆)haloalkyl or R⁸ groups; or
R¹ and R⁶ are —(CR⁹R¹⁰)ₚR¹¹, —(CR⁹R¹⁰)ₚheterocyclyl, OH, SO₂R¹¹, NH₂, NHCOR¹¹, NH(C₃-C₈)cycloalkyl, NH(CR⁹R¹⁰)ₛR¹¹, O(CR⁹R¹⁰)ᵣR¹¹, —(CR⁹R¹⁰)CO₂CH₂R¹¹, O(CH₂)ᵣheterocyclyl, N═C[(C₁-C₆)alkyl]₂, COR^{11a} or CO-heterocyclyl; or
R¹ and R⁶ are (C₃-C₆)alkenyl substituted by R^{11a};
R², R³, R⁴ and R⁵ are each independently H, (C₁-C₈)alkyl, (C₂-C₆)alkenyl or (C₂-C₆)alkynyl, wherein any available carbon on R², R³, R⁴ or R⁵ can be unsubstituted or substituted by one or more R⁸ groups; or
R², R³, R⁴ and R⁵ are (C₃-C₈)cycloalkyl or (C₃-C₈)cycloalkyl-(C₁-C₆)alkyl- which cycloalkyl radicals are unsubstituted or substituted by one or more (C₁-C₆)alkyl, (C₁-C₆)haloalkyl or R⁸ groups; or
R², R³, R⁴ and R⁵ are (C₁-C₆)alkyl-SH, —(CR⁹R¹⁰)ₚR¹¹, —(CR⁹R¹⁰)ₚheterocyclyl or O(CH₂)ᵣR¹¹;
or R² and R³, or R⁴ and R⁵ together with the respective attached carbon atom form a carbonyl or thiocarbonyl group or a (C₃-C₈)cycloalkyl ring; or an imino group which is unsubstituted or substituted by (C₁-C₆)alkyl, CO(C₁-C₆)alkyl or R^{11a};
R⁷ is (C₃-C₆)alkenyl, (C₃-C₆)alkynyl, —(CR⁹R¹⁰)ₚR¹¹, —(CR⁹R¹⁰)ₚheterocyclyl, CO(C₁-C₆)alkyl or a (C₃-C₈)cycloalkyl ring; or (C₁-C₈)alkyl unsubstituted or substituted by one or more radicals selected from halogen and —OC(═O)—(C₁-C₄)alkyl;
R⁸ is halogen, (C₁-C₆)alkoxy, (C₁-C₆)haloalkoxy, S(O)ₙR¹², CN, CO₂(C₁-C₆)alkyl, CO₂H, NO₂, OH, amino, (C₁-C₆)alkylamino, di-(C₁-C₆)alkylamino, carbamoyl, (C₁-C₆)-alkylcarbamoyl, di-(C₁-C₆)-alkylcarbamoyl, CH[O(C₁-C₆)alkyl]₂, (C₃-C₆)alkenyloxy, (C₃-C₆)alkynyloxy or O(CH₂)ᵣR¹¹;
R⁹ and R¹⁰ are each independently H, (C₁-C₆)alkyl or (C₁-C₆)haloalkyl;
R¹¹ is aryl unsubstituted or substituted by one or more radicals selected from the group consisting of (C₁-C₆)alkyl, (C₁-C₆)haloalkyl, (C₂-C₆)alkenyl, (C₂-C₆)alkynyl, (C₃-C₈)cycloalkyl, —(CH₂)ᵤR^{11a}, heterocyclyl, halogen, (C₁-C₆)alkoxy, (C₁-C₆)haloalkoxy, S(O)ₙR¹², CN, CO₂(C₁-C₆)alkyl, NO₂, amino, (C₁-C₆)alkylamino, di-(C₁-C₆)alkylamino and CO(C₁-C₆)alkyl;
R^{11a} is aryl unsubstituted or substituted by one or more radicals selected from the group consisting of (C₁-C₆)alkyl, (C₁-C₆)haloalkyl, halogen, (C₁-C₆)alkoxy, (C₁-C₆)haloalkoxy, S(O)ₙR¹², CN, CO₂(C₁-C₆)alkyl, CO₂H, NO₂, OH, amino, (C₁-C₆)alkylamino and di-(C₁-C₆)alkylamino;
R¹² is (C₁-C₆)alkyl or (C₁-C₆)haloalkyl;
X is O, S, NR¹³ or NOR¹³;
R¹³ is H, (C₁-C₈)alkyl, (C₃-C₆)alkenyl, (C₃-C₆)alkynyl or (C₃-C₈)cycloalkyl wherein any available carbon on R¹³ can be unsubstituted or substituted by one or more R⁸ groups; or
R¹³ is (C₃-C₈)cycloalkyl-(C₁-C₆)alkyl- which cycloalkyl is unsubstituted or substituted by one or more (C₁-C₆)alkyl, (C₁-C₆)haloalkyl or R⁸ groups; or
R¹³ is —(CR⁹R¹⁰)ₚR¹¹ or —(CR⁹R¹⁰)ₚheterocyclyl;
m, s and u are each independently 0 or 1;
n is 0, 1 or 2;
p is 0, 1, 2 or 3;
r is 0 or an integer from 1 to 6; and each heterocyclyl in the above mentioned radicals is independently a heterocyclic radical having 3 to 7 ring atoms and 1 to 4 hetero atoms selected from the group consisting of N, O and S, and is unsubstituted or substituted by one or more radicals selected from the group consisting of (C₁-C₆)alkyl, (C₁-C₆)haloalkyl, (C₂-C₆)alkenyl, (C₂-C₆)alkynyl, —(CH₂)ᵤR^{11a}, halogen, (C₁-C₆)alkoxy, (C₁-C₆)haloalkoxy, S(O)ₙR¹², CN, CO₂(C₁-C₆)alkyl, NO₂, OH, amino, (C₁-C₆)alkylamino and di-(C₁-C₆)alkylamino.

2. A compound or a salt thereof as claimed in claim 1, wherein W is CF₃.

3. A compound or a salt thereof as claimed in claim 1 or 2, wherein Z is CH.

4. A compound or a salt thereof as claimed in claim 1, 2 or 3, wherein R¹ and R⁶ are each independently selected from the group consisting of H, (C₁-C₈)alkyl, (C₃-C₆)alkenyl, CO(C₁-C₆)alkyl, SO₂C₁-C₆)alkyl and —(CR⁹R¹⁰)ₚR¹¹.

5. A compound or a salt thereof as claimed in claim 1, wherein R², R³, R⁴ and R⁵ are each independently selected from the group consisting of H, (C₁-C₈)alkyl, (C₃-C₆)alkenyl, (C₃-C₆)alkynyl, —(CR⁹R¹⁰)ₚR¹¹, —(CR⁹R¹⁰)ₚheterocyclyl and O(CH₂)ᵣR¹¹; or R² and R³ together with the attached carbon atom form a carbonyl or thiocarbonyl group, or an imino group which is unsubstituted or substituted by (C₁-C₆)alkyl, CO(C₁-C₆)alkyl or R^{11a}; or R² and R³, or R⁴ and R⁵ together with the respective attached carbon atom form a (C₃-C₈)cycloalkyl ring.

6. A compound or a salt thereof as claimed in claim 1 wherein:
W is CF₃;
Z is CH;
R¹ and R⁶ are each independently selected from the group consisting of H, (C₁-C₈)alkyl, (C₃-C₆)alkenyl, CO(C₁-C₆)alkyl, SO₂(C₁-C₆)alkyl and —(CR⁹R¹⁰)ₚR¹¹;
R², R³, R⁴ and R⁵ are each independently selected from the group consisting of H, (C₁-C₈)alkyl, (C₃-C₆)alkenyl, (C₃-C₆)alkynyl, —(CR⁹R¹⁰)ₚR¹¹, —(CR⁹R¹⁰)ₚheterocyclyl and O(CH₂)ᵣR¹¹; or R² and R³ together with the attached carbon atom form a carbonyl or thiocarbonyl group, or an imino group which is unsubstituted or substituted by (C₁-C₆)alkyl, CO(C₁-C₆)alkyl or R^{11a}; or R² and R³, or R⁴ and R⁵ together with the respective attached carbon atom form a (C₃-C₈)cycloalkyl ring;
R⁷ is (C₁-C₈)alkyl, (C₃-C₆)alkenyl, (C₃-C₆)alkynyl, —(CR⁹R¹⁰)ₚR¹¹ or —(CR⁹R¹⁰)ₚheterocyclyl;
R⁸ is (C₁-C₄)alkoxy or OH;
R⁹ and R¹⁰ are each independently selected from the group consisting of H, (C₁-C₄)alkyl and (C₁-C₄)haloalkyl;
R¹¹ is phenyl unsubstituted or substituted by one or more radicals selected from the group consisting of (C₁-C₄)alkyl, (C₁-C₄)haloalkyl, (C₂-C₄)alkenyl, (C₂-C₄)alkynyl, (C₃-C₆)cycloalkyl, —(CH₂)ᵤR^{11a}, heterocyclyl, halogen, (C₁-C₄)alkoxy, (C₁-C₄)haloalkoxy, S(O)ₙR¹², CN, CO₂(C₁-C₄)alkyl, NO₂, amino, (C₁-C₄)alkylamino and di-(C₁-C₄)alkylamino;
R^{11a} is phenyl unsubstituted or substituted by one or more radicals selected from the group consisting of (C₁-C₄)alkyl, (C₁-C₄)haloalkyl, halogen, (C₁-C₄)alkoxy, (C₁-C₄)haloalkoxy, S(O)ₙR¹², CN, CO₂(C₁-C₄)alkyl, CO₂H, NO₂, OH, amino, (C₁-C₄)alkylamino and di-(C₁-C₄)alkylamino;
R¹² is (C₁-C₄)alkyl or (C₁-C₄)haloalkyl;
X is O or S;
m is 0; and
p, r, s and u are each independently 0 or 1;
wherein each heterocyclyl in the above mentioned radicals is independently a heterocyclic radical having 3 to 7 ring atoms and 1 to 4 hetero atoms selected from N, O and S.

7. A compound or a salt thereof as claimed in claim 1 wherein:
W is CF₃;
Z is CH;
=Q is a group of formula (A1): 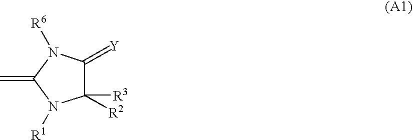
R¹ and R⁶ are each independently selected from the group consisting of H, (C₁-C₈)alkyl, (C₃-C₆)alkenyl, CO(C₁-C₆)alkyl, SO₂(C₁-C₆)alkyl and —(CR⁹R¹⁰)ₚR¹¹;
R² and R³ are each independently selected from the group consisting of H, (C₁-C₈)alkyl, (C₃-C₆)alkenyl, (C₃-C₆)alkynyl, —(CR⁹R¹⁰)ₚR¹¹, —(CR⁹R¹⁰)ₚheterocyclyl and O(CH₂)ᵣR¹¹; and
Y is O or S;
wherein said heterocyclyl is a heterocyclic radical having 3 to 7 ring atoms and 1 to 4 hetero atoms selected from N, O and S.

8. A process for the preparation of a compound of formula (I) or a salt thereof as defined in claim 1, which process comprises:
a) the cyclisation-rearrangement reaction of a compound of formula (II): 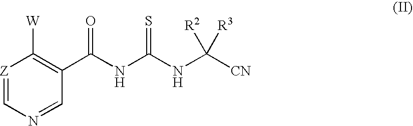
wherein W and Z are as defined in claim 1, R² and R³ are as defined in claim 1 excluding where together with the attached carbon atom they form a carbonyl, thiocarbonyl or imino group, by heating and/or reaction in the presence of a base, via an intermediate of formula (III): 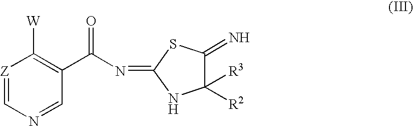
wherein W, Z, R² and R³ are as defined in claim 1, which rearranges to the compound of formula (I), where =Q is a formula (A), R² and R³ are as defined in claim 1 excluding where together with the attached carbon atom they form a carbonyl, thiocarbonyl or imino group, R⁴ and R⁵ together with the attached carbon atom form a thiocarbonyl group, R¹ and R⁶ are each a hydrogen atom and m is zero; or
b) reacting a compound of formula (IV): 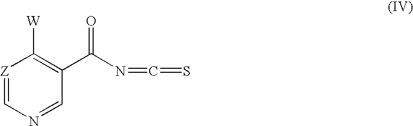
wherein W and Z are as defined in claim 1, with a compound of formula (V):
H₂NCR²(R³)CN (V)
wherein R² and R³ are as defined in claim 1 excluding where together with the attached carbon atom they form a carbonyl, thiocarbonyl or imino group, to give the corresponding compound of formula (II), followed by cyclisation and rearrangement as described in process a) above to give a compound of formula (D) where W and Z are as defined in claim 1, =Q is a formula (A), R² and R³ are as defined in claim 1 excluding where they form a carbonyl, thiocarbonyl or imino group, R⁴ and R⁵ together with the attached carbon atom form a thiocarbonyl group, R¹ and R⁶ are each a hydrogen atom and m is zero; or
c) reacting a compound of formula (VI): 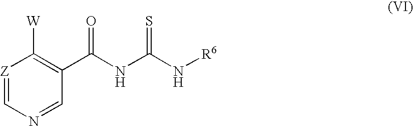
wherein W, Z and R⁶ are as defined in claim 1, with a compound of formula (VII):
H₂NCR²(R³)CO₂R⁷ (VII)
wherein R² and R³ are as defined in claim 1 excluding where together with the attached carbon atom they form a carbonyl, thiocarbonyl or imino group, and R⁷ is a leaving group, in the presence of a coupling agent to give an intermediate compound of formula (VIII): 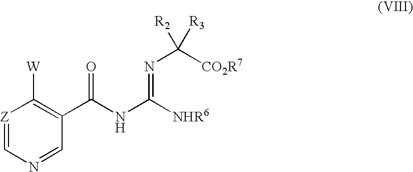
wherein the various symbols are as defined above, followed by cyclisation to give a compound of formula (I) where =Q is a formula (A), R¹ is a hydrogen atom, R² and R³ are as defined in claim 1 excluding where together with the attached carbon atom they form a carbonyl, thiocarbonyl or imino group, R⁴ and R⁵ together with the attached carbon atom form a carbonyl group, W, Z and R⁶ are as defined in claim 1 and m is zero; or
d) acylating the corresponding compound of formula (A¹) or (B¹): 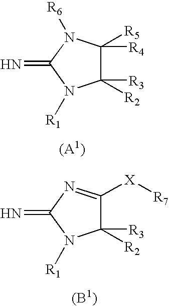
wherein the various symbols are as defined in claim 1, with a compound of formula (IX): 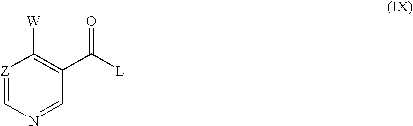
wherein W and Z are as defined in claim 1 and L is a leaving group to give a compound of formula (I) where =Q is a formula (A) or (B), m is zero and the other symbols are as defined in claim 1; or
e) reacting a compound of formula (I) which is of formula (X): 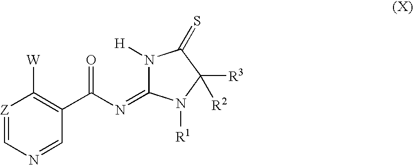
wherein W, Z, R¹, R² and R³ are as defined in claim 1, with a compound of formula (XI):
R⁷L (XI)
wherein R⁷ is as defined in claim 1 and L is a leaving group to give a compound of formula (I) where =Q is a formula (B), W, Z, R¹ and R⁷ are as defined in claim 1, X is S, m is zero, and R² and R³ are as defined in claim 1 excluding where together with the attached carbon atom they form a carbonyl or thiocarbonyl group, or an imino group which is unsubstituted or substituted by (C₁-C₆)alkyl, CO(C₁-C₆)alkyl or R^{11a}; or
f) cyclising a compound of formula (XII): 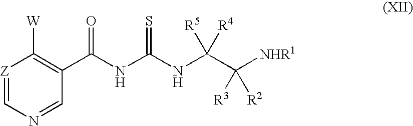
wherein W, Z, R¹, R², R³, R⁴ and R⁵ are as defined in claim 1, in the presence of a base to give a compound of formula (I) where =Q is a formula (A), W, Z, R¹, R², R³, R⁴ and R⁵ are as defined in claim 1, R⁶ is hydrogen and m is zero; or
g) oxidising and hydrolysing a compound of formula (I) wherein Q is a group of formula (B), X is S, and W, Z, R¹, R², R³ and R⁷ are as defined in claim 1, and m is zero to give a compound of formula (I) where =Q is a formula (A), W, Z, R¹, R² and R³ are as defined in claim 1, R⁴ and R⁵ together with the attached carbon atom form a carbonyl group, R⁶ is hydrogen, and m is zero; or
h) acylating the corresponding compound of formula (I) wherein R¹ is hydrogen, using a compound of formula (XIII):
R¹COL (XIII)
wherein L is a leaving group to give a compound of formula (I) where =Q is a formula (B), W, Z, R², R³ and R⁷ are as defined in claim 1, R¹ is CO(C₁-C₆)alkyl which is unsubstituted or substituted by one or more R⁸ groups, or is COR^{11a} or CO-heterocyclyl, and m is zero; or
i) acylating the corresponding compound of formula (I) wherein R¹ is hydrogen, using a compound of formula (XIII) as defined above to give a compound of formula (I) where =Q is a group of formula (A), W, Z, R², R³, R⁴, R⁵ and R⁶ are as defined in claim 1, R¹ is CO(C₁-C₆)alkyl which is unsubstituted or substituted by one or more R⁸ groups, or is COR^{11a} or CO-heterocyclyl, and m is zero; or
j) oxidising a corresponding compound of formula (I) in which m is 0 to give a compound of formula (I) where Q is as defined in claim 1, and m is 1; and
optionally, converting a resulting compound of formula (I) into a pesticidally acceptable salt thereof.

9. A pesticidal composition comprising a compound of formula (I) or a pesticidally acceptable salt thereof as defined in claim 1, and a pesticidally acceptable diluent or carrier and/or surface active agent.

10. (canceled)

11. The compound of claim 6, wherein R¹¹ is phenyl substituted unsubstituted or substituted by one or more radicals selected from the group consisting of (C₁-C₄)alkyl, halogen, (C₁-C₄)alkoxy, NO₂ and amino.
